SEQUENCE LISTING (87 sequences)

SEQ ID NO: 1 - DNA, 20 bp, Unknown
  Description of Unknown target sequence
ctaccgaggctccagcttaa

SEQ ID NO: 2 - DNA, 20 bp, Unknown
  Description of Unknown target sequence
ggcaggaggaacggctaccg

SEQ ID NO: 3 - DNA, 20 bp, Unknown
  Description of Unknown target sequence
ggggaggagggcccgttggg

SEQ ID NO: 4 - DNA, 20 bp, Unknown
  Description of Unknown target sequence
ccaccgaggctccagcttaa

SEQ ID NO: 5 - DNA, 20 bp, Unknown
  Description of Unknown target sequence
ggcaagaagcatggccaccg

SEQ ID NO: 6 - DNA, 3249 bp, Artificial Sequence
  Description of Artificial Sequence Synthetic polynucleotide
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaatggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 7 - DNA, 2824 bp, Artificial Sequence
  Description of Artificial Sequence Synthetic polynucleotide
atgcagagggtgaacatgatcatggctgagagccctggcctgatcaccatctgcctgctgggctacctgctgtctgctgaatgtacaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattgggaaacaaaagcacaaacagtggccttatttacacaaaaagtctgattttaagatatgtgacaattcaaggtttcagaagtatgtaaggaggtgtgtctctaattttttaaattatatatcttcaatttaaagttttagttaaaacataaagattaacctttcattagcaagctgttagttatcaccaaagcttttcatggattaggaaaaaatcattttgtctctatctcaaacatcttggagttgatatttggggaaacacaatactcagttgagttccctaggggagaaaagcaagcttaagaattgacacaaagagtaggaagttagctattgcaacatatatcactttgttttttcacaactacagtgactttatttatttcccagaggaaggcatacagggaagaaattatcccatttggacaaacagcatgttctcacagtaagcacttatcacacttacttgtcaactttctagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgctgagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 8 - DNA, 2460 bp, Unknown
  Description of Unknown LDLR sequence
atggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgctcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtga

SEQ ID NO: 9 - DNA, 20 bp, Artificial Sequence
  Description of Artificial Sequence Synthetic primer
cccgaaaggaaagggtggcg

SEQ ID NO: 10 - DNA, 20 bp, Artificial Sequence
  Description of Artificial Sequence Synthetic primer
tggcacctgcacttgcactg

SEQ ID NO: 11 - DNA, 20 bp, Artificial Sequence
  Description of Artificial Sequence Synthetic primer
tccggggtgcacgagccgac

SEQ ID NO: 12 - DNA, 20 bp, Artificial Sequence
  Description of Artificial Sequence Synthetic primer
gcggtggctccactttccct

SEQ ID NO: 13 - DNA, 20 bp, Artificial Sequence
  Description of Artificial Sequence Synthetic primer
ggggtgcgggctgactttct

SEQ ID NO: 14 - DNA, 23 bp, Artificial Sequence
  Description of Artificial Sequence Synthetic primer
ctgagacaggtaaacacctccat

SEQ ID NO: 15 - DNA, 18 bp, Artificial Sequence
  Description of Artificial Sequence Synthetic primer
tggagacgtcctggcccc

SEQ ID NO: 16 - DNA, 19 bp, Artificial Sequence
  Description of Artificial Sequence Synthetic primer
cctggcacgtttgctgagg

SEQ ID NO: 17 - DNA, 20 bp, Artificial Sequence
  Description of Artificial Sequence Synthetic primer
tagttgccagccatctgttg

SEQ ID NO: 18 - DNA, 20 bp, Artificial Sequence
  Description of Artificial Sequence Synthetic primer
ggggacagcctattttgcta

SEQ ID NO: 19 - DNA, 20 bp, Artificial Sequence
  Description of Artificial Sequence Synthetic probe
aaatgaggaaattgcatcgc

SEQ ID NO: 20 - DNA, 20 bp, Artificial Sequence
  Description of Artificial Sequence Synthetic primer
tagtgggaacgatgggggat

SEQ ID NO: 21 - DNA, 19 bp, Artificial Sequence
  Description of Artificial Sequence Synthetic primer
agtccaagctgagcaaaga

SEQ ID NO: 22 - DNA, 26 bp, Artificial Sequence
  Description of Artificial Sequence Synthetic probe
cgagaagcgcgatcacatggtcctgc

SEQ ID NO: 23 - DNA, 20 bp, Artificial Sequence
  Description of Artificial Sequence Synthetic primer
gctgcctatcagaaagtggt

SEQ ID NO: 24 - DNA, 24 bp, Artificial Sequence
  Description of Artificial Sequence Synthetic probe
ctggtgtggctaatgccctggccc

SEQ ID NO: 25 - DNA, 19 bp, Artificial Sequence
  Description of Artificial Sequence Synthetic primer
atcacaaacgcaggcagag

SEQ ID NO: 26 - DNA, 19 bp, Artificial Sequence
  Description of Artificial Sequence Synthetic primer
gctgtatgaatccaggtcc

SEQ ID NO: 27 - DNA, 19 bp, Artificial Sequence
  Description of Artificial Sequence Synthetic primer
cctcctggctgagaaaaag

SEQ ID NO: 28 - DNA, 26 bp, Artificial Sequence
  Description of Artificial Sequence Synthetic probe
tgtttcctccaggataaggcagctgt

SEQ ID NO: 29 - DNA, 18 bp, Artificial Sequence
  Description of Artificial Sequence Synthetic primer
gagaacttcaggctcctg

SEQ ID NO: 30 - DNA, 20 bp, Artificial Sequence
  Description of Artificial Sequence Synthetic primer
cgggggtacgggtgcaggaa

SEQ ID NO: 31 - DNA, 20 bp, Artificial Sequence
  Description of Artificial Sequence Synthetic probe
tggccatgcttcttgcccct

SEQ ID NO: 32 - DNA, 21 bp, Artificial Sequence
  Description of Artificial Sequence Synthetic primer
gacctgcacgcgcacaagctt

SEQ ID NO: 33 - DNA, 22 bp, Artificial Sequence
  Description of Artificial Sequence Synthetic primer
gctcacagaagccaggaacttg

SEQ ID NO: 34 - DNA, 21 bp, Artificial Sequence
  Description of Artificial Sequence Synthetic probe
caacttcaagctcctaagcca

SEQ ID NO: 35 - DNA, 23 bp, Artificial Sequence
  Description of Artificial Sequence Synthetic primer
atatgcagccactcctagagctc

SEQ ID NO: 36 - DNA, 23 bp, Artificial Sequence
  Description of Artificial Sequence Synthetic primer
ctggttcagagaaatgatgggca

SEQ ID NO: 37 - DNA, 20 bp, Artificial Sequence
  Description of Artificial Sequence Synthetic probe
aggaaaccggagaaagggta

SEQ ID NO: 38 - DNA, 4599 bp, Artificial Sequence
  Description of Artificial Sequence Synthetic polynucleotide
tttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctctagataccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcaggatccatgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctcagacctgcacttagattgccttcccttttgttggtccacgtttgcgctaggcccgagaaaccgccaggacaagtaacacggcttcgggcgctgccacttactcaggggcagctggtgctggtttggtcagacgagcatgtcggaagcaaatgcctttggacctacgagatacaattttcacaggatggtaaggcttacactccggtctcaagaaagcccagtacctttaacctttttgtgttcagtccagatactggagcagtaagcggttcatatagagtcagagcgctggattactgggccaggcccggacctttctcagatccggtcccctacctggaagttcccgtgccgcggggtcctccatcaccaggcaacccaggaagcggagctactaacttcagcctgctgaagcaggctggagacgtggaggagaaccctggacctggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcgggtgtggtgaccacagtgatgggcagctcccagcccgtggtgacccgaggcaccaccgacaacctcatccctgtctattgctccatcctggctgctgtggttgtgggtcttgtggcctacatagccttcaagaggtaataactcgagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctactagttgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaa

SEQ ID NO: 39 - DNA, 3255 bp, Artificial Sequence
  Description of Artificial Sequence Synthetic polynucleotide
gtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcctgaggagaagtctgccgttactgcccattaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggtaccgagctcttcgaaggatccatcgccaccatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagccggcagcggcgagggccgcggcagcctgctgacctgcggcgacgtggaggagaaccccggccccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacgtttcgcgcctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgct

SEQ ID NO: 40 - DNA, 23 bp, Artificial Sequence
  Description of Artificial Sequence Synthetic oligonucleotide
  modified_base (21)..(21): a, c, t, g, unknown or other
ctaccgaggctccagcttaangg

SEQ ID NO: 41 - DNA, 23 bp, Artificial Sequence
  Description of Artificial Sequence Synthetic oligonucleotide
  modified_base (21)..(21): a, c, t, g, unknown or other
ggcaggaggaacggctaccgngg

SEQ ID NO: 42 - DNA, 23 bp, Artificial Sequence
  Description of Artificial Sequence Synthetic oligonucleotide
  modified_base (21)..(21): a, c, t, g, unknown or other
ggggaggagggcccgttgggngg

SEQ ID NO: 43 - DNA, 23 bp, Artificial Sequence
  Description of Artificial Sequence Synthetic oligonucleotide
  modified_base (21)..(21): a, c, t, g, unknown or other
ccaccgaggctccagcttaangg

SEQ ID NO: 44 - DNA, 23 bp, Artificial Sequence
  Description of Artificial Sequence Synthetic oligonucleotide
  modified_base (21)..(21): a, c, t, g, unknown or other
ggcaagaagcatggccaccgngg

SEQ ID NO: 45 - DNA, 23 bp, Unknown
  Description of Unknown target sequence
  modified_base (21)..(21): a, c, t, g, unknown or other
ggcaagaagcatggccaccgngg

SEQ ID NO: 46 - DNA, 23 bp, Unknown
  Description of Unknown off-target sequence
  modified_base (21)..(21): a, c, t, g, unknown or other
gcaagaagcaagggccaccgngg

SEQ ID NO: 47 - DNA, 22 bp, Unknown
  Description of Unknown off-target sequence
  modified_base (20)..(20): a, c, t, g, unknown or other
ggcaggagcatggcctccgngg

SEQ ID NO: 48 - DNA, 22 bp, Unknown
  Description of Unknown off-target sequence
  modified_base (20)..(20): a, c, t, g, unknown or other
ggcaagagcctggcccccgngg

SEQ ID NO: 49 - DNA, 22 bp, Unknown
  Description of Unknown off-target sequence
  modified_base (20)..(20): a, c, t, g, unknown or other
gacacgaagcatggccacgngg

SEQ ID NO: 50 - DNA, 23 bp, Unknown
  Description of Unknown off-target sequence
  modified_base (21)..(21): a, c, t, g, unknown or other
ggccagaagcctggccaacgngg

SEQ ID NO: 51 - DNA, 22 bp, Unknown
  Description of Unknown off-target sequence
  modified_base (20)..(20): a, c, t, g, unknown or other
ggccagaatcatggccacgngg

SEQ ID NO: 52 - DNA, 23 bp, Unknown
  Description of Unknown off-target sequence
  modified_base (21)..(21): a, c, t, g, unknown or other
acaagcagcatggccaaccgngg

SEQ ID NO: 53 - DNA, 23 bp, Unknown
  Description of Unknown off-target sequence
  modified_base (21)..(21): a, c, t, g, unknown or other
gcatgaagcatggatcaccgngg

SEQ ID NO: 54 - DNA, 22 bp, Unknown
  Description of Unknown off-target sequence
  modified_base (20)..(20): a, c, t, g, unknown or other
ggcaagaagcatggacccgngg

SEQ ID NO: 55 - DNA, 23 bp, Unknown
  Description of Unknown off-target sequence
  modified_base (21)..(21): a, c, t, g, unknown or other
gcaatgcagcatggccactgngg

SEQ ID NO: 56 - DNA, 23 bp, Unknown
  Description of Unknown off-target sequence
  modified_base (21)..(21): a, c, t, g, unknown or other
ggtgagaagcatggccaccangg

SEQ ID NO: 57 - DNA, 22 bp, Unknown
  Description of Unknown off-target sequence
  modified_base (20)..(20): a, c, t, g, unknown or other
ggcagaagcctggccacagngg

SEQ ID NO: 58 - DNA, 22 bp, Unknown
  Description of Unknown off-target sequence
  modified_base (20)..(20): a, c, t, g, unknown or other
ggccagaagctggccacggngg

SEQ ID NO: 59 - DNA, 22 bp, Unknown
  Description of Unknown off-target sequence
  modified_base (20)..(20): a, c, t, g, unknown or other
ggcagaagcaaggccacagngg

SEQ ID NO: 60 - DNA, 22 bp, Unknown
  Description of Unknown off-target sequence
  modified_base (20)..(20): a, c, t, g, unknown or other
tgcaaaagcatggccaccangg

SEQ ID NO: 61 - DNA, 22 bp, Unknown
  Description of Unknown off-target sequence
  modified_base (20)..(20): a, c, t, g, unknown or other
ggaagcagcatggccacccngg

SEQ ID NO: 62 - DNA, 22 bp, Unknown
  Description of Unknown off-target sequence
  modified_base (20)..(20): a, c, t, g, unknown or other
gtcaagaagcatggctacgngg

SEQ ID NO: 63 - DNA, 22 bp, Unknown
  Description of Unknown off-target sequence
  modified_base (20)..(20): a, c, t, g, unknown or other
ggcaaaaccatggccacccngg

SEQ ID NO: 64 - DNA, 22 bp, Unknown
  Description of Unknown off-target sequence
  modified_base (20)..(20): a, c, t, g, unknown or other
ggtaagaagctggccaccangg

SEQ ID NO: 65 - DNA, 22 bp, Unknown
  Description of Unknown off-target sequence
  modified_base (20)..(20): a, c, t, g, unknown or other
ggcaagatgcaggccaccangg

SEQ ID NO: 66 - DNA, 23 bp, Unknown
  Description of Unknown off-target sequence
  modified_base (21)..(21): a, c, t, g, unknown or other
ggcaagaagcctgggcaccangg

SEQ ID NO: 67 - DNA, 22 bp, Unknown
  Description of Unknown off-target sequence
  modified_base (20)..(20): a, c, t, g, unknown or other
gggcagaagcatggccccgngg

SEQ ID NO: 68 - DNA, 22 bp, Unknown
  Description of Unknown off-target sequence
  modified_base (20)..(20): a, c, t, g, unknown or other
ggcaagaagctggcaacctngg

SEQ ID NO: 69 - DNA, 22 bp, Unknown
  Description of Unknown off-target sequence
  modified_base (20)..(20): a, c, t, g, unknown or other
ggcaggaagcatggccccangg

SEQ ID NO: 70 - DNA, 23 bp, Unknown
  Description of Unknown off-target sequence
  modified_base (21)..(21): a, c, t, g, unknown or other
gcaagaaggcatggccagggngg

SEQ ID NO: 71 - DNA, 23 bp, Unknown
  Description of Unknown off-target sequence
  modified_base (21)..(21): a, c, t, g, unknown or other
ggcaagaagcatgtccagtgngg

SEQ ID NO: 72 - DNA, 22 bp, Unknown
  Description of Unknown off-target sequence
  modified_base (20)..(20): a, c, t, g, unknown or other
ggcaagaagcatggctctgngg

SEQ ID NO: 73 - DNA, 22 bp, Unknown
  Description of Unknown off-target sequence
  modified_base (20)..(20): a, c, t, g, unknown or other
ggcaagaagcatgccaaagngg

SEQ ID NO: 74 - DNA, 22 bp, Unknown
  Description of Unknown off-target sequence
  modified_base (20)..(20): a, c, t, g, unknown or other
ggcaagaagatggccatctngg

SEQ ID NO: 75 - DNA, 22 bp, Unknown
  Description of Unknown off-target sequence
  modified_base (20)..(20): a, c, t, g, unknown or other
ggcaagaagcatggctccangg

SEQ ID NO: 76 - DNA, 22 bp, Unknown
  Description of Unknown off-target sequence
  modified_base (20)..(20): a, c, t, g, unknown or other
ggcaagaagcatggcaaagngg

SEQ ID NO: 77 - DNA, 23 bp, Unknown
  Description of Unknown off-target sequence
  modified_base (21)..(21): a, c, t, g, unknown or other
ggcaagcagcttggccaccgngt

SEQ ID NO: 78 - DNA, 22 bp, Unknown
  Description of Unknown off-target sequence
  modified_base (20)..(20): a, c, t, g, unknown or other
ggccagaagctggccaccgncg

SEQ ID NO: 79 - DNA, 22 bp, Unknown
  Description of Unknown off-target sequence
  modified_base (20)..(20): a, c, t, g, unknown or other
ggcaggaagcatgccaccgngc

SEQ ID NO: 80 - DNA, 22 bp, Unknown
  Description of Unknown off-target sequence
  modified_base (20)..(20): a, c, t, g, unknown or other
ggcaaaagcatggacaccgnag

SEQ ID NO: 81 - DNA, 23 bp, Unknown
  Description of Unknown off-target sequence
  modified_base (21)..(21): a, c, t, g, unknown or other
gcaagaagcaatggcaaccgnag

SEQ ID NO: 82 - DNA, 22 bp, Unknown
  Description of Unknown off-target sequence
  modified_base (20)..(20): a, c, t, g, unknown or other
ggcaagaagcagggccccgnga

SEQ ID NO: 83 - DNA, 22 bp, Unknown
  Description of Unknown off-target sequence
  modified_base (20)..(20): a, c, t, g, unknown or other
ggcaggaagcatggccacanag

SEQ ID NO: 84 - DNA, 23 bp, Unknown
  Description of Unknown off-target sequence
  modified_base (21)..(21): a, c, t, g, unknown or other
ggcaagaagcattgccacagntg

SEQ ID NO: 85 - DNA, 23 bp, Unknown
  Description of Unknown off-target sequence
  modified_base (21)..(21): a, c, t, g, unknown or other
gcaagcaagcatggccacctnag

SEQ ID NO: 86 - DNA, 130 bp, Unknown
  Description of Unknown target sequence
caaataccgttaagctggagcctcggtagccgttcctcctgcccgctgggcctcccaacgggccctcctcccctccttgcaccggcccttcctggtctttgaataaagtctgagtgggcagcagcctgtg

SEQ ID NO: 87 - DNA, 130 bp, Unknown
  Description of Unknown target sequence
ctggacaagttcctggcttctgtgagcaccgtgctgacctccaaataccgttaagctggagcctcggtggccatgcttcttgccccttgggcctccccccagcccctcctccccttcctgcacccgtacc

(19) United States
(12) Patent Application Publication
(10) Pub. No.: US 2022/0356450 A1
(43) Pub. Date: Nov. 10, 2022

(54) TARGETED INTEGRATION AT ALPHA-GLOBIN LOCUS IN HUMAN HEMATOPOIETIC STEM AND PROGENITOR CELLS

(71) Applicant: THE BOARD OF TRUSTEES OF THE LELAND STANFORD JUNIOR UNIVERSITY, Stanford, CA (US)

(72) Inventors: MATTHEW H PORTEUS, Stanford, CA (US); MICHAEL KYLE CROMER, Stanford, CA (US); DANIEL P. DEVER, Stanford, CA (US); JOAB CAMARENA, Stanford, CA (US)

(73) Assignee: THE BOARD OF TRUSTEES OF THE LELAND STANFORD JUNIOR UNIVERSITY, Stanford, CA (US)

(21) Appl. No.: 17/740,102

(22) Filed: May 9, 2022

Related U.S. Application Data
(63) Continuation of application No. PCT/US2020/060586, filed on Nov. 13, 2020.
(60) Provisional application No. 62/936,248, filed on Nov. 15, 2019.

(51) Int. Cl. C12N 5/0789; C12N 15/861; C12N 15/11

(52) U.S. Cl. CPC C12N 5/0647; C12N 15/861; C12N 15/11

(57) ABSTRACT

The present disclosure provides methods and compositions for genetically modifying hematopoietic stem and progenitor cells (HSPCs), in particular by replacing the HBA1 or HBA2 locus in the HSPCs with a transgene encoding a therapeutic protein.

### CROSS REFERENCE TO RELATED APPLICATIONS

The present application is a continuation of International Appl. No. PCT/US2020/060586, filed Nov. 13, 2020, which claims the benefit of priority to U.S. Provisional Pat. Appl. No. 62/936,248, filed on Nov. 15, 2019, the contents of which are incorporated herein by reference in their entireties.

### STATEMENT AS TO RIGHTS TO INVENTIONS MADE UNDER FEDERALLY SPONSORED RESEARCH AND DEVELOPMENT

This invention was made with Government support under Grant No. HL135607 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 7, 2021, is named 1211943_SL.txt and is 44,500 bytes in size.

### BACKGROUND

β-thalassemia is one of the most common genetic blood disorders in the world, with a global incidence of 1 in 100,000 (1). Patients with this disease suffer from severe anemia and, even with intensive medical care, experience a median life expectancy of 30 years of age (2-4). The most severe form of the disease-β-thalassemia major—is caused by homozygous (or compound heterozygous) loss-of-function mutations throughout the β-globin (HBB) gene. This results in loss of HBB protein, causing profound anemia and reducing the ability of red blood cells (RBCs) to deliver oxygen throughout the body. The accumulation of unpaired α-globin (from the HBA1 and HBA2 genes) leads to dramatic erythrotoxicity, contributing to the anemia seen in patients. In fact, disease severity is known to be directly correlated with the degree of imbalance between β-globin and α-globin chains (5). The current standard of care for β-thalassemia involves frequent blood transfusions combined with iron chelation therapy, making this one of the costliest genetic diseases in young adults (6). Currently, the only curative strategy for this disease is allogeneic hematopoietic stem cell transplantation (HSCT) from an immunologically-matched donor. However, in the majority of cases no matched donor is available for allogeneic HSCT, and, even if one is identified, transplants from these donors carry a risk of immune rejection and graft-versus-host disease (7).

A potentially ideal treatment would involve isolation of patient-derived hematopoietic stem and progenitor cells (HSPCs), introduction of HBB to restore HBB protein levels, followed by autologous HSCT of the patient's own corrected HSPCs, which would carry no risk of immune rejection. Employing this logic, several gene therapies have been developed as a potentially curative measure for β-thalassemia, primarily through the delivery of an HBB transgene using lentiviral vectors (8, 9). While these approaches have been shown to restore HBB to therapeutic levels in human clinical trials for β-thalassemia (10), delivery with lenti- and retroviral vectors results in semi-random genomic integration, which is capable of deactivating tumor suppressor genes or activating oncogenes. In fact, semi-random integration in HSPCs has been shown to lead to clonal expansion, myelodysplasia, and acute myeloid leukemia (11-14), one instance of which proved fatal (15). Moreover, the lentiviral gene therapy approach, while reaching registration status for milder forms of transfusion-dependent β-thalassemia, has not resulted in transfusion-independence for severe forms of the disease.

Because of these remaining safety and efficacy issues, alternative strategies have been developed that employ genome editing (including zinc finger nucleases and the CRISPR/Cas9 system) to initiate site-specific DNA double-strand breaks (DSBs) in order to inactivate a repressor of fetal hemoglobin, the upregulation of which could compensate for the lack of HBB (16). However, there is some concern that the resulting upregulation of fetal hemoglobin may not be sufficient to rescue the β-globin:α-globin imbalance and that the upregulation may not persist in adult patients, in whom fetal hemoglobin is naturally silenced (17, 18). Moreover, this approach does not address the genetic cause of β-thalassemia— inactivation of HBB—and may not sufficiently rescue the disease phenotype in vivo. Furthermore, all of these therapies act to compensate only for the lack of HBB, and do not diminish levels of α-globin.

There is thus a need for new, safe and effective approaches for introducing HBB or other therapeutic transgenes into autologous HSPCs and red blood cells in vivo or ex vivo. The present disclosure satisfies this need and provides other advantages as well.

### BRIEF SUMMARY

In one aspect, the present disclosure provides a method of genetically modifying a hematopoietic stem and progenitor cell (HSPC) from a subject, the method comprising introducing into the HSPC a guide RNA comprising a sequence that hybridizes to a HBA1 gene sequence or a HBA2 gene sequence, an RNA-guided nuclease, and a donor template comprising a transgene encoding a protein, wherein the RNA-guided nuclease cleaves the HBA1 gene sequence or the HBA2 gene sequence, but not both, in the cell; wherein the transgene is integrated into the cleaved HBA1 gene sequence or HBA2 gene sequence; thereby generating a genetically modified HSPC, wherein the integrated transgene results in expression of the protein in the genetically modified HSPC.

In another aspect, the present disclosure provides a method of genetically modifying a hematopoietic stem and progenitor cell (HSPC) from a subject, the method comprising introducing into the HSPC a guide RNA comprising a sequence that hybridizes to a HBA1 gene sequence or a HBA2 gene sequence, an RNA-guided nuclease, and a donor template comprising a transgene encoding a protein, wherein the RNA-guided nuclease cleaves the HBA1 gene sequence or the HBA2 gene sequence, but not both, in the cell; wherein the transgene is integrated into the cleaved HBA1 gene sequence or HBA2 gene sequence; thereby generating a genetically modified HSPC, wherein the introduction results in reduced translocation events in a genome of the HSPC as compared to introduction of the RNA-guided nuclease, the donor template, and a guide RNA that hybridizes to both a HBA1 gene sequence and a HBA2 gene sequence.

In another aspect, the present disclosure provides a method of genetically modifying a hematopoietic stem and progenitor cell (HSPC) from a subject, the method comprising introducing into the HSPC a guide RNA comprising a sequence that hybridizes to a HBA1 gene sequence or a HBA2 gene sequence, an RNA-guided nuclease, and a donor template comprising a transgene encoding a protein, wherein the RNA-guided nuclease cleaves the HBA1 gene sequence or the HBA2 gene sequence, but not both, in the cell; wherein the transgene is integrated into the cleaved HBA1 gene sequence or HBA2 gene sequence; thereby generating a genetically modified HSPC, wherein the introduction results in reduced off-target integration of the donor template in a genome of the HSPC as compared to introduction of the RNA-guided nuclease, the donor template, and a guide RNA that hybridizes to both a HBA1 gene sequence and a HBA2 gene sequence.

In some embodiments of any of the herein-disclosed methods, the method further comprises isolating the HSPC from the subject prior to the introducing of the guide RNA, the RNA-guided nuclease, and the donor template. In some embodiments, the HBA1 gene sequence or the HBA2 gene sequence comprises a 3′ UTR region. In some embodiments, the RNA-guided nuclease cleaves the HBA1 gene sequence but not the HBA2 gene sequence. In some embodiments, the HBA1 gene sequence comprises a sequence of SEQ ID NO:5. In some embodiments, the transgene is integrated into the HBA1 gene sequence. In some embodiments, the RNA-guided nuclease cleaves the HBA2 gene sequence but not the HBA1 gene sequence. In some embodiments, the HBA2 gene sequence comprises a sequence of SEQ ID NO:2. In some embodiments, the transgene is integrated into the HBA2 gene sequence.

In some embodiments of any of the herein-disclosed methods, the HSPC comprises a HBB gene that comprises a mutation as compared to a wild type HBB gene. In some embodiments, the mutation is causative of a disease. In some embodiments, the disease is beta-thalassemia. In some embodiments, the transgene is selected from the group consisting of HBB, PDGFB, IDUA, FIX (e.g., the Padua variant), LDLR, and PAH. In some embodiments, the transgene is HBB. In some embodiments, the HBB is expressed in the HSPC and increases a level of adult hemoglobin tetramers in the HSPC as compared to prior to introduction of the guide RNA, the RNA-guided nuclease, and the donor template. In some embodiments, the transgene is HBB, wherein the guide RNA hybridizes to a sequence of SEQ ID NO:5, and wherein the HBB is integrated at the site of the HBA1 gene sequence.

In some embodiments, the subject has β-thalassemia, and the genetically modified HSPC expressing the HBB transgene is reintroduced into the subject. In some embodiments, the expression of the integrated transgene is driven by an endogenous HBA1 or HBA2 promoter. In some embodiments, the integrated transgene replaces the HBA1 or HBA2 coding sequence in a genome of the HSPC. In some embodiments, the integrated transgene replaces the HBA1 or HBA2 open reading frame (ORF) in a genome of the HSPC. In some embodiments, the protein is a secreted protein. In some embodiments, the protein is a therapeutic protein.

In some embodiments, the guide RNA comprises one or more 2′-O-methyl-3′-phosphorothioate (MS) modifications. In some such embodiments, the one or more 2′-O-methyl-3′-phosphorothioate (MS) modifications are present at the three terminal nucleotides of the 5′ and 3′ ends of the guide RNA. In some embodiments, the RNA-guided nuclease is Cas9. In some embodiments, the guide RNA and the RNA-guided nuclease are introduced into the HSPC as a ribonucleoprotein (RNP) complex by electroporation. In some embodiments, the donor template is introduced into the HSPC using a recombinant adeno-associated virus (rAAV) vector. In some such embodiments, the rAAV vector is a AAV6 vector.

In some embodiments, the introducing is performed ex vivo. In some embodiments, the method further comprises introducing the genetically modified HSPC into the subject. In some embodiments, the method further comprises inducing the genetically modified HSPC to differentiate in vitro or ex vivo into a red blood cell (RBC). In some embodiments, the subject is a human.

In another aspect, the present disclosure provides a guide RNA comprising a sequence that hybridizes to a HBA1 gene sequence or a HBA2 gene sequence, but not both. In some embodiments, the guide RNA hybridizes to a 3′ UTR of the HBA1 gene sequence or the HBA2 gene sequence. In some embodiments, the guide RNA hybridizes to the HBA1 gene sequence. In some embodiments, the HBA1 gene sequence comprises the sequence of SEQ ID NO: 5. In some embodiments, the guide RNA hybridizes to the HBA2 gene sequence. In some embodiments, the HBA2 gene sequence comprises the sequence of SEQ ID NO: 2. In some embodiments, the guide RNA comprises one or more 2′-O-methyl-3′-phosphorothioate (MS) modifications. In some such embodiments, the one or more 2′-O-methyl-3′-phosphorothioate (MS) modifications are present at the three terminal nucleotides of the 5′ and 3′ ends of the guide RNA.

In another aspect, the present disclosure provides an HSPC comprising any of the herein-disclosed guide RNAs.

In another aspect, the present disclosure provides a genetically modified HSPC comprising a transgene integrated in a HBA1 or HBA2 gene sequence, but not both. In some embodiments, the genetically modified HSPC is generated using any of the herein-disclosed methods. In some embodiments, the transgene is selected from the group consisting of HBB, PDGFB, IDUA, FIX (e.g., the Padua variant), LDLR, and PAH. In some embodiments, the transgene is HBB. In some embodiments, the HBB is integrated at the HBA1 gene sequence. In some embodiments, the HBB transgene has replaced the endogenous HBA1 coding sequence in a genome of the genetically modified HSPC. In some embodiments, the HBB transgene has replaced the endogenous HBA1 open reading frame in a genome of the genetically modified HSPC.

In another aspect, the present disclosure provides a red blood cell produced by inducing the differentiation in vitro or ex vivo of any of the herein-described genetically modified HSPCs.

In another aspect, the present disclosure provides a method for treating beta-thalassemia in a subject in need thereof, the method comprising administering any of the herein-disclosed genetically modified HSPCs to the subject, wherein the genetically modified HSPC engrafts in the subject and results in increased level of adult hemoglobin tetramers in the subject as compared to prior to the administration, thereby treating beta-thalassemia in the subject.

In some embodiments of the method, the genetically modified HSPC is derived from the subject.

In another aspect, the present disclosure provides a method of modifying a cell, the method comprising introducing into the cell a programmable nuclease that cleaves a target locus in a target gene in the cell; and a nucleic acid comprising a donor template comprising a transgene, wherein the transgene is integrated into the target locus, and wherein the transgene replaces a whole or a part of an open reading frame (ORF) of a protein encoded by the target gene.

In some embodiments, the transgene replaces a region of the target gene selected from the group consisting of: a 5′ UTR, one or more exons, one or more introns, a 3′ UTR, and any combination thereof. In some embodiments, the transgene replaces introns and exons of the target gene. In some embodiments, the cell is a primary cell. In some embodiments, the cell is a hematopoietic stem and progenitor cell (HSPC). In some embodiments, the transgene encodes a therapeutic protein. In some embodiments, the transgene is selected from the group consisting of HBB, PDGFB, IDUA, FIX (e.g., the Padua variant), LDLR, and PAH. In some embodiments, the transgene is HBB. In some embodiments, the target gene comprises a mutation associated with a disease. In some embodiments, the target gene comprises two or more mutations associated with a disease. In some embodiments, the target gene encodes a protein associated with the disease and wherein the transgene encodes a wild type of the protein. In some embodiments, the target gene is a safe harbor gene. In some embodiments, the target gene is an HBA1 gene. In some embodiments, the target gene is an HBA2 gene.

In some embodiments of any of the herein-disclosed methods, the transgene is flanked by a first homology arm and a second homology arm, wherein the first homology arm comprises homology to a first sequence adjacent to the target locus and the second homology arm comprises homology to a second sequence adjacent to the target locus. In some embodiments, the first homology arm comprises homology to a sequence at a 5′ end of the target gene and the second homology arm comprises homology to a sequence at a 3′ end of the target gene. In some embodiments, the first homology arm or the second homology arm comprises homology to a portion of a 5′ UTR of the target gene. In some embodiments, the first homology arm or the second homology arm comprises homology to a portion of a 3′ UTR of the target gene. In some embodiments, the first homology arm or the second homology arm comprises homology to a portion that is 5′ of a start codon of the target gene. In some embodiments, the first homology arm comprises homology to a portion of a 3′ UTR of the target gene and the second homology arm comprises homology to a portion that is 5′ of a transcription start site of the target gene.

In some embodiments, the first homology arm, the second homology arm, or both comprise at least about 200 base pairs. In some embodiments, the first homology arm, the second homology arm, or both comprise at least about 400 base pairs. In some embodiments, the first homology arm, the second homology arm, or both comprise at least about 500 base pairs. In some embodiments, the first homology arm, the second homology arm, or both comprise at least about 800 base pairs. In some embodiments, the first homology arm, the second homology arm, or both comprise at least about 850 base pairs. In some embodiments, the first homology arm, the second homology arm, or both comprise at least about 900 base pairs.

In some embodiments, the donor template comprises at least about 85%, sequence identity to SEQ ID NO:6. In some embodiments, the donor template comprises the sequence of SEQ ID NO:6. In some embodiments, expression of the integrated transgene is regulated by a promoter of the target gene. In some embodiments, the promoter is an endogenous promoter in a genome of the cell. In some embodiments, the introducing is performed ex vivo. In some embodiments, the programmable nuclease is a CRISPR-Cas protein. In some embodiments, the programmable nuclease is a Cas9 protein. In some embodiments, the programmable nuclease is a Cpf1 protein. In some embodiments, the programmable nuclease generates a double strand break at the target locus. In some embodiments, the donor template is introduced into the cell in a recombinant AAV (rAAV) vector. In some such embodiments, the rAAV vector is a AAV6 vector.

In some embodiments, the method further comprises introducing into the cell a guide RNA, wherein the guide RNA directs the programmable nuclease to cleave the target locus in the target gene. In some embodiments, the guide RNA comprises a sequence that hybridizes to a target sequence in the target gene. In some embodiments the guide RNA is any of the herein-described guide RNAs.

### BRIEF DESCRIPTION OF THE DRAWINGS

FIGS. 1A-1F: sgRNA & AAV6 design for CRISPR/AAV6-mediated targeting of the α-globin locus. FIG. 1A: Schematic of HBA2 and HBA1 genomic DNA. Sequence differences between the two genes in 3′ UTR region are depicted as red stars. Locations of the five prospective sgRNAs are indicated. FIG. 1B: Indel frequencies for each guide at both HBA2 and HBA1 in human CD34⁺ HSPCs are depicted in orange and blue, respectively. Bars represent median ±interquartile range. *: P<0.05; **: P<0.005; ***: P<0.0005 determined using unpaired t test. FIG. 1C: AAV6 DNA repair donor design schematics to introduce a SFFV-GFP-BGH integration are depicted at the HBA2 and HBA1 loci. FIG. 1D: Percentage of GFP⁺ cells using HBA2- and HBA1-specific guides and CS and WGR SFFV-GFP AAV6 donors as determined by flow cytometry. Bars represent median ±interquartile range. *: P<0.05 determined using unpaired t test. FIG. 1E: Targeted allele frequency at HBA2 and HBA1 as determined by ddPCR, to determine whether off-target integration occurs at the unintended gene. Bars represent median ±interquartile range. *: P<0.05; ***: P<0.0005 determined using unpaired t test. FIG. 1F: MFI of GFP⁺ cells across each targeting event as determined by BD FACSAria II platform. Bars represent median ±interquartile range. ***: P<0.0005 determined using unpaired t test.

FIGS. 2A-2F: CRISPR/AAV6-mediated targeting of the α-locus using a T2A scheme. FIG. 2A: AAV6 DNA repair donor design schematics to introduce a HBB-T2A-YFP integration are depicted at the HBA1 locus. FIG. 2B: Percentage of CD34⁻/CD45⁻ HSPCs that acquire RBC surface markers, GPA and CD71, as determined by flow cytometry. Bars represent median ±interquartile range. FIG. 2C: Percentage of GFP⁺ cells using HBA2- and HBA1-specific guides and HBB-T2A-YFP AAV6 donors as determined by flow cytometry. Bars represent median ±interquartile range. **: P<0.005 determined using unpaired t-test. FIG. 2D: Targeted allele frequency at HBA2 and HBA1 as determined by ddPCR. Bars represent median ±interquartile range. ***: P<0.0005 determined using unpaired t test. FIG. 2E: MFI of GFP⁺ cells across each targeting event as determined by BD FACSAria II platform. Bars represent median ±interquartile range. FIG. 2F: Representative flow cytometry staining and gating scheme for human HSPCs targeted at HBA1 with HBB-T2A-YFP (HBA1 UTRs) and differentiated into RBCs over the course of a 14-day protocol. This indicates that only RBCs (CD34⁻/CD45⁻/CD71⁺/GPA⁺) are able to express the integrated T2A-YFP marker. Analysis was performed on BD FACS Aria II platform.

FIGS. 3A-3F: CRISPR/AAV6-mediated targeting at the α-globin locus in SCD HSPCs. FIG. 3A: AAV6 DNA repair donor design schematics to introduce a whole gene replacement HBB transgene integration at the HBA1 locus. FIG. 3B: Percentage of CD34⁻/CD45⁻ HSPCs that acquire RBC surface markers, GPA and CD71, as determined by flow cytometry. Bars represent median ±interquartile range. FIG. 3C: Targeted allele frequency at HBA1 as determined by ddPCR. Bars represent median ±interquartile range. *: P<0.05 determined using unpaired t test. FIG. 3D: Representative HPLC plots for each treatment following targeting and RBC differentiation of human SCD CD34⁺ HSPCs. Retention time for HgbA and HgbS tetramer peaks are indicated at ˜6.6 and ˜9.8, respectively. FIG. 3E: Summary of all HPLC results showing percentage of HgbA out of total hemoglobin tetramers. Bars represent median ±interquartile range. *: P<0.05 determined using unpaired t test. FIG. 3F: Plot depicting correlation between % HgbA vs. % targeted alleles in HBA1 UTR-targeted samples that were differentiated into RBCs and analyzed by HPLC. Colors of respective vectors are as depicted in figure. R2 value and trendline formula are indicated.

FIGS. 4A-4F: Engraftment of α-globin-targeted human HSPCs into NSG mice. FIG. 4A: 16 weeks after bone marrow transplantation of targeted human CD34⁺ HSPCs into NSG mice, bone marrow was harvested and rates of engraftment were determined. Depicted is percentage of mTerr119⁻ cells (non-RBCs) that were hHLA⁺ from the total number of cells that were either mCd45⁺ of hHLA⁺. Indicated by color coding are large, medium, and small dose experiments where 1.2M, 750K, or 250K cells were initially transplanted, respectively. Bars represent median ±interquartile range. FIG. 4B: Among engrafted human cells, the distribution among CD19⁺ (B-cell), CD33⁺ (myeloid), or other (i.e., HSPC/RBC/T/NK/Pre-B) lineages are indicated. Bars represent median ±interquartile range. FIG. 4C: Targeted allele frequency at HBA1 as determined by ddPCR among in vitro (pre-transplantation) targeted HSPCs and bulk engrafted HSPCs as well as among CD19⁺ (B-cell), CD33+(myeloid), and CD34⁺ (HSC) lineages. Bars represent median ±interquartile range. FIG. 4D: Targeted allele frequency at HBA1 among engrafted human cells compared to the bulk targeting rate of the pre-transplantation, in vitro human HSPC population. Each mouse is represented by a different color. Bars represent median ±interquartile range. FIG. 4E: Following primary engraftments, engrafted human cells were transplanted a second time into the bone marrow of NSG mice. 16 weeks post-transplantation, bone marrow was harvested and rates of engraftment were determined. Depicted is the percentage of mTerr119⁻ cells (non-RBCs) that were hHLA⁺ from the total number of cells that were either mCd45⁺ or hHLA⁺. Bars represent median ±interquartile range. FIG. 4F: Targeted allele frequency at HBA1 as determined by ddPCR among engrafted human cells in bulk sample as well as among CD19⁺ (B-cell) and CD33⁺ (myeloid) lineages in secondary transplantation experiments. Each mouse is represented by a different color. Bars represent median ±interquartile range.

FIGS. 5A-5E: Targeting the α-globin locus in β-thalassemia-derived HSPCs. FIG. 5A: Targeted allele frequency at HBA1 in β-thalassemia-derived HSPCs as determined by ddPCR. Bars represent median ±interquartile range. *: P<0.05 determined using unpaired t test. FIG. 5B: Following differentiation of targeted HSPCs into RBCs, mRNA was harvested and converted into cDNA. Expression of HBA (does not distinguish between HBA1 and HBA2) and HBB transgene were normalized to GPA expression. FIG. 5C: 16 weeks after bone marrow transplantation of targeted β-thalassemia-derived HSPCs into NSG mice, bone marrow was harvested and rates of engraftment were determined. Depicted is percentage of mTerr119⁻ cells (non-RBCs) that were hHLA⁺ from the total number of cells that were either mCd45+ or hHLA⁺. Bars represent median ±interquartile range. FIG. 5D: Among engrafted human cells, the distribution among B-cell, myeloid, or other (i.e., HSPC/RBC/T/NK/Pre-B) lineages are indicated. Bars represent median ±interquartile range. FIG. 5E: Targeted allele frequency at HBA1 as determined by ddPCR among engrafted human cells in bulk sample as well as among CD19⁺ (B-cell), CD33⁺ (myeloid), and other (i.e., HSPC/RBC/T/NK/Pre-B) lineages in secondary transplantation experiments. Each mouse is represented by a different color. Bars represent median ±interquartile range.

FIGS. 6A-6C: Expected outcomes of introducing HBB transgene at endogenous locus. FIG. 6A: Expected outcome when integrating an undiverged, full-length HBB (with introns) at the endogenous locus of HSPCs derived from patients with β-thalassemia. The varieties of disease-causing mutations are annotated in the figure. FIG. 6B: Expected outcome when integrating a diverged, full-length HBB (with introns) at the endogenous locus of HSPCs derived from patients with β-thalassemia. FIG. 6C: Expected outcome when integrating a diverged, HBB cDNA (without introns) at the endogenous locus of HSPCs derived from patients with β-thalassemia.

FIGS. 7A-7C: Analysis of Cas9 sgRNAs targeting α-globin locus. FIG. 7A: Table with guide RNA sequences. PAM shown in gray, and differences between HBA1 and HBA2 are highlighted in red for each guide. Figure discloses SEQ ID NOS 40-44, respectively, in order of appearance. FIG. 7B: Summary of rhAmpSeq targeted sequencing results at on-target and 40 most highly-predicted off-target sites by COSMID for HBA1 sg5. Values are indel frequency for RNP treatment after subtraction of indel frequency for Mock treatment at each locus for each experimental replicate. N=3, though not all values are displayed since some were <0.01% after subtraction of Mock indel frequencies. Bars represent median. FIG. 7C: List of genomic coordinates for 40 most highly-predicted off-target sites by COSMID for HBA1 sg5. Figure discloses SEQ ID NOS 45-85, respectively, in order of appearance.

FIGS. 8A-8B: Targeting HSPCs with GFP-HBA integration vectors. FIG. 8A: Timeline for editing and analysis of HSPCs targeted with GFP-HBA integration vectors.

FIG. 8B: Depicted are representative flow cytometry images for human HSPCs that have been targeted by CRISPR/AAV6 methodology 14d post-editing. This indicates that whole-gene-replacement (WGR) integration yields a greater MFI per GFP⁺ cell than cut-site (CS) integration at the HBA1 locus. Analysis was performed on BD Accuri C6 platform. Median MFI across all replicates is shown below each flow cytometry image, and schematics of integration vectors are shown above.

FIG. 9: Timeline for targeting HSPCs with HBB-T2A-YFP-HBA integration vectors. Timeline for targeting of HSPCs with HBB-T2A-YFP integration vectors, differentiation into RBCs, and subsequent analysis.

FIGS. 10A-10B: Representative staining and gating scheme used to analyze targeting and differentiation rates of RBCs. FIG. 10A: Representative flow cytometry staining and gating scheme for human HSPCs targeted at HBA1 with HBB-T2A-YFP (HBA1 UTRs) and differentiated into RBCs over the course of a 14-day protocol. This indicates that only RBCs (CD34⁻*CD45⁻/CD71⁺/GPA⁺) are able to express the integrated T2A-YFP marker. Analysis was performed on BD FACS Aria II platform. FIG. 10B: Representative YFP×FSC flow cytometry images of RBCs (CD34⁻/CD45⁻/CD71⁺/GPA⁺) derived from HSPCs targeted with HBA1 UTRs, HBA2 UTRs, and HBB UTRs vector. AAV only controls were used for each vector to establish gating scheme, leading to slight variation in positive/negative cut-offs across images.

FIGS. 11A-11E: Analysis of colony-forming units of HSPCs plated into methylcellulose. FIG. 11A: Distribution of genotypes of methylcellulose colonies displayed in FIGS. 11B and 11D. Numbers of clones corresponding to each category are included in the pie chart. FIG. 11B: In vitro (pre-engraftment) live CD34⁺ HSPCs from healthy donors were single-cell sorted into 96-well plates containing semisolid methylcellulose media for colony forming assays. 14d post-sorting cells were analyzed for morphology. Depicted are number of colonies formed for each lineage (CFU-E=erythroid lineage; CFU-GEMM=multi-lineage; or CFU-GM=granulocyte/macrophage lineage) divided by the total number of wells available for colonies. FIG. 11C: Percent distribution of each lineage among all colonies for each treatment for FIG. 11A. FIG. 11D: In vitro (pre-engraftment) live CD34⁺β-thalassemia patient-derived HSPCs were single-cell sorted into 96-well plates containing semisolid methylcellulose media for colony forming assays. 14d post-sorting cells were analyzed for morphology. Depicted are number of colonies formed for each lineage (B=BFU-E and C=CFU-E (erythroid lineage); GE=CFU-GEMM (multi-lineage); or GM=CFU-GM (granulocyte/macrophage lineage)) divided by the total number of wells available for colonies. FIG. 11E: Percent distribution of each lineage among all colonies for each treatment for FIG. 11C.

FIG. 12: Integration cassettes screened for development of clinical vector. Displayed are schematics and corresponding rationale for design as well as eventual outcomes for Vectors S1-15.

FIG. 13: Timeline for targeting of HSPCs and transplantation into mice. Timeline for targeting of HSPCs with HBB integration vectors, transplantation into mice (both 1o and 2o engraftment), and subsequent analysis.

FIG. 14: Representative staining and gating scheme used to analyze engraftment and targeting rates of human HSPCs into NSG mice. Representative flow cytometry staining and gating scheme used to analyze targeting and engraftment rates of human HSPCs transplanted into the bone marrow of NSG mice. This sample was targeted with a UbC-GFP integration at the HBA1 locus. This demonstrates that only human cells (hHLA⁺) are able to express GFP. Analysis was performed on BD FACS Aria II platform.

FIGS. 15A-15G: Engraftment of human HSPCs targeted with GFP at α-globin locus into NSG mice. FIG. 15A: Timeline for targeting of HSPCs with UbC-GFP integration vector, transplantation into mice (both 1o and 2o engraftment), and subsequent analysis. FIG. 15B: AAV6 DNA repair donor design schematic to introduce a UbC-GFP-BGH integration is depicted at the HBA1 locus. FIG. 15C: 16 weeks after bone marrow transplantation of targeted human CD34⁺ HSPCs into NSG mice, bone marrow was harvested and rates of engraftment were determined (1o). Depicted is the percentage of mTerr119⁻ cells (non-RBCs) that were hHLA⁺ from the total number of cells that were either mCd45⁺ or hHLA⁺. Bars represent median ±interquartile range. FIG. 15D: Among engrafted human cells, the distribution among CD19⁺ (B-cell), CD33⁺ (myeloid), or other (i.e., HSPC/RBC/T/NK/Pre-B) lineages are indicated. Bars represent median ±interquartile range. FIG. 15E: Percentage of GFP⁺ cells among pre-transplantation (in vitro, post-sorting) and successfully-engrafted populations, both bulk HSPCs and among CD19⁺ (B-cell), CD33⁺ (myeloid), and other lineages. Bars represent median ±interquartile range. FIG. 15F: Following primary engraftments, engrafted human cells were transplanted a second time into the bone marrow of NSG mice. 16 weeks post-transplantation, bone marrow was harvested and rates of engraftment were determined (2o). Depicted is the percentage of mTerr119⁻ cells (non-RBCs) that were hHLA⁺ from the total number of cells that were either mCd45⁺ or hHLA⁺. FIG. 15G: Percentage of GFP⁺ cells among successfully-engrafted population from the secondary transplant depicted in FIG. 15F.

FIGS. 16A-16G. Targeting, β-globin production, and engraftment data in β-thalassemia patient-derived HSPCs. FIG. 16A: Percentage of CD34⁻/CD45⁻ HSPCs that acquire RBC surface markers, GPA and CD71, as determined by flow cytometry. Bars represent median ±interquartile range. N=4 for each treatment group. FIG. 16B: Targeted allele frequency at HBA1 in β-thalassemia-derived HSPCs as determined by ddPCR. Bars represent median ±interquartile range. N=3 for mock, N=2 for RNP only and HBA1 UTRs, and N=5 for HBA1 UTRs long HAs treatments. **: P<0.005 determined using unpaired t test. FIG. 16C: Following differentiation of targeted HSPCs into RBCs, mRNA was harvested and converted into cDNA. Expression of HBA (does not distinguish between HBA1 and HBA2) and HBB transgene were normalized to HBG expression. Bars represent median ±interquartile range. N=3 for each treatment group with the exception of HBA1 UTRs with N=1. **: P<0.05 determined using unpaired t test. FIG. 16D: Summary of hemoglobin tetramer HPLC results showing HgbA normalized to HgbF. Bars represent median ±interquartile range. N>3 for each treatment group. ***: P<0.0001 determined using unpaired t test. FIG. 16E: Representative hemoglobin tetramer HPLC plots for each treatment following targeting and RBC differentiation of HSPCs. Retention time for HgbF and HgbA tetramer peaks are indicated. FIG. 16F: Summary of reverse-phase globin chain HPLC results showing area under the curve (AUC) of β-globin/AUC of α-globin. Bars represent median ±interquartile range. N>4 for each treatment group. ***: P<0.0001 determined using unpaired t test. FIG. 16G: Representative reverse-phase globin chain HPLC plots for each treatment following targeting and RBC differentiation of HSPCs. Retention time for HgbF and HgbA tetramer peaks are indicated.

FIGS. 17A-17C. Targeting, β-globin production, and engraftment data in β-thalassemia patient-derived HSPCs. FIG. 17A: 16 weeks after bone marrow transplantation of targeted β-thalassemia-derived HSPCs into NSG mice, bone marrow was harvested and rates of engraftment were determined. Depicted is percentage of mTerr119⁻ cells (non-RBCs) that were hHLA⁺ from the total number of cells that were either mCd45⁺ or hHLA⁺. Bars represent median ±interquartile range. N=10. FIG. 17B: Among engrafted human cells, the distribution among B-cell, myeloid, or other (i.e., HSPC/RBC/T/NK/Pre-B) lineages are indicated. Bars represent median ±interquartile range. N=9. FIG. 17C: Targeted allele frequency at HBA1 as determined by ddPCR among engrafted human cells in bulk sample as well as among CD19⁺ (B-cell), CD33⁺ (myeloid), and other (i.e., HSPC/RBC/T/NK/Pre-B) lineages in secondary transplantation experiments. Bars represent median ±interquartile range. N=3 for mock treatment group and N=10 for targeted treatment group.

FIGS. 18A-18B: Additional information on the indel spectrum generated by the HBA1-targeting gRNA 5. FIG. 18A: Schematic depicting locations of all five guide sequences at genomic loci. Figure discloses SEQ ID NOS 86-87, respectively, in order of appearance. FIG. 18B: Representative indel spectrum of HBA1-specific sg5 generated by TIDE software.

FIG. 19: Viability data post-targeting in HSPCs. HSPC viability was quantified 2-4d post-editing by flow cytometry. Depicted are the percentage of cells that stained negative for GhostRed viability dye. All cells were edited with our optimized HBB gene replacement vector using standard conditions (i.e., electroporation of Cas9 RNP+sg5, 5K MOI of AAV, and no AAV wash at 24 h). Bars represent median ±interquartile range. WT: N=5 for mock, N=3 for RNP only, N=1 for AAV only, and N=6 for RNP+AAV treatment group; SCD: N=2 for each treatment group with the exception of RNP+AAV with N=4; N=3 for mock, N=1 for RNP only, and N=7 for RNP+AAV treatment group.

FIGS. 20A-20C. Data generated by dual-color targeting vectors to gain insight into mono- and bi-allelic editing frequencies when targeting HBA1. FIG. 20A: Representative FACS plots of CD34⁺ HSPCs simultaneously targeted by HBA1-WGR-GFP AAV6 (shown in FIG. 16C) and HBA1-WGR-mPlum AAV6. FIG. 20B: Table showing % of populations targeted with GFP only, mPlum only, and both colors. Percent of edited cells was then converted to % edited alleles by the following equation: (total % targeted cells+(% dual color)*2)/2=total % targeted alleles. FIG. 20C: Percent edited cells is plotted against % edited alleles for data shown in FIG. 20B. A polynomial regression (R²=0.9981) was used to determine an equation to convert between the % edited alleles to % edited cells and vice versa.

FIGS. 21A-21G. Updated data for custom transgene integration at HBA1 for red blood cell delivery. FIG. 21A: Percentage of CD34⁻/CD45⁻ HSPCs that acquire RBC surface markers, GPA and CD71, as determined by flow cytometry. Bars represent median ±interquartile range. N=5 for each treatment group. FIG. 21B: Targeted allele frequency at HBA1 in primary HSPCs as determined by ddPCR. Bars represent median ±interquartile range. N=3 for each treatment group. FIG. 21C: FIX (Factor IX) production in cell lysate and supernatant following targeting and red blood cell differentiation in primary HSPCs as determined by FIX ELISA. FIG. 21D: Production of tyrosine as a proxy for PAH activity in supernatant of 293T cells that were electroporated with transgene-expressing plasmids. FIG. 21E: % RBCs of primary HSPCs targeted at HBA1 with constitutive GFP and promoterless YFP integration vectors during the course of RBC differentiation as determined by flow cytometry. FIG. 21F: % GFP of targeted HSPCs shown in FIG. 21E as determined by flow cytometry. FIG. 21G: MFI fold change over d0 measurement of GFP⁺ population shown in FIG. 21F as determined by flow cytometry.

### DETAILED DESCRIPTION

### 1. Introduction

The present disclosure provides methods and compositions for integrating transgenes, e.g., for therapeutic genes such as HBB, IDUA, PAH, PDGFB, FIX (e.g., the Factor IX Padua variant), LDLR, and others, into the HBA1 or HBA2 locus in hematopoietic stem and progenitor cells (HSPCs).

The present methods can be used to introduce transgenes, e.g., coding sequences with optional elements such as promoters or other regulatory elements (e.g., enhancers, repressor domains), introns, WPREs, poly A regions, UTRs (e.g. 3′ UTRs), specifically into the HBA1 or HBA2 locus of HSPCs. In particular, the present disclosure provides guide RNA sequences that specifically recognize HBA1 but not HBA2, or HBA2 but not HBA1, enabling the selective cleavage of either HBA1 or HBA2 by an RNA-directed nuclease such as Cas9. By cleaving HBA1 or HBA2, but not both, in the presence of a donor template comprising a transgene, the transgene can integrate into the genome at the site of cleavage by homology directed recombination (HDR), e.g., replacing the endogenous HBA1 or HBA2 gene.

In particular embodiments, the present methods can be used to deliver an HBB transgene into HBA1, which could be used as a universal treatment strategy for patients with β-thalassemia, regardless of which mutations in HBB are responsible for the disease. In particular, integration at this locus is able to produce high levels of functional transgene, capable of forming adult hemoglobin tetramers. It is also possible to use site-specific integration at this locus for RBC-mediated delivery of other therapeutically relevant transgenes.

### 2. General

Practicing this disclosure utilizes routine techniques in the field of molecular biology. Basic texts disclosing the general methods of use in this disclosure include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of an HBB cDNA, transgene, or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” refers to a polynucleotide sequence that can be used to repair a double stranded break (DSB) in the DNA, e.g., a CRISPR/Cas9-mediated break at the HBA1 or HBA2 locus as induced using the herein-described methods and compositions. The homologous repair template comprises homology to the genomic sequence surrounding the DSB, i.e., comprising HBA1 or HBA2 homology arms. In some embodiments, two distinct homologous regions are present on the template, with each region comprising at least 50, 100, 200, 300, 400, 500, 600, 700, 800, 900 or more nucleotides or more of homology with the corresponding genomic sequence. In particular embodiments, the templates comprise two homology arms comprising about 500 nucleotides of homology extending from either site of the sgRNA target site. The repair template can be present in any form, e.g., on a plasmid that is introduced into the cell, as a free floating doubled stranded DNA template (e.g., a template that is liberated from a plasmid in the cell), or as single stranded DNA. In particular embodiments, the template is present within a viral vector, e.g., an adeno-associated viral vector such as AAV6. The templates of the present disclosure can also comprise a transgene, e.g., HBB transgene.

HBA1 and HBA2 (hemoglobin subunit alpha 1 and 2, respectively) are closely related, but not identical, genes encoding alpha-globin, which is a component of hemoglobin. HBA1 and HBA2 are located within the alpha-globin locus, located on human chromosome 16. Their coding sequences are identical, but the genes diverge, e.g., in the 5′UTRs, introns, and particularly the 3′UTRs. The NCBI gene ID for HBA1 is 3039, and the NCBI gene ID for HBA2 is 3040, the entire disclosure of which are herein incorporated by reference.

HBB (hemoglobin subunit beta) is a gene encoding the beta subunit of hemoglobin, which in normal adults comprises two alpha chains and two beta chains. Mutations in HBB, e.g., causing a reduction or absence of HBB expression or function, can cause β-thalassemia. The NCBI gene ID No. for human HBB is 3043, and the UniProt ID is P68871, the entire disclosures of which are herein incorporated by reference.

As used herein, “homologous recombination” or “HR” refers to insertion of a nucleotide sequence during repair of double-strand breaks in DNA via homology-directed repair mechanisms. This process uses a “donor template” or “homologous repair template” with homology to nucleotide sequence in the region of the break as a template for repairing a double-strand break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-strand break, such as meganucleases, such as zinc finger nucleases (ZFNs), transcription activator-like effector nucleases (TALENs), and the CRISPR-Cas9 gene editing systems. In particular embodiments, HR involves double-stranded breaks induced by CRISPR-Cas9.

### 4. CRISPR/Cas Systems Specifically Targeting the HBA1 or HBA2 Locus

The present disclosure is based in part on the identification of CRISPR guide sequences that specifically direct the cleavage of HBA1 or HBA2 by RNA-guided nucleases but without leading to cleavage of both genes. The present disclosure provides a CRISPR/AAV6-mediated genome editing method that can achieve high rates of targeted integration at both loci. The integrated transgenes exhibit RBC-specific expression of functional transgenes, and cells edited at this locus are capable of long-term engraftment and hematopoietic reconstitution.

Because of the redundancy of HBA1 and HBA2, integration at this locus allows delivery of transgenes for RBC-specific expression without the risk of bi-allelic integrations causing detrimental cellular effects. Furthermore, in the treatment of β-thalassemia, because the pathology is caused both by lack of HBB as well as aggregation of unpaired alpha-globin, knocking HBB into HBA1 addresses both problems in a single genome editing event, allowing the simultaneous increase of HBB levels and decrease of levels of alpha-globin. Attempts have also been made to introduce b-like globin transgenes at alpha-globin loci, but these approaches can give rise to large numbers of potential genetic events upon editing, including the generation of large deletions, inversions, or other deleterious rearrangements.

sgRNAs

The single guide RNAs (sgRNAs) of the present disclosure target the HBA1 or HBA2 locus. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the HBA1 or HBA2 locus, and a constant region that mediates binding to Cas9 or another RNA-guided nuclease. The sgRNA can target any sequence within HBA1 or HBA2 adjacent to a PAM sequence. In particular embodiments, the sgRNA targets a sequence within either the HBA1 or HBA2 gene, but not within both genes, i.e., the sgRNA targets a sequence within HBA1 or HBA2 that is distinct between the two genes and that is adjacent to a PAM sequence. In particular embodiments, the sgRNA targets HBA1 but does not target HBA2 (e.g., it specifically binds to and/or leads to the cleavage of HBA1 but not HBA2, and/or its target sequence is 100% identical to a sequence within HBA1 but is not 100% identical to a sequence within HBA2). In some such embodiments, the sgRNA targets the sequence of SEQ ID NO:5. In particular embodiments, the sgRNA targets HBA2 but does not target HBA1 (e.g., it specifically binds to and/or leads to the cleavage of HBA2 but not HBA1, and/or its target sequence is 100% identical to a sequence within HBA2 but is not 100% identical to a sequence within HBA1). In some such embodiments, the sgRNA targets the sequence of SEQ ID NO:2. In particular embodiments, a single guide RNA, or sgRNA, is used. In some embodiments, the target sequence is within intron 2 or the 3′ UTR of HBA1 or HBA2. In particular embodiments, the target sequence is within the 3′ UTR of HBA1 or HBA2. In particular embodiments, the target sequence differs by 3, 4, 5 or more nucleotides between HBA1 and HBA2. In some embodiments, the target sequence comprises one of the sequences shown as SEQ ID NOS:1-5, or a sequence comprising 1, 2, 3 or more mismatches with one of SEQ ID NOS:1-5. In particular embodiments, the target sequence comprises the target sequence of sg2 (SEQ ID NO:2) or sg5 (SEQ ID NO:5). In some embodiments, the sgRNA targets a sequence within the HBA1 or HBA2 gene (i.e., within the coding sequence, 5′UTR, an intron, or 3′UTR), but does not target a sequence in the intergenic region between the HBA1 and HBA2 genes. In some embodiments, the sgRNA only targets a single site within the genome.

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA as described herein and being guided to and cleaving the specific HBA1 or HBA2 sequence targeted by the targeting sequence of the sgRNA. In particular embodiments, the Cas9 is from Streptococcus pyogenes.

Also disclosed herein are CRISPR/Cas or CRISPR/Cpf1 systems that target and cleave DNA at the HBA1 or HBA2 locus. An exemplary CRISPR/Cas system comprises (a) a Cas (e.g., Cas9) or Cpf1 polypeptide or a nucleic acid encoding said polypeptide, and (b) an sgRNA that hybridizes specifically to HBA1 or HBA2, or a nucleic acid encoding said guide RNA. In some instances, the nuclease systems described herein, further comprises a donor template as described herein. In particular embodiments, the CRISPR/Cas system comprises an RNP comprising an sgRNA targeting HBA1 or HBA2 and a Cas protein such as Cas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the HBA1 or HBA2 locus to carry out the methods disclosed herein.

Introducing the sgRNA and Cas Protein into Cells

The guide RNA and nuclease can be introduced into the cell using any suitable method, e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In particular embodiments, the guide RNA and nuclease are assembled into ribonucleoproteins (RNPs) prior to delivery to the cells, and the RNPs are introduced into the cell by, e.g., electroporation.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with β-thalassemia. In such embodiments, the transgene that is integrated into the genome of the HSPC is HBB, e.g., at the HBA1 locus. In one embodiment, a method is provided of treating a subject with β-thalassemia, comprising genetically modifying a plurality of HSPCs isolated from the subject so as to integrate the HBB gene at the HBA1 locus, and reintroducing the HSPCs into the subject. In some such embodiments, HSPCs differentiate into red blood cells (RBCs) in vivo, and the RBCs express higher levels of beta-globin, and lower levels of alpha-globin, as compared to the levels in RBCs from the subject that have not been subjected to the present methods.

To avoid immune rejection of the modified cells when administered to a subject, the cells to be modified are preferably derived from the subject's own cells. Thus, preferably the mammalian cells are autologous cells from the subject to be treated with the modified cells.

In some embodiments, cells are harvested from the subject and modified according to the methods disclosed herein, which can include selecting certain cell types, optionally expanding the cells and optionally culturing the cells, and which can additionally include selecting cells that contain the transgene integrated into the HBA1 or HBA2 locus. In particular embodiments, such modified cells are then reintroduced into the subject.

Further disclosed herein are methods of using said nuclease systems to produce the modified host cells described herein, comprising introducing into the cell (a) an RNP of the present disclosure that targets and cleaves DNA at the HBA1 or HBA2 locus, and (b) a homologous donor template or vector as described herein. Each component can be introduced into the cell directly or can be expressed in the cell by introducing a nucleic acid encoding the components of said one or more nuclease systems.

Such methods will target integration of the functional transgene, e.g., HBB transgene, at the endogenous HBA1 or HBA2 locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the HBA1 or HBA2 locus, and (b) a homologous donor template or vector as described herein.

In any of these methods, the nuclease can produce one or more single stranded breaks within the HBA1 or HBA2 locus, or a double-stranded break within the HBA1 or HBA2 locus. In these methods, the HBA1 or HBA2 locus is modified by homologous recombination with said donor template or vector to result in insertion of the transgene into the locus. The methods can further comprise (c) selecting cells that contain the transgene integrated into the HBA1 or HBA2 locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an mRNA encoding i53 can be introduced into the cell, e.g., by electroporation at the same time as an sgRNA-Cas9 RNP. The sequence of i53 can be found, inter alia, at www.addgene.org/92170/sequences/.

Techniques for the insertion of transgenes, including large transgenes, capable of expressing functional proteins, including enzymes, cytokines, antibodies, and cell surface receptors are known in the art (See, e.g. Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

Transgene Integration with Reduced Off-Target Effects, Inversions, and/or Translocations

In an aspect, provided herein are methods for reducing random integration of donor templates for introduction of exogenous nucleic acids in a target genome. Off-target or random integration of donor templates can occur when double stranded breaks are created by endogenous or exogenous DNA cleavage mechanisms, e.g., a nuclease, where the cleavage is not at the intended genomic sequence. Off-target or random integration may result in unintended increase or decrease in expression of genes in the target genome, and may have deleterious impact. In some embodiments, a guide RNA used herein specifically binds to one target sequence in the target genome, thereby reducing off-target binding and cleaving of the target genome. In some embodiments, programmable nuclease, e.g. a Cas nuclease directed by a guide RNA, or a zinc finger protein or TALEN protein provided herein specifically binds to and results in cleavage of a single specific target sequence in a target genome. In some embodiments, the target gene may belong to a gene family or a gene locus that comprises multiple genes that share high sequence similarity. For example, a guide RNA used herein may target a HBA1 or a HBA2 gene. In some embodiments, a guide RNA used herein specifically hybridizes to a target sequence in a HBA1 gene or a HBA2 gene, but not both. In some embodiments, the guide RNA specifically hybridizes to a 3′ UTR sequence of a HBA1 gene. In some embodiments, the guide RNA specifically hybridizes to a 3′ UTR sequence of a HBA2 gene. In some embodiments, the guide RNA specifically hybridizes to a 5′ UTR sequence of a HBA1 gene. In some embodiments, the guide RNA specifically hybridizes to a 5′ UTR sequence of a HBA2 gene. In some embodiments, a guide RNA specifically hybridizing to a target sequence in a HBA1 or a HBA2 gene results in reduced off-target cleavage in a host genome as compared to a guide RNA that hybridizes with a target sequence in both a HBA1 and a HBA2 gene. In some embodiments, a guide RNA specifically hybridizing to a target sequence in a HBA1 or a HBA2 gene results in reduced off-target integration of a DNA donor template in a host genome as compared to a guide RNA that hybridizes with a target sequence in both a HBA1 and a HBA2 gene. In some embodiments, a guide RNA specifically hybridizing to a target sequence in a HBA1 or a HBA2 gene does not result in off-target integration of a DNA donor template in a host genome. In some embodiments, a guide RNA specifically hybridizing to a target sequence in a HBA1 or a HBA2 gene results in reduced off-target integration of a DNA donor template in a host genome as compared to a guide RNA that hybridizes with a target sequence in both a HBA1 and a HBA2 gene by at least 1%, 2%, 3%, 4%, 5%, 6%, 7%, 8%, 9%, 10%, 11%, 12%, 13%, 14%, 15%, 20%, 25%, 30%, 35%, 40%, 50%, 75%, 90%, 95, or 99%.

Chromosomal translocations joins NDA segments in a genome derived from two heterologous regions or chromosomes. Translocation events can occur from improper repair of double stranded breaks (DSBs), including DSBs generated by nucleases such as a Cas9 nuclease. In an aspect, provided herein are methods and compositions for integration of one or more transgenes into a target genome with reduced translocation events. For example, a guide RNA provided herein can direct a programmable nuclease, e.g. a Cas9, to generate a double stranded break at one particular locus of a target genome. Without wishing to be bound by any theory, a guide RNA or a programmable nuclease specifically targeting a single target sequence or a single target locus allows for specific cleavage at the target sequence. In some embodiments, the target gene belongs to a gene family or a gene locus that comprises multiple genes that share high sequence similarity. For example, a guide RNA used herein may target a HBA1 or a HBA2 gene. In some embodiments, a guide RNA used herein specifically hybridizes to a target sequence in a HBA1 gene or a HBA2 gene, but not both. In some embodiments, the guide RNA specifically hybridizes to a 3′ UTR sequence of a HBA1 gene. In some embodiments, the guide RNA specifically hybridizes to a 3′ UTR sequence of a HBA2 gene. In some embodiments, the guide RNA specifically hybridizes to a 5′ UTR sequence of a HBA1 gene. In some embodiments, the guide RNA specifically hybridizes to a 5′ UTR sequence of a HBA2 gene. In some embodiments, a guide RNA specifically hybridizing to a target sequence in a HBA1 or a HBA2 gene results in a single cleavage event in the target genome. In some embodiments, the guide RNA directs a programmable nuclease to create a cleavage in a HBA1 gene sequence and not in a HBA2 gene sequence. In some embodiments, the guide RNA directs a programmable nuclease to create a cleavage in a HBA2 gene sequence and not in a HBA1 gene sequence. In some embodiments, a guide RNA specifically hybridizing to a target sequence in a HBA1 or a HBA2 gene results in reduced translocation or inversion events in the target genome as compared to a guide RNA that hybridizes with a target sequence in both a HBA1 and a HBA2 gene. In some embodiments, a guide RNA specifically hybridizing to a target sequence in a HBA1 and not a HBA2 gene, a donor template and a RNA guided programmable nuclease are introduced in a population of cells. In some embodiments, after the introduction, the population of cells only comprise three integration outcomes at the HBA1 or HBA2 gene sequence: 1) no integration, 2) indel created in a HBA1 sequence and not a HBA2 sequence, and 3) integration of the donor template that replaces the HBA1 sequence. In some embodiments, after the introduction, the population of cells do not comprise any of the following integration outcomes at the HBA1 or HBA2 gene sequence: 1) indel in a HBA2 sequence, 2) indels in both a HBA1 and a HBA2 sequence, 3) deletion of both the HBA1 and the HBA2 sequence, 4) integration of the donor template that replaces the HBA2 sequence, 5) deletion of the HBA2 sequence, 6) integration in the HBA1 sequence and indel in the HBA2 sequence, 7) integration in the HBA2 sequence and indel in the HBA1 sequence, 8) inversion of the target genome region containing the HBA1 and HBA2 gene sequence, or 9) chromosomal translocation.

In some embodiments, a guide RNA specifically hybridizing to a target sequence in a HBA2 and not a HBA1 gene, a donor template and a RNA guided programmable nuclease are introduced in a population of cells. In some embodiments, after the introduction, the population of cells only comprise three integration outcomes at the HBA1 or HBA2 gene sequence: 1) no integration, 2) indel created in a HBA2 sequence and not a HBA1 sequence, and 3) integration of the donor template that replaces the HBA2 sequence. In some embodiments, after the introduction, the population of cells do not comprise any of the following integration outcomes at the HBA1 or HBA2 gene sequence: 1) indel in a HBA1 sequence, 2) indels in both a HBA1 and a HBA2 sequence, 3) deletion of both the HBA1 and the HBA2 sequence, 4) integration of the donor template that replaces the HBA1 sequence, 5) deletion of the HBA1 sequence, 6) integration in the HBA1 sequence and indel in the HBA2 sequence, 7) integration in the HBA2 sequence and indel in the HBA1 sequence, 8) inversion of the target genome region containing the HBA1 and HBA2 gene sequence, or 9) chromosomal translocation. In some embodiments, a programmable nuclease specifically targeting one target sequence in the target genome, e.g., a Cas9 directed by a gRNA specifically hybridizes to a HBA1 sequence or a HBA2 sequence but not both in the target genome results in reduced translocation events as compared to a Cas9 directed by a gRNA that hybridizes to both a HBA1 sequence and a HBA2 sequence. In some embodiments, the frequency of translocation events is reduced by at least 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 3.5-fold, 4-fold, 4.5-fold, 5-fold, 6-fold, 7-fold, 8-fold, 9-fold, 10-fold, or more than 10-fold. In some embodiments, a programmable nuclease specifically targeting one target sequence in the target genome, e.g., a Cas9 directed by a gRNA specifically hybridizes to a HBA1 sequence or a HBA2 sequence but not both and a donor template are introduced into a population of cells, e.g. HSPC cells. In some embodiments, the introduction results in translocation events in less than 10% of the population of cells. In some embodiments, the introduction results in translocation events in less than 50% of the population of cells. In some embodiments, the introduction results in translocation events in less than 5% of the population of cells. In some embodiments, the introduction results in translocation events in less than 5%, less than 10%, less than 15%, less than 20%, less than 25%, less than 30%, less than 35%, less than 40%, less than 45%, less than 50%, less than 55%, or less than 60% of the population of cells. In some embodiments, the introduction results in translocation events in less than 1% of the population of cells. In some embodiments, the introduction results in translocation events in less than 0.5% of the population of cells. In some embodiments, the introduction results in translocation events in less than 0.1% of the population of cells. In some embodiments, the introduction results in translocation events that is not detectable in the population of cells as compared with a reference or control population of cells, wherein the reference cell population is introduced with, e.g. the programmable nuclease and no guide RNA.

Translocation events may be detected by standard TaqMan assay for DNA quantification in which PCR is performed in conjunction with a probe that releases a fluorophore upon annealing to DNA and subsequent degradation by the DNA polymerase. In the intact probe the fluorophore signal is suppressed via interaction with covalently attached quenchers. The probe is designed to anneal inside the region that is being amplified by the PCR primers. The fluorescent signal detected is thus proportional to the amount of amplicon present in the sample. Methods for detection of translocation as described in Burman et al., Genome Biology 16, 146 (2015) is incorporated herein by reference in its entirety.

In another aspect, the disclosure provides a population of cells having alterations at two or more target nucleic acids made using any method disclosed herein, wherein the population of cells has a translocation frequency of less than 5%. In one embodiment, the translocation frequency is less than 4%. In one embodiment, the translocation frequency is less than 3%. In one embodiment, the translocation frequency is less than 2%. In one embodiment, the translocation frequency is less than 1%. In one embodiment, the translocation frequency is less than 0.5%. In one embodiment, the translocation frequency is less than 0.25%. In one embodiment, the translocation frequency is less than 0.1%. In one embodiment, the population of cells comprises a translocation frequency that is lower than a translocation frequency of a reference cell population, wherein the reference cell population is introduced with, e.g. the programmable nuclease and no guide RNA.

### Homologous Repair Templates

The transgene to be integrated, which is comprised by a polynucleotide or donor construct, can be any transgene whose gene product is desirable in red blood cells. For example, the transgene could be used to replace or compensate for a defective gene, e.g., a defective HBB gene in a subject with β-thalassemia. In other embodiments, the transgene could express a secreted protein that provides a potential therapeutic benefit in a subject, such that genetically modified HSPCs can be introduced into a subject and differentiate into red blood cells, and the red blood cells then circulate and secrete the encoded protein in vivo. An exemplary, non-limiting list of suitable transgenes includes PDFGB (Platelet-derived growth factor subunit B; see, e.g., NCBI Gene ID No. 5155), IDUA (alpha-L-iduronidase; see, e.g., NCBI Gene ID No. 3425), PAH (phenylalanine hydroxylase; see, e.g., NCBI Gene ID No. 5053), Factor IX (or FIX; see, e.g., NCBI Gene ID NO. 2158), including Hyperactive Factor IX Padua, or the Padua Variant (see, e.g., Simioni et al., (2009) NEJM 361:1671-1675; Cantore et al. (2012) Blood 120:4517-4520; Monahan et al., (2015) Hum. Gene. Ther. 26:69-81), LDLR (low density lipoprotein receptor; see, e.g., NCBI Gene ID No. 3949), and others.

The transgene comprises a functional coding sequence for a gene, e.g., a gene that is defective in a subject, with optional elements such as promoters or other regulatory elements (e.g., enhancers, repressor domains), introns, WPREs, poly A regions, UTRs (e.g. 3′ UTRs).

In some embodiments, the transgene in the homologous repair template comprises or is derived from a cDNA for the corresponding gene. In some embodiments, the transgene in the homologous repair template comprises the coding sequence from the corresponding gene and one or more introns. In some embodiments, the transgene in the homologous repair template is codon-optimized, e.g., comprises at least 70%, 75%, 80%, 85%, 90%, 95%, or more homology to the corresponding wild-type coding sequence or cDNA, or a fragment thereof.

In particular embodiments, the template further comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the coding sequence and the 5′ end of the polyA sequence, so as to increase the expression of the transgene. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference).

To facilitate homologous recombination, the transgene is flanked within the polynucleotide or donor construct by sequences homologous to the target genomic sequence. For example, the transgene can be flanked by sequences surrounding the site of cleavage as defined by the guide RNA. In particular embodiments, the transgene is flanked by sequences homologous to the 3′ and to the 5′ ends of the HBA1 or HBA2 gene or coding sequence, such that the HBA1 or HBA2 gene is replaced upon the HDR-mediated integration of the transgene. In one such embodiment, the transgene is flanked on one side by a sequence corresponding to the 3′ UTR of the HBA1 or HBA2 gene, and on the other side by a sequence corresponding to the region of the transcription start site, e.g., just 5′ of the start site, of HBA1 or HBA2. The homology regions can be of any size, e.g., 100-1000 bp, 300-800 bp, 400-600 bp, or about 100, 200, 300, 400, 500, 600, 700, 800, 900, 1000, or more bp. In some embodiments, the transgene comprises a promoter, e.g., a constitutive or inducible promoter, such that the promoter drives the expression of the transgene in vivo. In particular embodiments, the transgene replaces the coding sequence of HBA1 or HBA2 such that its expression is driven by the endogenous HBA1 or HBA2 promoter. In particular embodiments, the donor template comprises a sequence comprising at least about 70%, 75%, 80%, 85%, 90%, 95%, 96%, 97%, 98%, 99%, or more identity to SEQ ID NO:6, or a fragment thereof. In particular embodiments, the donor template comprises the sequence shown as SEQ ID NO:6, or a fragment thereof.

As described herein, a transgene for introduction into a target sequence in a target genome may be a polynucleotide encoding a protein or a portion or fragment thereof, a polynucleotide comprising a regulatory sequence of a gene, an untranslated region of a gene, a promoter, an enhancer, an intron, an exon, an expression cassette, an expression tag, or any combination thereof. In some embodiments, a transgene (or a polynucleotide for insert, e.g. a coding sequence or a fragment thereof, a regulatory sequence, an intron, an exon, an expression cassette, or a tag, for example, a fluorescence tag) is flanked by one or more homology arms that have sequence homology or identity to nucleic acid sequences in the target genome. For example, a transgene may be flanked by a first homology arm and/or a second homology arm on either 5′ or 3′ end of the transgene. In some embodiments, the first homology arm and/or the second homology arm comprises sequences homologous to the 3′ end and/or the 5′ end of a target gene. For example, a transgene may be flanked by a 5′ homology arm and a 3′ homology arm, where the 5′ homology arm is homologous to a 5′ flanking sequence of the target gene, or where the 3′ homology arm is homologous to a 3′ flanking sequence of the target gene. In some embodiments, the transgene is flanked by a 5′ homology arm and a 3′ homology arm, where the 5′ homology arm is homologous to a 5′ flanking sequence of the target gene, and the 3′ homology arm is homologous to a 3′ flanking sequence of the target gene. In some embodiments, the transgene is flanked by a 5′ homology arm and a 3′ homology arm, where the 5′ homology arm is homologous to a 5′ UTR sequence of the target gene and/or where the 3′ homology arm is homologous to a 3′ UTR sequence of the target gene. In some embodiments, the transgene is flanked by a 5′ homology arm and a 3′ homology arm, where the 5′ homology arm is homologous to a sequence 5′ to a 5′ UTR sequence of the target gene and/or where the 3′ homology arm is homologous to a sequence 3′ to a 3′ UTR sequence of the target gene. In some embodiments, the transgene is flanked by a 5′ homology arm and a 3′ homology arm, where the 5′ homology arm is homologous to a sequence immediately 5′ to a 5′ UTR sequence of the target gene and/or where the 3′ homology arm is homologous to a sequence immediately 3′ to a 3′ UTR sequence of the target gene. In some embodiments, the transgene is flanked by a 5′ homology arm and a 3′ homology arm, where the 5′ homology arm is homologous to a sequence 5′ to a 5′ terminus of a coding region of the target gene and/or where the 3′ homology arm is homologous to a sequence 3′ to a 3′ terminus of a coding region of the target gene. In some embodiments, the transgene is flanked by a 5′ homology arm and a 3′ homology arm, where the 5′ homology arm is homologous to a sequence immediately 5′ to a 5′ terminus of a coding region of the target gene and/or where the 3′ homology arm is homologous to a sequence immediately 3′ to a 3′ terminus of a coding region of the target gene. In some embodiments, the transgene is flanked by a 5′ homology arm and a 3′ homology arm, where the 5′ homology arm is homologous to a sequence 5′ to a 5′ terminus of a open reading frame of the target gene and/or where the 3′ homology arm is homologous to a sequence 3′ to a 3′ terminus of an open reading frame of the target gene. In some embodiments, the transgene is flanked by a 5′ homology arm and a 3′ homology arm, where the 5′ homology arm is homologous to a sequence immediately 5′ to a 5′ terminus of a open reading frame of the target gene and/or where the 3′ homology arm is homologous to a sequence immediately 3′ to a 3′ terminus of an open reading frame of the target gene. As used herein, an open reading frame refers to a reading frame of a gene that has the ability of being transcribed into a precursor mRNA and/or a protein. An ORF can start with a start codon (e.g. ATG) and end with a stop codon (e.g. UAA). In some embodiments, the protein is translated from the ORF a full length and/or functional protein.

In some embodiments, the transgene is flanked by a 5′ homology arm and a 3′ homology arm, where the 5′ homology arm is homologous to a sequence 5′ to a 5′ terminus of the whole coding sequence of the target gene and/or where the 3′ homology arm is homologous to a sequence 3′ to a 3′ terminus of the whole coding sequence of the target gene. In some embodiments, the transgene is flanked by a 5′ homology arm and a 3′ homology arm, where the 5′ homology arm is homologous to a sequence immediately 5′ to a 5′ terminus of the whole coding sequence of the target gene and/or where the 3′ homology arm is homologous to a sequence immediately 3′ to a 3′ terminus of the whole coding sequence of the target gene. In some embodiments, the transgene is flanked by a 5′ homology arm, where the 5′ homology arm is homologous to a sequence 5′ to a transcription initiation start site of the target gene. In some embodiments, the transgene is flanked by a 5′ homology arm, where the 5′ homology arm is homologous to a sequence immediately 5′ to a transcription initiation start site of the target gene. In some embodiments, the transgene is flanked by a 5′ homology arm, where the 5′ homology arm is homologous to a sequence 5′ to a first exon of the target gene. In some embodiments, the transgene is flanked by a 5′ homology arm, where the 5′ homology arm is homologous to a sequence immediately 5′ to a first exon of the target gene. In some embodiments, the transgene is flanked by a 5′ homology arm, where the 5′ homology arm is homologous to a sequence 5′ to a first intron of the target gene. In some embodiments, the transgene is flanked by a 5′ homology arm, where the 5′ homology arm is homologous to a sequence immediately 5′ to a first intron of the target gene. In some embodiments, the transgene is flanked by a 5′ homology arm, where the 5′ homology arm is homologous to a sequence 5′ to a last intron of the target gene. In some embodiments, the transgene is flanked by a 5′ homology arm, where the 5′ homology arm is homologous to a sequence immediately 5′ to a last intron of the target gene. In some embodiments, the transgene is flanked by a 5′ homology arm, where the 5′ homology arm is homologous to a sequence 5′ to a last intron of the target gene. In some embodiments, the transgene is flanked by a 5′ homology arm, where the 5′ homology arm is homologous to a sequence 5′ to a last intron of the target gene. In some embodiments, the transgene is flanked by a 5′ homology arm, where the 5′ homology arm is homologous to a sequence 5′ to a last exon of the target gene. In some embodiments, the transgene is flanked by a 5′ homology arm, where the 5′ homology arm is homologous to a sequence immediately 5′ to a last exon of the target gene. In some embodiments, the transgene is flanked by a 5′ homology arm, where the 5′ homology arm is homologous to a sequence 5′ to a last exon of the target gene. In some embodiments, the transgene is flanked by a 5′ homology arm, where the 5′ homology arm is homologous to a sequence 5′ to a last exon of the target gene.

In some embodiments, a part or a fragment of the target gene is replaced by the transgene. In some embodiments, the whole coding sequence of the target gene is replaced by the transgene. In some embodiments, the coding sequence and regulatory sequences of the transgene is replaced by the transgene. In some embodiments, the target gene sequence replaced by the transgene comprises an open reading frame. In some embodiments, the target gene sequence replaced by the transgene comprises an expression cassette. In some embodiments, the target gene sequence replace by the transgene comprises a sequence that transcribes into a precursor mRNA. In some embodiments, the target gene sequence replaced by the transgene comprises a 5′UTR, one or more introns, one or more exons, and a 3′ UTR.

Whole gene replacement may be performed with methods and compositions provided herein. When a nuclease, e.g., a Cas9 RNP introduces a cut into a desired gene, through the flanking homology sequences the whole gene may be replaced. In some embodiments, the target gene replaced belongs to the HBA locus. In some embodiments, the target gene replaced is HBA1 or HBA2. In some embodiments, the transgene comprises a polynucleotide encoding a reporter protein, e.g. a GFP. In some embodiments, the transgene comprises a polynucleotide encoding a HBB protein or a fragment thereof.

In some embodiments, the left homology arm is upstream of the cut site. In some embodiments, the left homology arm is downstream of the cut site. In some embodiments, the cut site is in a non-coding region. In some embodiments, the cut site is in a coding region. In some embodiments, the cut site is part of the untranslated region (UTR). In some embodiments, the cute site is at an intron.

In some embodiments, the 5′ homology arm is at least 100 bp, 200 bp, 300 bp, 400 bp, 500 bp, 600 bp, 700 bp, 800 bp, 900 bp, 1000 bp or more in length. In some embodiments, the, the 5′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 5′ homology arm is at least 400 bp in length. In some embodiments, the 5′ homology arm is at least 500 bp, 600 bp, 700bo, 800 bp, 900 bp, or 1000 bp in length. In some embodiments, the 5′ homology arm is at least 850 bp in length. In some embodiments, the 5′ homology arm is 400-500 bp. In some embodiments, the 5′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length.

In some embodiments, the 3′ homology arm is at least 100 bp, 200 bp, 300 bp, 400 bp, 500 bp, 600 bp, 700 bp, 800 bp, 900 bp, 1000 bp or more in length. In some embodiments, the, the 3′ homology arm is 100 bp, 150 bp, 200 bp, 250 bp, 275 bp, 300 bp, 325 bp, 350 bp, 375 bp, 400 bp, 450 bp, or greater than 500 bp in length. In some embodiments, the 3′ homology arm is at least 400 bp in length. In some embodiments, the 3′ homology arm is at least 500 bp, 600 bp, 700bo, 800 bp, 900 bp, or 1000 bp in length. In some embodiments, the 3′ homology arm is at least 850 bp in length. In some embodiments, the 3′ homology arm is 400-500 bp. In some embodiments, the 3′ homology arm is 400-500 bp, 400-550 bp, 400-600 bp, 400-650 bp, 400-700 bp, 400-750 bp, 400-800 bp, 400-850 bp, 400-900 bp, 400-950 bp, 400-1000 bp, 400-1100 bp, 400-1200 bp, 400-1300 bp, 400-1400 bp, 450-500 bp, 450-550 bp, 450-600 bp, 450-650 bp, 450-700 bp, 450-750 bp, 450-800 bp, 450-850 bp, 450-900 bp, 450-950 bp, 450-1000 bp, 450-1100 bp, 450-1200 bp, 450-1300 bp, 450-1450 bp, 500-600 bp, 500-650 bp, 500-700 bp, 500-750 bp, 500-800 bp, 500-850 bp, 500-900 bp, 500-950 bp, 500-1000 bp, 500-1100 bp, 500-1200 bp, 500-1300 bp, 500-1500 bp, 550-600 bp, 550-650 bp, 550-700 bp, 550-750 bp, 550-800 bp, 550-850 bp, 550-900 bp, 550-950 bp, 550-1000 bp, 550-1100 bp, 550-1200 bp, 550-1300 bp, 550-1500 bp, 600-650 bp, 600-700 bp, 600-750 bp, 600-800 bp, 600-850 bp, 600-900 bp, 600-950 bp, 600-1000 bp, 600-1100 bp, 600-1200 bp, 600-1300 bp, 600-1600 bp, 650-700 bp, 650-750 bp, 650-800 bp, 650-850 bp, 650-900 bp, 650-950 bp, 650-1000 bp, 650-1100 bp, 650-1200 bp, 650-1300 bp, 650-1500 bp, 700-700 bp, 700-750 bp, 700-800 bp, 700-850 bp, 700-900 bp, 700-950 bp, 700-1000 bp, 700-1100 bp, 700-1200 bp, 700-1300 bp, 700-1500 bp, 750-800 bp, 750-850 bp, 750-900 bp, 750-950 bp, 750-1000 bp, 750-1100 bp, 750-1200 bp, 750-1300 bp, 750-1500 bp, 800-850 bp, 800-900 bp, 800-950 bp, 800-1000 bp, 800-1100 bp, 800-1200 bp, 800-1300 bp, 800-1500 bp, 850-900 bp, 850-950 bp, 850-1000 bp, 850-1100 bp, 850-1200 bp, 850-1300 bp, 850-1500 bp, 900-950 bp, 900-1000 bp, 900-1100 bp, 900-1200 bp, 900-1300 bp, 900-1500 bp, 1000-1100 bp, 1100-1200 bp, 1200-1300 bp, 1300-1400 bp, or 1400-1500 bp in length.

Any suitable method can be used to introduce the polynucleotide, or donor construct, into the cell. In some instances, the donor template is single stranded, double stranded, a plasmid or a DNA fragment. In some instances, plasmids comprise elements necessary for replication, including a promoter and optionally a 3′ UTR. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector. In particular embodiments, the polynucleotide is introduced using a recombinant adeno-associated viral vector, e.g., rAAV6.

In some embodiments, the targeting construct comprises: (1) a viral vector backbone, e.g. an AAV backbone, to generate virus; (2) arms of homology to the target site of at least 200 bp but ideally at least 400 bp on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a functional protein and capable of expressing the functional protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

In some embodiments, viral vectors, e.g., AAV6 vector, is transduced at a multiplicity of infection (MOI) of, e.g., about 1×10³, 5×10³, 1×10⁴, 5×10⁴, 1×10⁵, between 2×10⁴ and 1×10⁵ viruses per cell, or less than 1×10⁵.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

In some embodiments, the donor template or vector comprises a nucleotide sequence homologous to a fragment of the HBA1 or HBA2 locus, or a nucleotide sequence is at least 85%, 88%, 90%, 92%, 95%, 98%, or 99% identical to at least 200, 250, 300, 350, 400, 450, 500, or more consecutive nucleotides of the HBA1 or HBA2 locus.

The inserted construct can also include other safety switches, such as a standard suicide gene into the locus (e.g. iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous HBA1 or HBA2 locus. In some embodiments, the present methods allow for the insertion of the donor template in 20%, 25%, 30%, 35%, 40%, or more cells, e.g., cells from an individual with β-thalassemia. The methods also allow for high levels of expression of the encoded protein in cells, e.g., cells from an individual with β-thalassemia, with an integrated transgene, e.g., levels of expression that are at least about 70%, 75%, 80%, 85%, 90%, 95%, or more relative to the expression in healthy control cells.

In some embodiments, the CRISPR-mediated systems as described herein (e.g., comprising a guide RNA, RNA-guided nuclease, and homologous repair template) are assessed in primary HSPCs, e.g., as derived from mobilized peripheral blood or from cord blood. In such embodiments, the HSPCs can be WT primary HSPCs (e.g., for initial testing of the system) or from patient-derived HSPCs (e.g., for pre-clinical in vitro testing).

### 5. Methods of Treatment

Following the integration of the transgene into the genome of the HSPC and confirming expression of the encoded therapeutic protein, a plurality of modified HSPCs can be reintroduced into the subject. In one embodiment, the HSPCs are introduced by intrafemoral injection, such that they can populate the bone marrow and differentiate into, e.g., red blood cells. In some embodiments, the HSPCs are induced to differentiate into red blood cells in vitro, and the modified red blood cells are then re-introduced into the subject.

Disclosed herein, in some embodiments, are methods of treating a genetic disorder, e.g., β-thalassemia in an individual in need thereof, the method comprising providing to the individual a protein replacement therapy using the genome modification methods disclosed herein. In some instances, the method comprises a modified host cell ex vivo, comprising a functional transgene, e.g., HBB transgene, integrated at the HBA1 or HBA2 locus, wherein the modified host cell expresses the encoded protein which is deficient in the individual, thereby treating the genetic disorder in the individual.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell as described herein is provided. The modified autologous host cell is genetically engineered to comprise an integrated transgene at the HBA1 or HBA2 locus. The modified host cell of the disclosure herein may be formulated using one or more excipients to, e.g.: (1) increase stability; (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8 (351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the disorder, e.g., β-thalassemia. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of β-thalassemia or other genetic disorder is also encompassed by the disclosure.

The present disclosure also contemplates kits comprising compositions or components of the present disclosure, e.g., sgRNA, Cas9, RNPs, i53, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

### 6. Examples

The present disclosure will be described in greater detail by way of specific examples. The following examples are offered for illustrative purposes only, and are not intended to limit the disclosure in any manner. Those of skill in the art will readily recognize a variety of noncritical parameters which can be changed or modified to yield essentially the same results.

### Example 1. Gene Replacement of α-Globin with β-Globin Restores Hemoglobin Balance in β-Thalassemia-Derived Hematopoietic Stem and Progenitor Cells

### Introduction

In this study, we have leveraged the combined Cas9/AAV6 genome editing method to mediate site-specific integration of a full-length HBB transgene into the HBA1 locus while leaving the virtually identical HBA2 gene unperturbed. We found that this process allowed us to replace the entire coding region of HBA1 with an HBB transgene at high frequencies, which both normalized the β-globin:α-globin imbalance in β-thalassemia-derived HSPCs and rescued functional adult hemoglobin tetramers in RBCs. Following transplantation experiments into immunodeficient NSG (non-obese diabetic scid gamma) mice, we found that edited HSCs were able to repopulate the hematopoietic system in vivo and could potentiate long-term engraftment, indicating that the editing process does not disrupt normal hematopoietic stem cell function.

The high efficiency of the gene replacement strategy suggests that the approach could be broadly applicable to a wide variety of monogenic diseases caused by loss-of-function mutations scattered throughout a particular gene, thus expanding the genome editing toolbox through a new way of using homologous recombination to precisely engineer the genome of therapeutically relevant human primary cells.

### Results

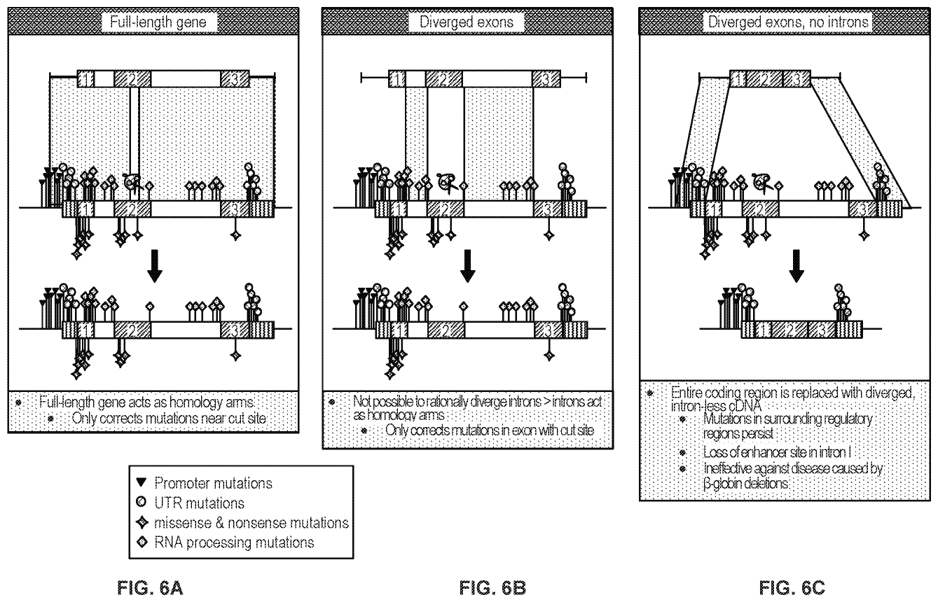

Cas9/AAV6-mediated genome editing is a robust system capable of introducing large genomic integrations at high frequencies across many loci in a wide variety of cell types, including HSPCs (19-22). In fact, this system has been successfully employed to correct the disease-causing mutation responsible for sickle cell disease (SCD) at the HBB locus at high frequencies in HSPCs (23). However, β-thalassemia is caused by loss-of-function mutations scattered throughout HBB, rather than the single polymorphism responsible for SCD. Therefore, a universal correction scheme for all patients requires delivery of a full-length copy of HBB. The simplest method for doing so would be to knock in a functional HBB transgene at the endogenous locus. However, this approach suffers from a number of technical issues: 1) Cas9-mediated DSBs in HBB could disrupt partially functional alleles in β-thalassemia minor and intermediate patients; 2) codon divergence is required to integrate a full-length β-globin cDNA at the endogenous locus in order to prevent partial recombination surrounding the Cas9 DSB site, which can negatively affect transgene expression levels (24); 3) introns must be removed since they cannot be rationally diverged, which may disrupt the important functional role that the HBB introns are known to play in gene regulation (25, 26); 4) disease-causing mutations in surrounding regulatory regions are likely to persist after integration of diverged cDNA; and 5) this strategy would be ineffective for many patients with disease caused by large deletions of the β-globin locus (FIG. 6) (27).

Prior work has shown that β-thalassemia patients with lowered α-globin levels demonstrate a less severe disease phenotype (5, 28). Therefore, knock-in of full-length HBB at the α-globin locus could most effectively allow us to improve the β-globin: α-globin imbalance in a single genome editing event while overcoming the problems inherent with introducing HBB into the endogenous locus.

### Efficient and Specific Indel Formation in the α-Globin Genes

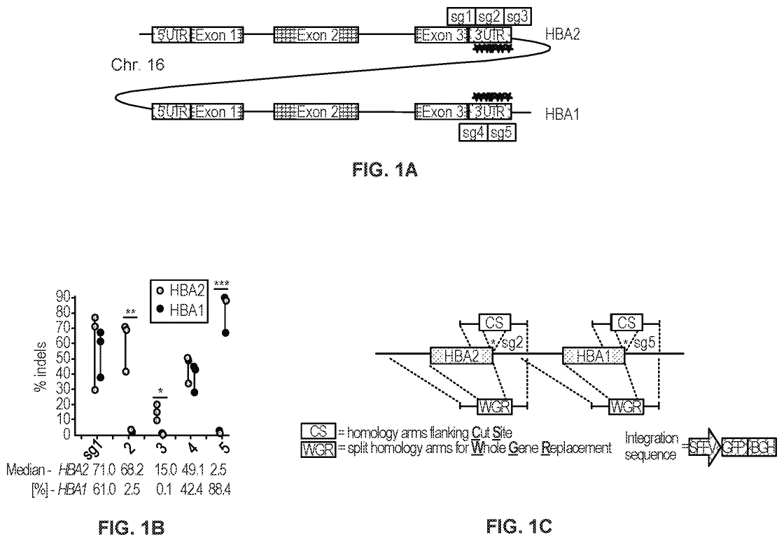

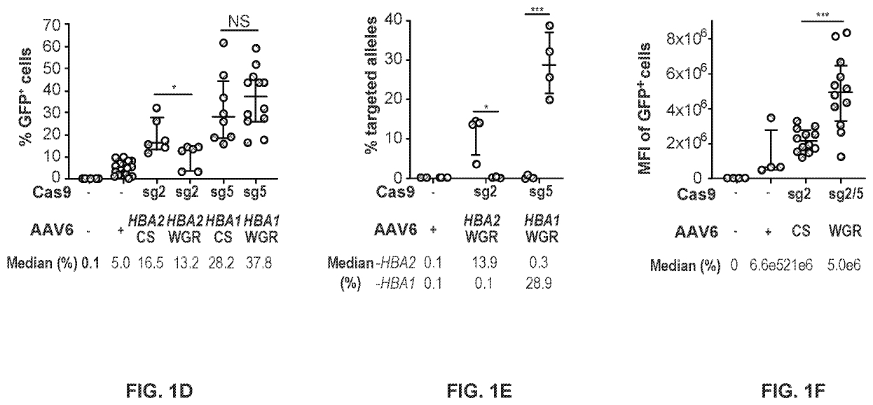

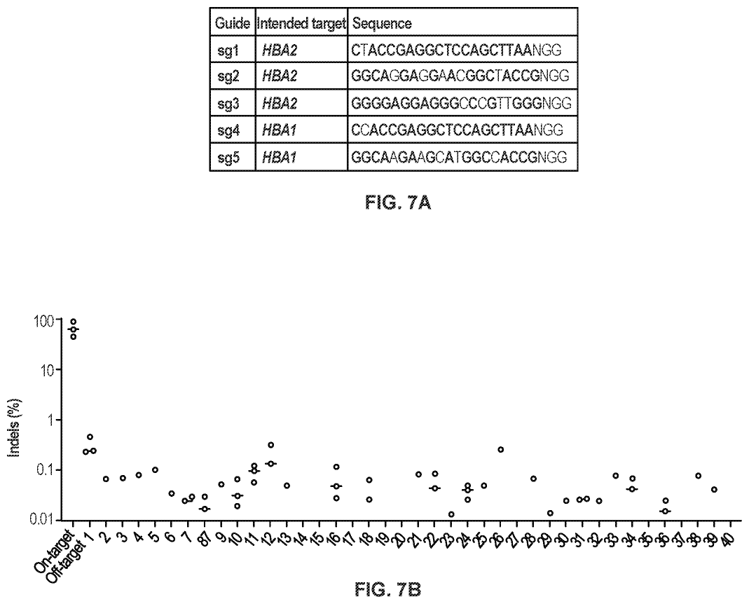

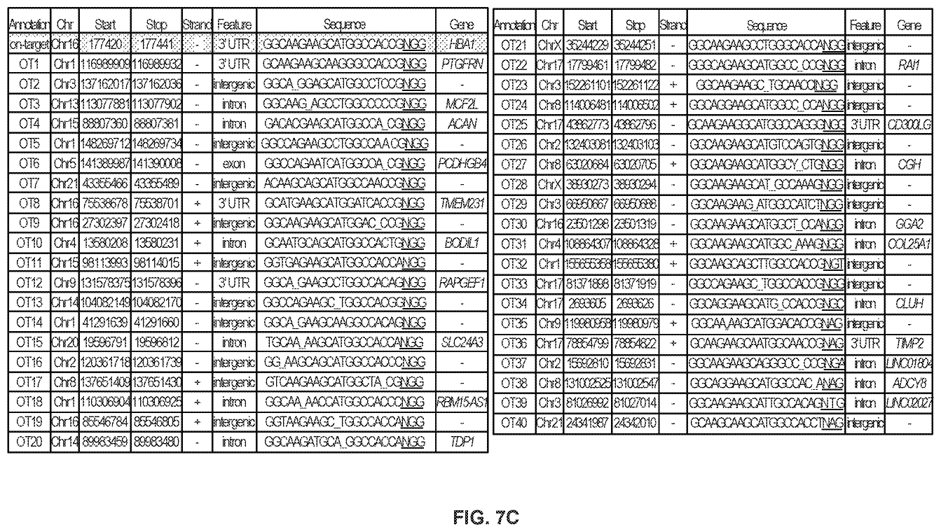

Because α-globin is expressed from two genes (HBA1 and HBA2) as HSPCs differentiate into RBCs, we hypothesized that site-specific integration of HBB into a single α-globin gene could allow us to achieve RBC-specific HBB expression without eliminating critical α-globin production. Though the HBA1 and HBA2 genes are virtually indistinguishable (5′ UTR, all three exons, and intron 1 are 100% identical; intron 2 is 94.0% identical; 3′ UTR is 83.8% identical), we were able to identify a limited number of CRISPR/Cas9 single guide RNA (sgRNA) sites (termed sg1-5; see, e.g., SEQ ID NOS: 1-5) that would be expected to facilitate cleavage of one α-globin gene and not the other (FIG. 1A). Screening for guides that distinguished between the two genes was critical as the Cas9/sgRNA ribonucleoprotein delivery system has become so effective in CD34⁺ HSPCs (>90% insertions/deletions (indels)) that we did not want to create four-gene-knockout α-thalassemia by having a sgRNA that was active at both α-globin genes. We therefore chose to test guides that exploited sequence differences between the two genes located within the 3′ UTR. To do so, we delivered each chemically-modified sgRNA (29) pre-complexed with Cas9 ribonucleoprotein (RNP) to human CD34⁺ HSPCs by electroporation in order to determine which of the five 3′ UTR guides can most efficiently and specifically induce indels (insertions/deletions) at the intended gene. We then PCR amplified the 3′ UTR regions of both HBA2 and HBA1 and analyzed the corresponding Sanger sequences for indel frequencies using TIDE analysis (30). We found that four of the five guides facilitate high frequency indel formation, two of which allowed for discrimination between HBA1 and HBA2 (sg2 cuts HBA2 and sg5 cuts HBA1) and two of which that did not (sg1 and sg4 cut both HBA2 and HBA1) (FIG. 1B). HBA1 and HBA2 target sequences for sg1 and sg4 only differed by one base pair at position 19 from the PAM (FIG. 7A), likely accounting for the lack of specificity. On the other hand, target sequences for the highly-specific sg2 and sg5 differed by five base pairs. Additionally, to determine the off-target activity of one of these sgRNAs, we electroporated cells with high-fidelity Cas9 (31) complexed with sg5 and then performed targeted sequencing of the 40 most likely off-target sites as predicted by COSMID (32). This determined that the HBA1-specific sg5 was extremely specific, with an average on-target activity of 66.6% and only two of the forty predicted sites showing activity above detection threshold (median of 0.31% and 0.14% activity at off-target sites 1 and 12, respectively) (FIG. 7B). These off-target sites are located in the 3′ UTR of genes PTGFRN and RAPGEF1, respectively, and because these regions are non-coding, the creation of small indels would be predicted to have no functional impact (FIG. 7C).

Upstream Homology Arm Strategy Allows Replacement of α-Globin with Custom Integration

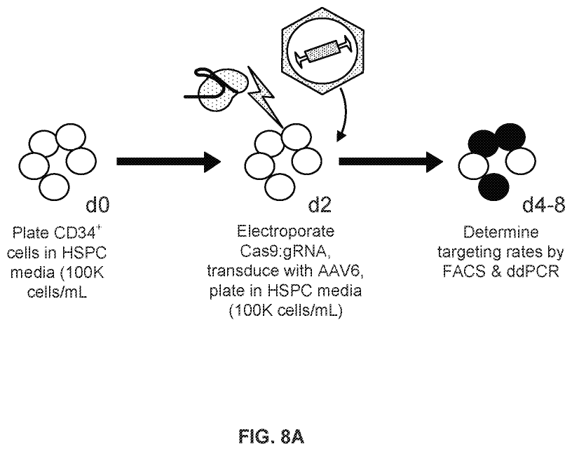

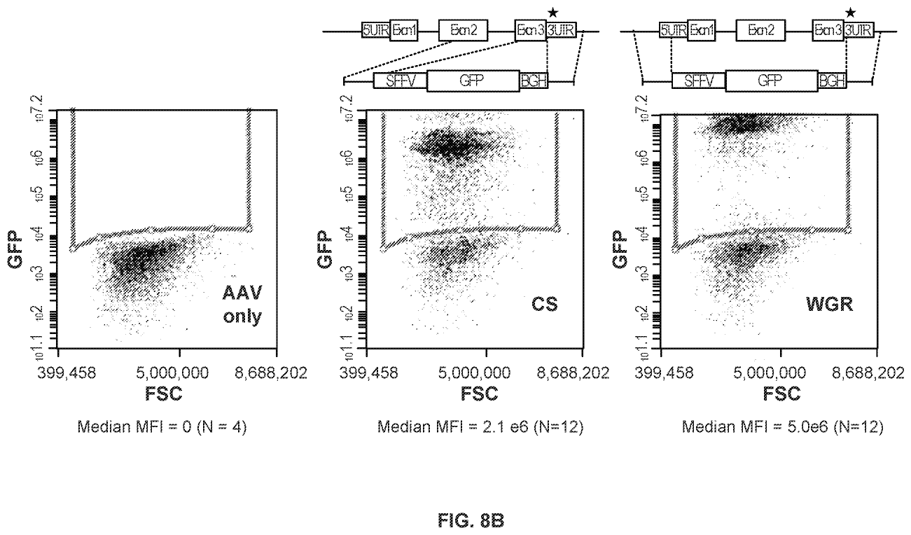

Upon identification of HBA1- and HBA2-specific guides, we tested the frequency of AAV6-mediated homologous recombination (HR) at these loci. We designed AAV6 donor template vectors that would integrate a GFP expression cassette directly into the sites of the Cas9 RNP-induced breaks. This was facilitated by 400 bp homology arms immediately flanking the cut site (hereafter termed “CS”) of each sgRNA (FIG. 1C). We delivered the most specific guides (sg2 to target HBA2 and sg5 to target HBA1) complexed with Cas9 RNP by electroporation into human CD34⁺ HSPCs. Since it had been previously reported that electroporation can aid AAV transduction (33), we added each AAV6 vector immediately following electroporation of Cas9 RNP in order to maximize AAV delivery. Several days later, after episomal expression in the “AAV only” controls was reduced, we analyzed integration frequencies by flow cytometry (FIG. 8A). As expected, we found that vectors with homology arms flanking the cut site efficiently integrated at both HBA2 and HBA1 in CD34⁺ HSPCs as determined by flow cytometry (median of 16.5% and 28.2% cells were GFP⁺, respectively) (FIG. 1D). However, because the cut site resides in the 3′ UTR for each gene, this approach would not ensure knockout of either α-globin gene upon HR. Therefore we also cloned repair template vectors with a left homology arm located upstream of the cut site, spanning the immediate 400 bp 5′ of the start codon of each gene. This approach exploits the HR repair process and could facilitate full replacement of the coding region of each α-globin gene, not only reducing α-globin production, but also allowing expression of the integrated transgene to be driven by the endogenous α-globin promoter (hereafter termed “WGR” for “whole gene replacement”) (FIG. 1C). The process of whole gene replacement was found to occur at a low but measurable frequency in T cells for engineering of the CD40L by TALENs (34). We found that having a left homology arm upstream of the cut site in the WGR strategy significantly reduced editing frequency at HBA2 (16.5% vs. 13.2%; P<0.05) (FIG. 1D). Surprisingly, this effect appeared to be gene-dependent, as the WGR strategy at HBA1 did not yield a coordinate decrease in editing frequency (28.2% vs. 37.8%) (FIG. 1D). Because the left homology arms were identical in each WGR vector, we used droplet digital PCR (ddPCR) to confirm that our integrations were specific only into the intended gene and correlated well with our targeting frequencies as determined by GFP expression (FIG. 1E). Notably, when performing flow cytometry on these cells, we found that the mean fluorescence intensity (MFI) of GFP⁺ cells was significantly higher for WGR vectors compared to CS vectors (P<0.0005) (FIG. 1F; FIG. 8B).

### Whole Gene Replacement at α-Globin Yields RBC-Specific Transgene Expression

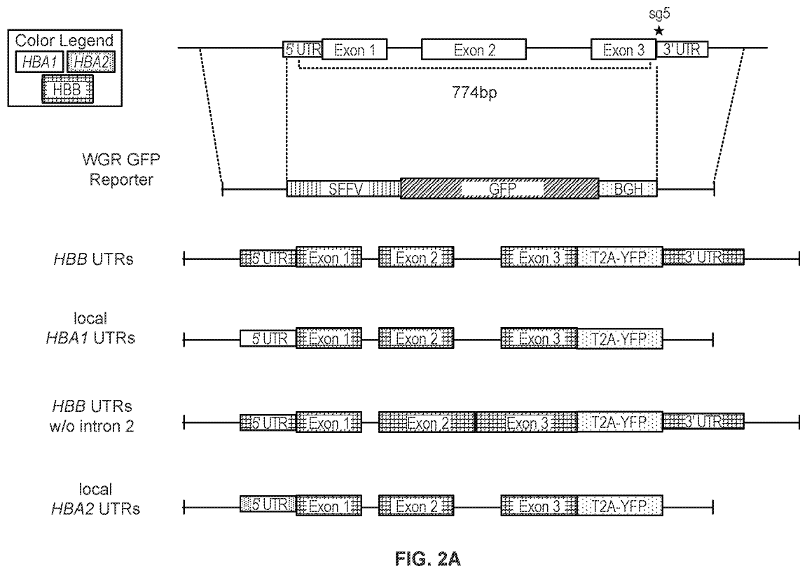

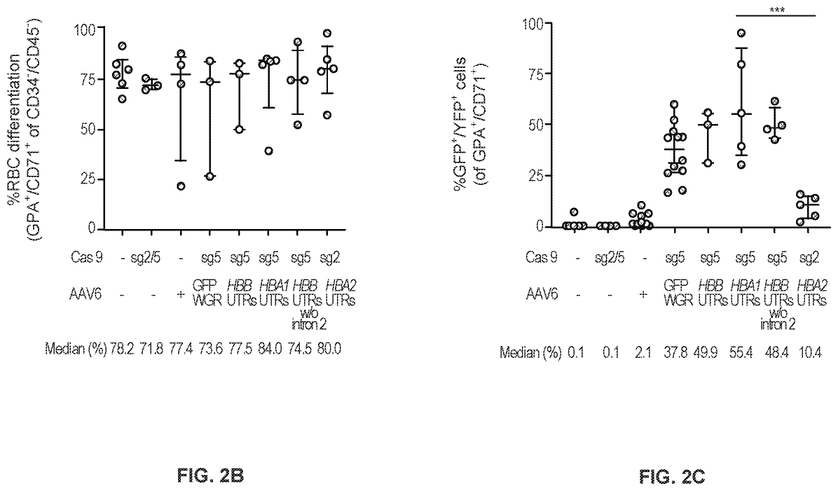

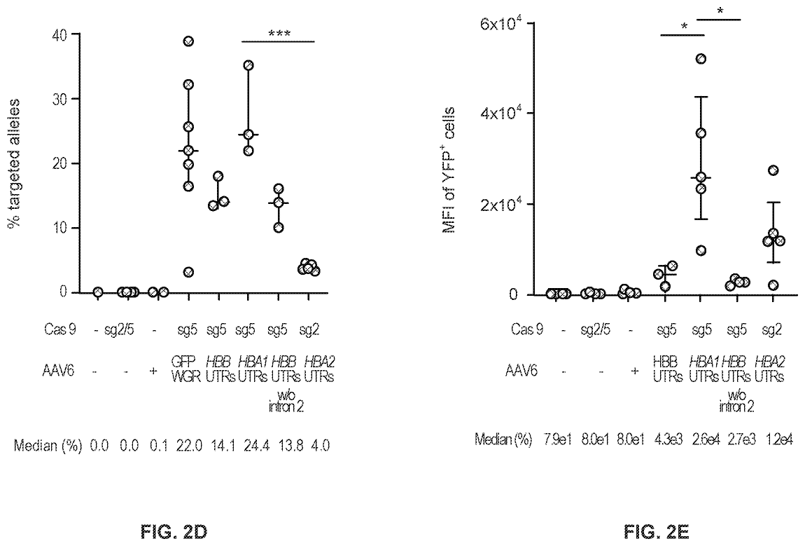

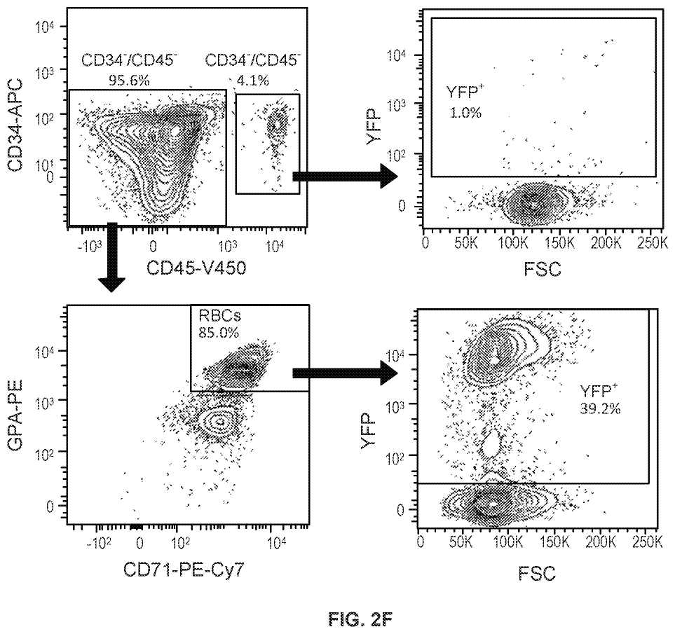

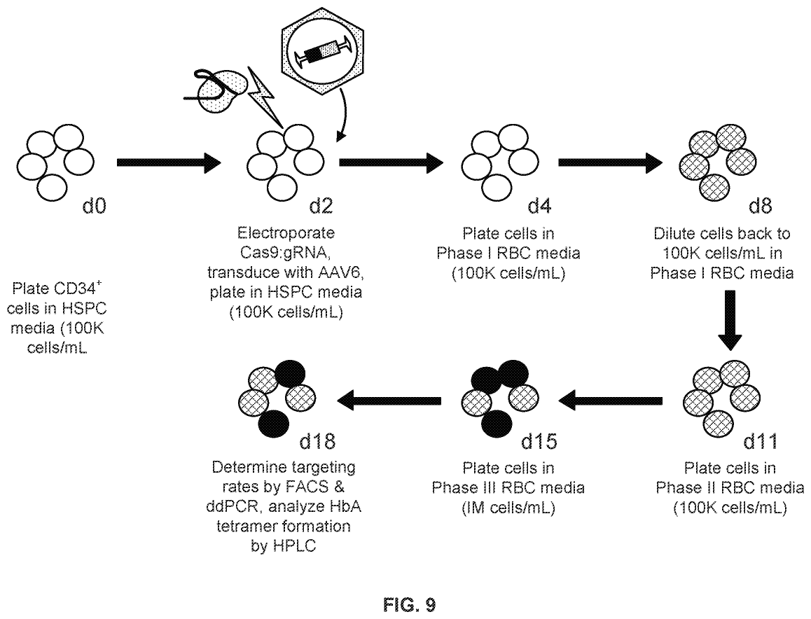

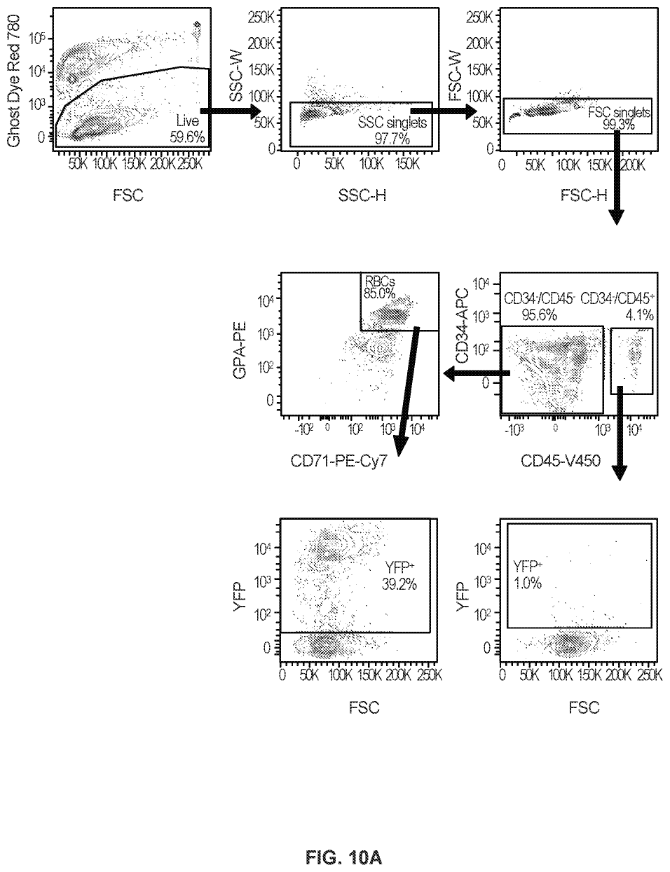

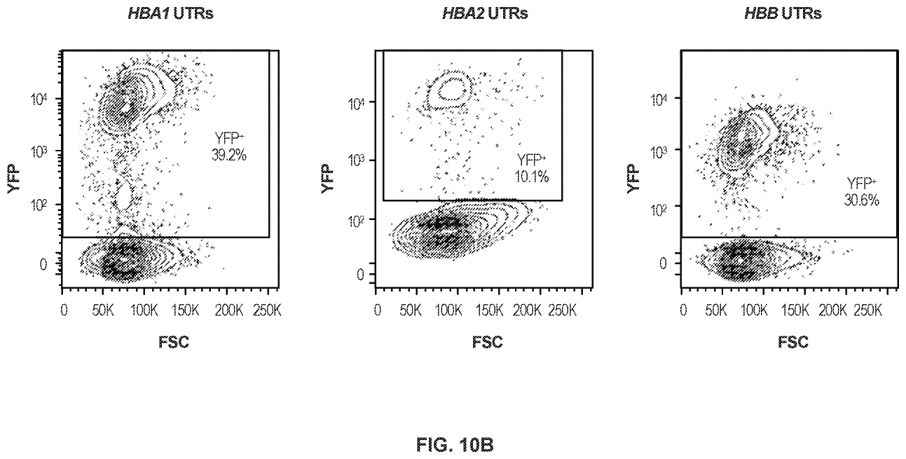

Due to the fact that the WGR repair template design at HBA1: 1) leads to equivalent integration frequencies compared to the CS design, 2) yields GFP expression levels that are greater than the CS design, and 3) ensures the knockout of one gene copy of α-globin, we next adapted this scheme to integrate a full-length HBB transgene at the HBA1 locus. To facilitate tracking of HBB transgene expression we fused it to a T2A-YFP sequence, which enables fluorescent readout of editing frequencies as a surrogate for HBB protein levels (FIG. 2A). To determine the significance of untranslated regions (UTRs) flanking the HBB-T2A-YFP cassette (either HBB UTRs or endogenous HBA1/2 UTRs), as well as the impact of removing the largest HBB intron (intron 2,850 bp), we designed multiple different AAV6 repair template vectors and analyzed integration frequencies and transgene expression. We targeted HSPCs as previously described, then differentiated cells into erythrocytes using an established protocol (35, 36) and determined integration frequencies and expression levels by flow cytometry (FIG. 9). We found that targeting HSPCs at HBA1 and HBA2 had no discernible effect on their ability to differentiate into RBCs compared to “Mock” (i.e., electroporation only), “RNP only”, and “AAV only” controls (FIG. 2B). Targeting frequencies were confirmed by both flow cytometry and ddPCR, allowing us to conclude that the vector with HBA1 UTRs integrated most efficiently (a mean of 55.4% of cells were YFP+, and a mean of 24.4% of total alleles were targeted) (FIGS. 2C-2D). Furthermore, we found that the MFI of YFP+ cells was significantly greater for the HBA1 UTR vector compared to either vector with HBB UTRs (P<0.05) (FIG. 2E; FIG. 10), potentially indicating higher HBB expression levels in the context of HBA1 regulatory regions. Because the HBB-T2A-YFP integration is driven by the endogenous promoter, we were able to determine that YFP was only expressed in GPA⁺/CD71⁺ RBCs (FIG. 2F), leading us to conclude that HBA1 is an effective safe harbor site for achieving RBC-specific expression, while leaving α-globin production from HBA2 unperturbed.

### Integration of HBB Transgene at HBA1 Locus Yields Adult Hemoglobin Tetramers

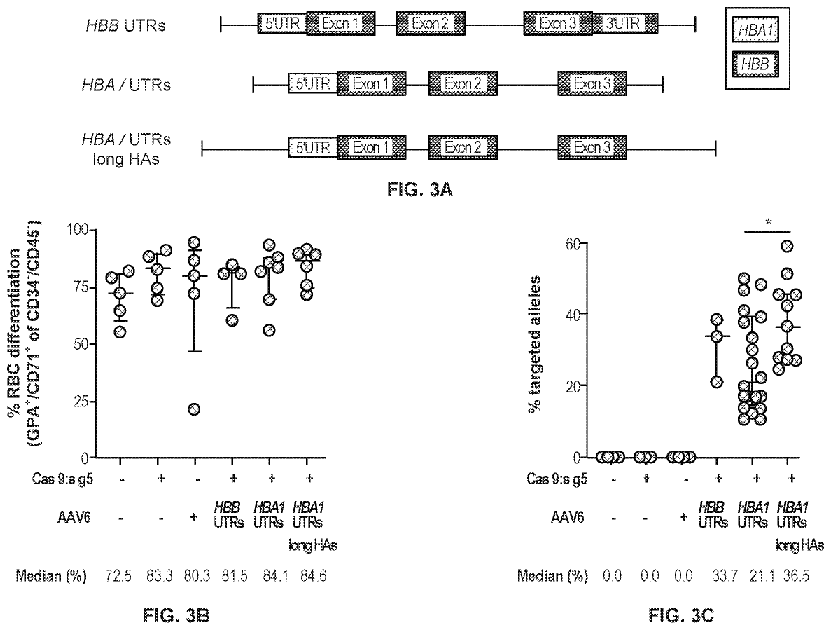

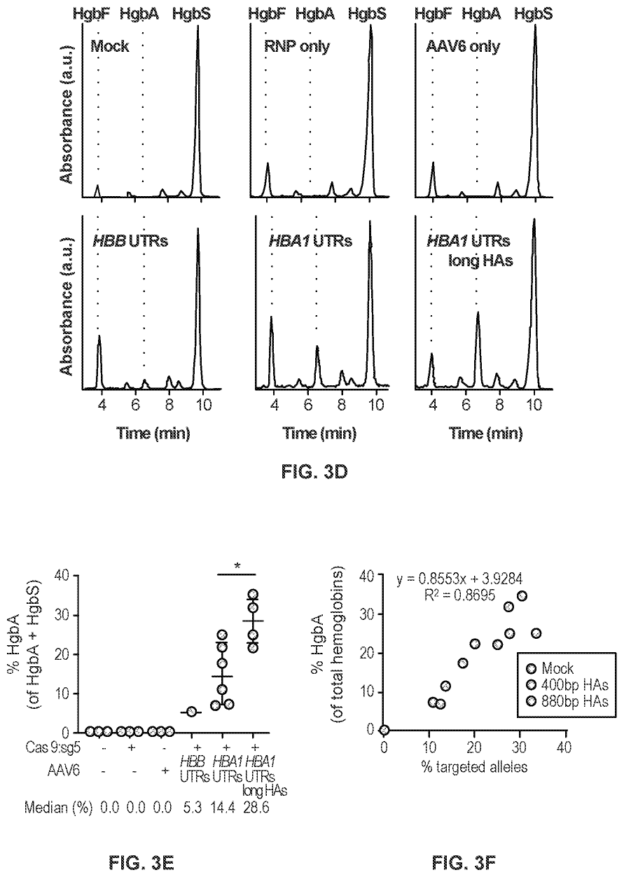

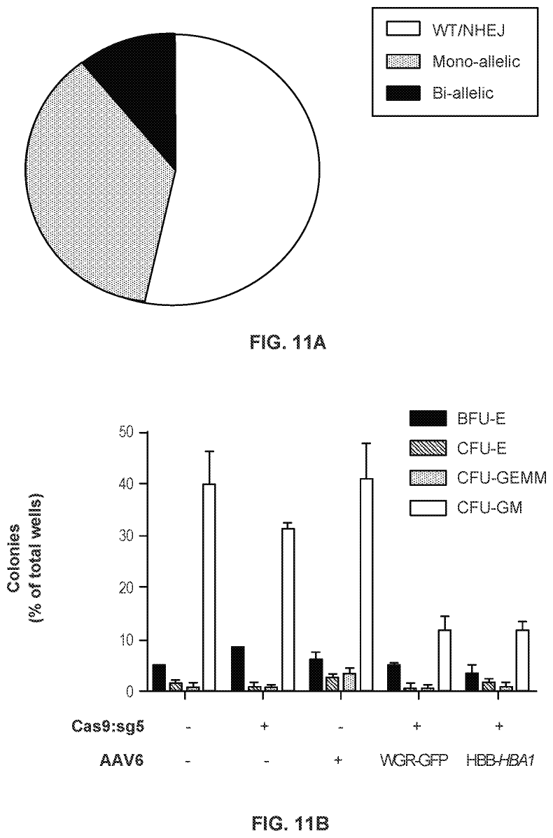

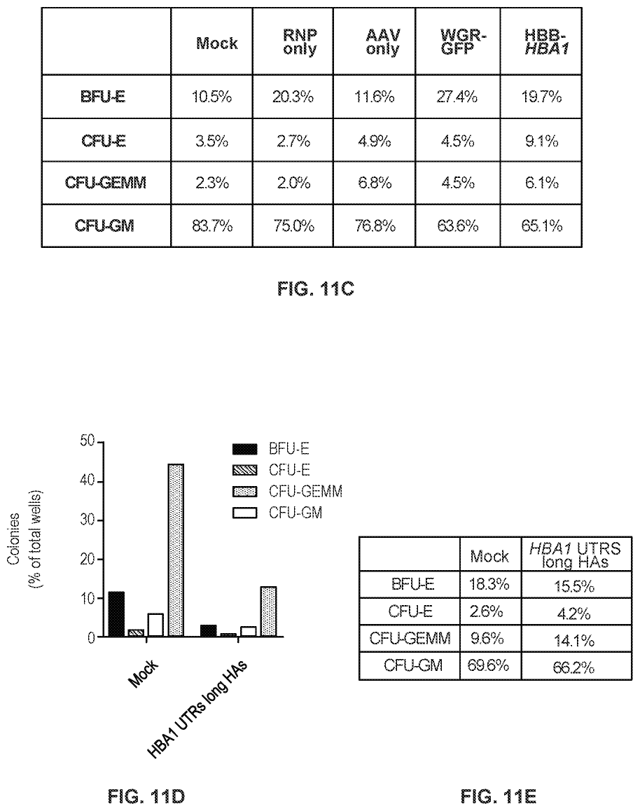

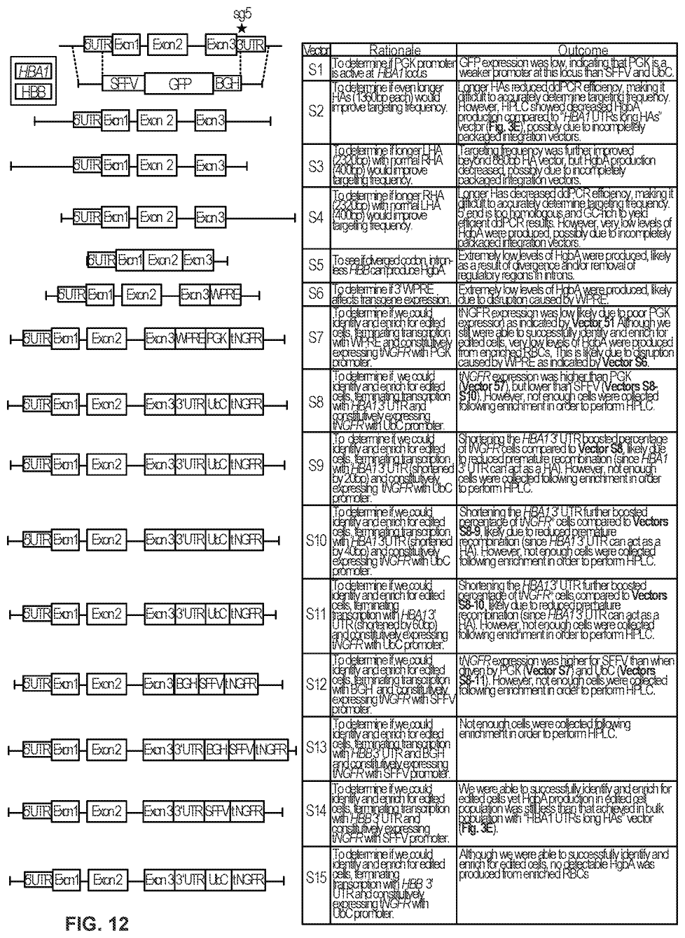

To confirm production of β-globin protein following targeted integration of HBB into HBA1, we screened a number of AAV6 vectors that integrate a HBB transgene alone without a T2A-YFP. These vectors used various combinations of regulatory elements, such as HBB and HBA1 3′ UTRs, WPREs, and BGH poly A regions, as well as a variety of vectors expressing tNGFR that would enable us to identify and enrich for a population of highly edited cells (FIG. 3A; FIG. 11). We also created integration vectors with lengthened left and right homology arms, hypothesizing that doing so could help the cell identify regions of homology—particularly within the left arm that is upstream of the cut site—and thereby increase integration frequency of our HBB transgene. To screen these vectors, we targeted SCD-derived CD34⁺ HSPCs because they exclusively express sickle hemoglobin (HgbS), enabling us to determine degree of adult hemoglobin (HgbA) rescue that results from our editing scheme. As before, targeted HSPCs were edited and then differentiated into RBCs, which indicated that editing at the HBA1 locus had little effect on the ability of cells to differentiate into RBCs (FIG. 3B). We found that integration frequency was significantly improved by lengthening the homology arms, increasing targeted alleles from a mean of 21.1% to 36.5% (P<0.05) (FIG. 3C). From genotyping of single cells plated into 96-well plates, targeting rates of 36.5% of alleles is expected to correspond to 59.5% of cells having undergone at least one editing event (FIG. 11A). When RBCs were analyzed for human hemoglobin by HPLC, we found that all three vectors were able to express and form HgbA tetramers (FIG. 3D). As predicted by the HBB-T2A-YFP vectors, integrations that left local HBA1 UTRs intact yielded a greater amount of HgbA tetramers, indicating that the T2A-YFP system is highly predictive of transgene expression. Interestingly, when HBB-T2A-YFP-edited RBCs were analyzed for hemoglobin tetramer formation by HPLC, we found no HgbA tetramer formation above background (FIG. 12). We believe this may be due to the residual T2A cleavage tail that has been reported to disrupt protein function (37). Nevertheless, we found that the vector with elongated homology arms not only gave rise to significantly higher integration frequency than the vector with 400 bp homology arms, but yielded a significantly greater percentage of HgbA tetramers as well (P<0.05) (FIG. 3E). Importantly, because the vector with the elongated homology arms introduces an identical genome editing event to the HBA1 UTR vector with shorter 400 bp homology arms, we expect this increase in HgbA production to simply be due to the greater frequency at which the long homology arm vector is able to integrate. Consistent with this hypothesis, we found a strong correlation between targeting frequency and HgbA tetramer production (R2=0.8695) (FIG. 3F), indicating that every HBB-targeted HBA1 allele is contributing to endogenous protein levels.

HSPCs Targeted with HBB at HBA1 are Capable of Long-Term Engraftment and Hematopoietic Reconstitution in NSG Mice

To determine whether the editing process impacts the ability of HSPCs to engraft and reconstitute myeloid and lymphoid lineages in vivo, we performed transplantation experiments of human HSPCs targeted at the HBA1 locus into immune-compromised NSG mice. In order to replicate the clinical HSCT process as closely as possible, HSPCs from healthy donors were mobilized using G-CSF and Plerixafor (38). Mobilized peripheral blood was collected and HSPCs were then enriched using the CD34 marker and targeted at HBA1 as above. Two days post-targeting, live CD34⁺ HSPCs were single-cell-sorted into 96-well plates containing methylcellulose media and scored for colony formation ability after incubation for 14d. This indicated that edited HSPCs were able to give rise to cells of all lineages (FIGS. 11B-11C). Although the editing process appears to reduce the total number of colonies, the reduction is primarily due to ability to form colonies in the granulocyte/macrophage lineage, without impacting multi-lineage and erythroid lineage colony formation and their relative distribution.

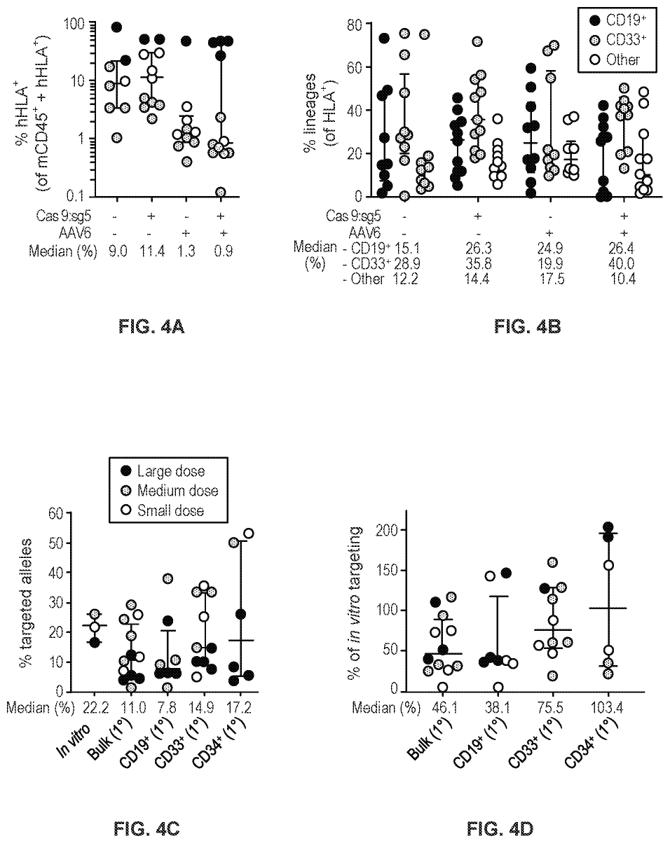

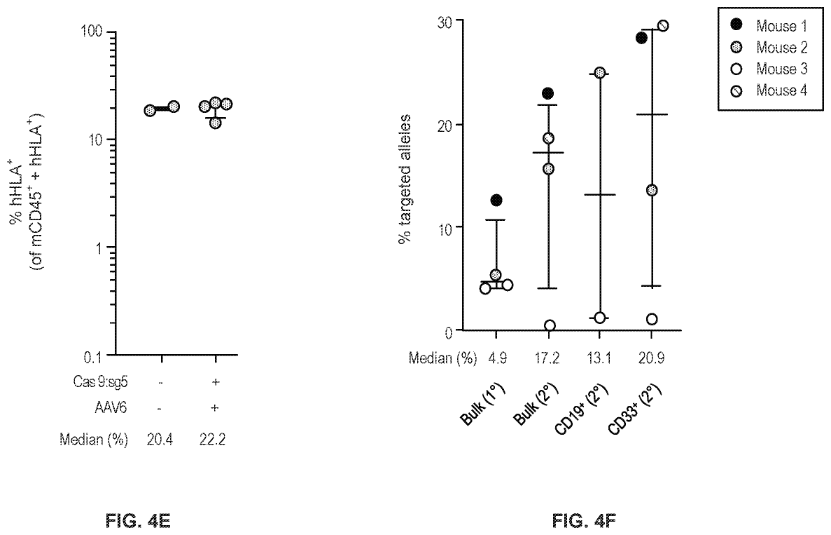

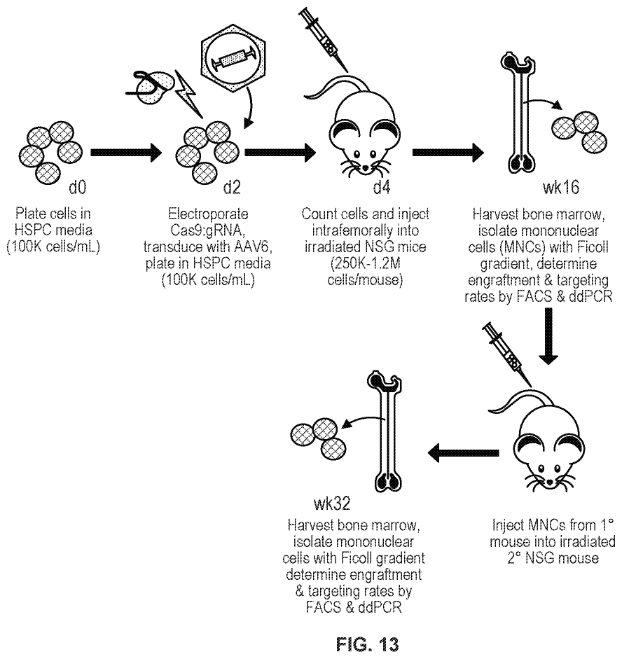

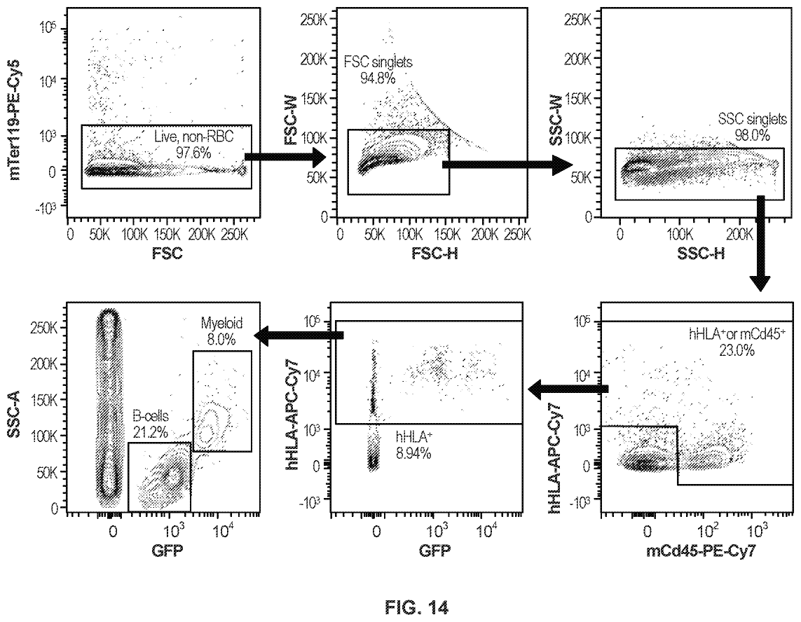

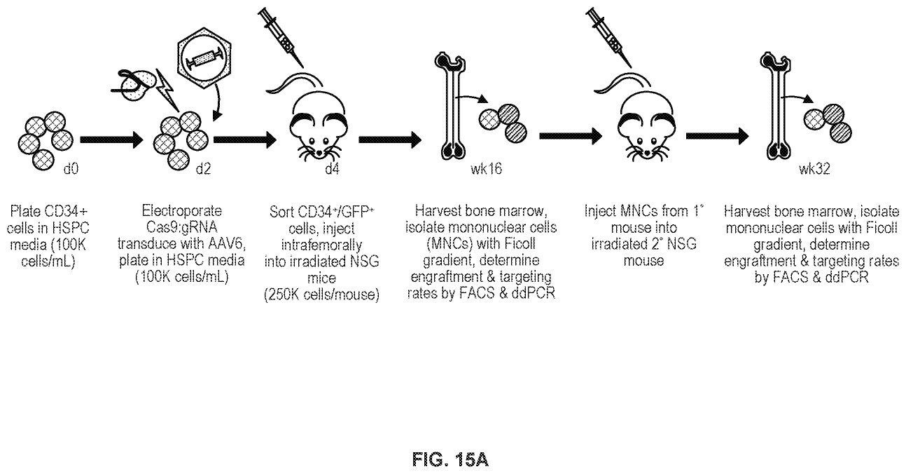

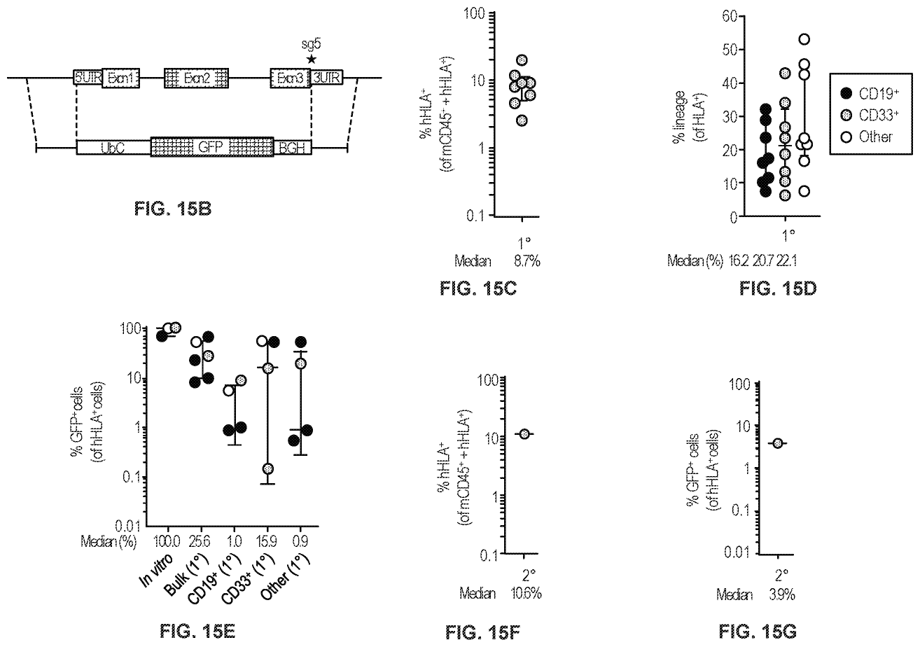

The entire bulk population of edited cells not used toward the colony-forming assay were injected intrafemorally into immunodeficient NSG mice that had been sub-lethally irradiated in order to clear the hematopoietic stem cell niche in the bone marrow (FIG. 13). The experiment was performed on three separate healthy HSPC donors, and due to variable expansion rates among these, the total numbers of cells for transplantation varied among replicates. We therefore designated these as large, medium, and small doses, corresponding to an injection of 1.2 million, 750,000, and 250,000 cells injected per mouse, respectively. Sixteen weeks post-injection, the bone marrow from these mice was harvested and engraftment of human cells was determined using human HLA-A/B/C as a marker (FIG. 14). We found that all three dosages among all treatment groups were able to successfully engraft into the bone marrow (FIG. 4A). We found that among the medium and small doses, engraftment ability of cells was negatively impacted in AAV only and RNP+AAV treatments compared to Mock electroporated and RNP only controls, as observed previously (22, 23). However, when a greater number of cells were transplanted, we no longer observed any significant differences in engraftment among the treatment groups. We also found that the editing process did not affect the ability of human HSPCs to reconstitute myeloid and lymphoid lineages in vivo, and had no discernible impact on the distribution within these lineages among engrafted human cells (FIG. 4B). We next used ddPCR to determine the frequency of the desired targeting event within the population of cells that engrafted. We found that a median of 11.0% of total alleles within our bulk population of successfully-engrafted HSPCs were properly targeted (FIG. 4C), which would be expected to correspond to 17.9% of cells having undergone at least one editing event. We also lineage-sorted engrafted cells into CD19⁺ (B-Cell), CD33⁺ (myeloid), and Lin−/CD10⁻/CD34⁺ (HSPC) populations and determined allele targeting frequencies among these subpopulations, which was a median of 7.8%, 14.9%, and 17.2%, respectively. We observed a modest reduction in targeted allele frequency from the in vitro, pre-transplantation population to the cell population that successfully engrafted (FIG. 4D), which is in line with that observed in previous reports (23, 39) and much less severe of a drop than recently reported by Pattabhi, et al. (40). In addition to editing with the clinically-relevant HBB integration vector, we also targeted cells with a WGR repair template vector to replace the HBA1 gene with a GFP expressed by the strong UbC promoter (FIGS. 15A-15E). We found engraftment rates of human cells at a median of 8.7%, indicating that replacement of the HBA1 gene has little effect on the ability of HSPCs to engraft. We also used flow cytometry to determine editing frequencies of successfully-engrafted cells to be a median of 25.6%, and in B-cell, myeloid, and HSPC lineages at a median of 1.0%, 15.9%, and 0.9%, respectively, indicating that edited cells were capable of engraftment and reconstitution of the various lineages.

After harvesting the cells that successfully engrafted in the initial transplantation experiment, we injected these cells intravenously into new mice as a secondary transplant to determine whether the editing process impacts the ability of cells to engraft and repopulate the hematopoietic system long term in a secondary mouse. Indeed, both the control Mock electroporation treatment as well as cells targeted with RNP/AAV6 were able to engraft at >20% (FIG. 4E). We then used ddPCR as before to determine integration frequency within the population of human cells that were able to successfully engraft in this second round of transplantation. In doing so, we observed integration rates in the bulk sample and within the lineages that were in line with the rates observed among cells that engrafted in the initial transplantation experiment (FIG. 4F). These results were further confirmed by targeting with our WGR GFP vector, which also demonstrated that edited (GFP⁺) cells were able to engraft long term when bone marrow was harvested from secondary mice (FIGS. 15F-15G).

### Delivery of HBB Transgene in β-Thalassemia-Derived HSPCs Corrects α-Globin: β-Globin Imbalance

After demonstrating stable integration frequencies at the HBA1 locus in long-term repopulating HSCs derived from WT donors, we applied the strategy to β-thalassemia-derived HSPCs. CD34⁺ cells were isolated from back-up G-CSF and Plerixafor-mobilized peripheral blood saved from β-thalassemia patients. As previously, we expanded and targeted these HSPCs using the HBA1 UTR vectors (FIG. 3A) and sorted single live CD34⁺ HSPCs into each well of 96-well plates for colony formation assays. We found that edited HSPCs were able to give rise to cells of all lineages (FIGS. 11D-11E). Although no lineage skewing was apparent, the overall ability of edited β-thalassemia-derived HSPCs to form colonies appeared to be slightly reduced, which is in line with prior reports following Cas9/AAV6-mediated genome editing (41).

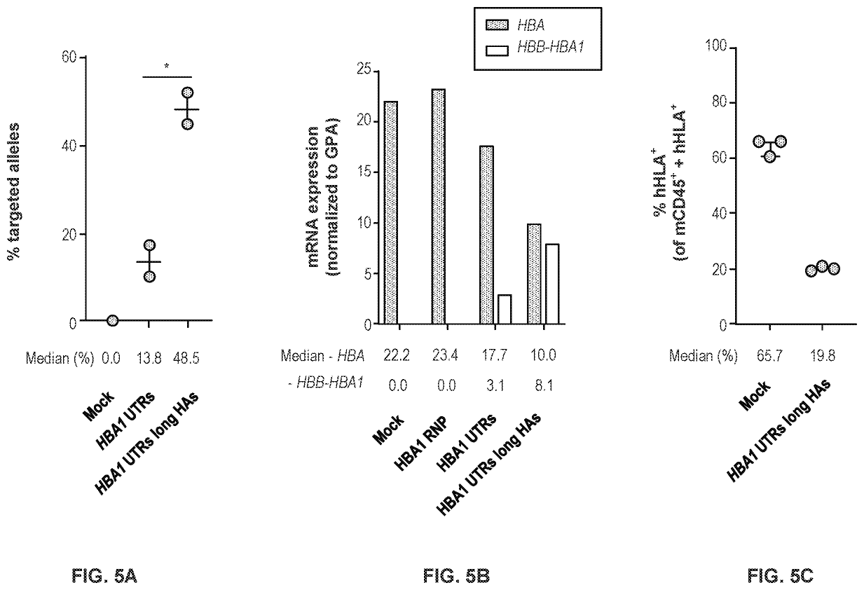

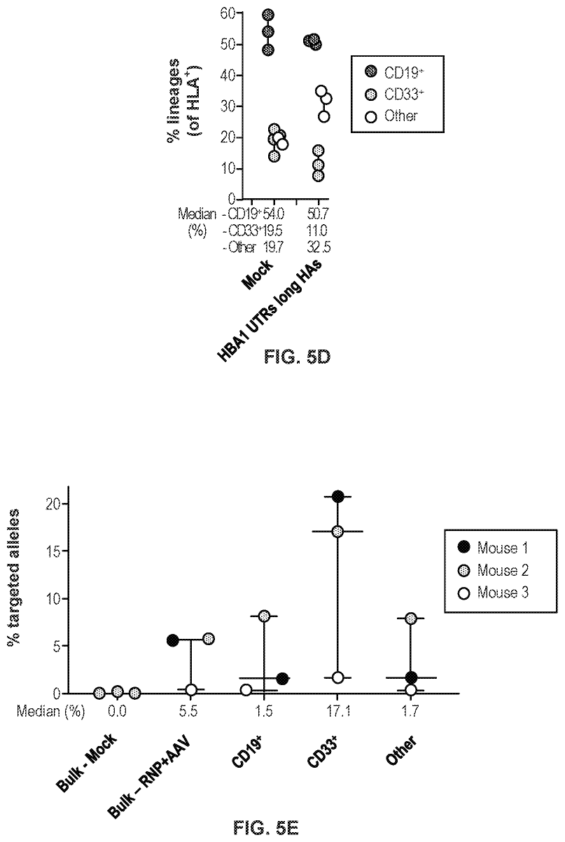

In addition to colony formation assays, a subset of targeted HSPCs underwent RBC differentiation 2 days post-editing. We found that both vectors were able to successfully target these β-thalassemia-derived HSPCs, and, as shown previously, lengthening the homology arms significantly improved editing frequencies in these cells as determined by ddPCR (13.8% vs. 48.5%; P<0.05) (FIG. 5A). To gain insight into how our editing scheme affects expression of α- and β-globin, we designed ddPCR primer/probes that allowed us to assess mRNA expression of α-globin (not distinguishing between HBA1 and HBA2) as well as mRNA expression from the integrated HBB transgene. As anticipated, when expression was normalized to the RBC marker GPA, we found that cells edited with the 400 bp homology arm HBA1 UTRs vector displayed a modest decrease in α-globin expression as well as a modest level of transgene expression (FIG. 5B). Likely due to the higher editing frequency achieved with the elongated homology arm vector, we observed an even greater decrease in α-globin expression as well as an increase in β-globin transgene expression. In fact, we found that the elongated homology arm vector is able to nearly achieve a 1:1 ratio of α-globin:β-globin mRNA expression.

### Targeted β-Thalassemia-Derived HSPCs are Capable of Long-Term Engraftment and Hematopoietic Reconstitution in NSG Mice

In addition to RBC differentiation and analysis, we performed engraftment experiments by injecting targeted β-thalassemia-derived HSPCs into sub-lethally irradiated NSG mice. Sixteen weeks post-transplantation, we harvested bone marrow from mice and determined engraftment and targeting frequencies by flow cytometry and ddPCR, respectively. We found that indeed, patient-derived HSPCs targeted with our transgene at the HBA1 locus were able to successfully engraft, with a median of 19.8% human cells in the bone marrow (FIG. 5C). We also observed that our editing protocol had little bearing on the lineage distribution of cells that successfully engrafted (FIG. 5D). Using ddPCR, we also determined that successfully-engrafted cells were edited in the bulk population at a median frequency of 5.5% as well as in B-cell, myeloid, and HSPC lineages at median frequencies of 1.5%, 17.1%, and 1.7%, respectively (FIG. 5E).

### Discussion

In summary, we have developed a novel genome editing protocol for the potential treatment of β-thalassemia which addresses both molecular factors responsible for the disease—loss of β-globin and accumulation of excess α-globin—in a single genome editing event. Prior data indicates that approximately 25% edited cell chimerism in the bone marrow appears to be the threshold by which transfusion-independence is achieved in thalassemia patients (42). The editing frequencies we have achieved in β-thalassemia-derived HSPCs were 48.5% of alleles targeted in vitro, which is expected to correspond to 79.0% of cells possessing at least one edited allele. Because our approach is site-specific and uses a patient's own cells, it would: 1) Overcome the shortage of immunologically-matched donors; 2) Eliminate the need for constant blood transfusion and/or iron chelation therapy; 3) Eliminate the likelihood of immune rejection that accompanies allogeneic HSCT; and 4) Avoid the risks of semi-random integration of viral vectors in the genome. For these reasons, the technology we have described helps overcome the pitfalls of the current therapeutic strategies.

Beyond the immediate impact for treatment of β-thalassemia, we also believe that our results have broader relevance to the genome editing field as a whole. Prior work had demonstrated that whole gene replacement could occur at low frequencies in T-cells (34), but our work demonstrates that the frequency of whole gene replacement can be significantly increased and harnessed in HSPCs. Because most recessive hereditary diseases are caused by loss-of-function mutations throughout a particular gene, the scheme that we have developed can be adapted into a one-size-fits-all treatment strategy for a wide range of genetic disorders, effectively expanding the genome editing toolbox.

Our study also showed that the T2A cleavage peptide system coupled with a fluorescent reporter was highly predictive of transgene expression. This demonstrates the utility of this system in rapidly identifying successfully-edited cells and for comparison of a variety of integration vectors (i.e., those with different regulatory regions, with or without specific introns, etc.). Because patient-derived HSPCs can be hard to obtain, especially from multiple donors, this T2A screening system also allows for identification of the optimal translational vector in healthy HSPCs that can be validated in patient-derived HSPCs. Lastly, because we have optimized cassette integration at the α-globin locus, a gene only expressed in RBCs, this work has characterized a safe harbor locus for delivery of payloads by RBCs, such as therapeutic enzymes and monoclonal antibodies. This will allow future work to integrate custom vectors at high frequency at the HBA1 locus, thereby achieving RBC-specific expression without the risk of knocking out a gene that is critical to RBC development (because HBA2 remains intact). For these reasons, we expect the findings of this study to guide future genome editing work, both as strategies for correction of a wide range of genetic disorders as well as for a variety of cell engineering applications.

### Methods

### AAV6 Vector Design, Production, and Purification

All AAV6 vectors were cloned into the pAAV-MCS plasmid (Agilent Technologies, Santa Clara, Calif., USA), which contains inverted terminal repeats (ITRs) derived from AAV2. Gibson Assembly Mastermix (New England Biolabs, Ipswich, Mass., USA) was used for the creation of each vector as per manufacturer's instructions. Cut site (CS) vectors were designed such that the left and right homology arms (“LHA” and “RHA”, respectively) are immediately flanking the cut site at either HBA2 or HBA1 gene. Whole gene replacement (WGR) vectors have a LHA flanking the 5′ UTR of either the HBA2 or HBA1 gene while the RHA immediately flanks downstream of its corresponding cut site. The LHA and RHA of every vector is 400 bp, unless otherwise noted, with the vector name (HBA2/HBA1 and CS/WGR) referencing the target integration site and homology arms used, respectively. Within FIG. 1, CS and WGR vectors consisted of a SFFV-GFP-BGH expression cassette. An alternative promoter, UbC, was also used in creating a WGR vector for HBA1 (FIG. 15). In FIG. 2, WGR-T2A-YFP vectors consisted of the full-length HBB gene, unless noted, with a T2A-YFP expression cassette immediately following exon 3 of the HBB gene using the LHA and RHA described previously for WGR. These full-length HBB-T2A-YFP vectors were either flanked by 5′ and 3′ UTRs of HBB, HBA2, or HBA1 as denoted in FIG. 2A. In subsequent experiments, for targeting of SCD or β-thalassemia patient-derived CD34⁺ HSPCs, WGR vectors were designed to target the HBA1 site and contained a full-length HBB gene flanked by either HBA1 UTRs or HBB UTRs. While the HBB UTRs' and HBA1 UTRs' vector both share 400 bp HAs, the HBA1 UTRs long HAs' vector was modified to have 880 bp HAs. Few modifications were made to the production of AAV6 vectors as described (43). 293T cells (Life Technologies, Carlsbad, Calif., USA) were seeded in ten 15 cm² dishes with 13-15×10⁶ cells per plate. 24 h later, each dish was transfected with a standard polyethylenimine (PEI) transfection of 6 μg ITR-containing plasmid and 22 μg pDGM6, which contains the AAV6 cap genes, AAV2 rep genes, and Ad5 helper genes. After a 48-72 h incubation, cells were lysed by 3 freeze-thaw cycles, treated with benzonase (Thermo Fisher Scientific, Waltham, Mass., USA) at 250 U/mL, and the vector was then purified through an iodixanol gradient centrifugation at 48,000 RPM for 2.25 h at 18° C. Afterwards, full capsids were isolated at the 40-58% iodixanol interface and then stored at 80° C. until further use. As an alternative method, AAVPro Purification Kit (All Serotypes) (Takara Bio USA, Mountain View, Calif., USA) were also used following the 48-72 h incubation period, to extract full AAV6 capsids as per manufacturer's instructions. AAV6 vectors were titered using ddPCR to measure number of vector genomes as previously described (44).

### Culturing of CD34⁺ HSPCs

Human CD34⁺ HSPCs were cultured as previously described (19, 23, 33, 41, 45, 46). CD34⁺ HSPCs were sourced from fresh cord blood, frozen cord blood and Plerixafor- and/or G-CSF-mobilized peripheral blood (AllCells, Alameda, Calif., USA and STEMCELL Technologies, Vancouver, Canada), frozen Plerixafor- and/or G-CSF-mobilized peripheral blood of patients with SCD, and frozen G-CSF and Plerixafor-mobilized peripheral blood from β-thalassemia patients. CD34⁺ HSPCs were cultured at 2.5×10⁵-5×10⁵ cells/mL in StemSpan SFEM II (STEMCELL Technologies, Vancouver, Canada) base medium supplemented with stem cell factor (SCF) (100 ng/mL), thrombopoietin (TPO) (100 ng/mL), FLT3—ligand (100 ng/mL), IL-6 (100 ng/mL), UM171 (35 nM), 20 mg/mL streptomycin, and 20 U/mL penicillin. The cell incubator conditions were 37° C., 5% CO₂, and 5% O₂.

### Genome Editing of CD34⁺ HSPCs

Chemically-modified sgRNAs used to edit CD34⁺ HSPCs at either HBA2 or HBA1 were purchased from Synthego (Menlo Park, Calif., USA) and TriLink BioTechnologies (San Diego, Calif., USA) and were purified by high-performance liquid chromatography (HPLC). The sgRNA modifications added were the 2′-O-methyl-3′-phosphorothioate at the three terminal nucleotides of the 5′ and 3′ ends described previously (29). The target sequences for sgRNAs were as follows: sg1: 5′-CTACCGAGGCTCCAGCTTAA-3′ (SEQ ID NO: 1); sg2: 5′-GGCAGGAGGAACGGCTACCG-3′ (SEQ ID NO: 2); sg3: 5′-GGGGAGGAGGGCCCGTTGGG-3′ (SEQ ID NO: 3); sg4: 5′-CCACCGAGGCTCCAGCTTAA-3′ (SEQ ID NO: 4); and sg5: 5′-GGCAAGAAGCATGGCCACCG-3′ (SEQ ID NO: 5). All Cas9 protein (Alt-R S.p. Cas9 Nuclease V3) used was purchased from Integrated DNA Technologies (Coralville, Iowa, USA). The RNPs were complexed at a Cas9:sgRNA molar ratio of 1:2.5 at 25° C. for 10 min prior to electroporation. CD34⁺ cells were resuspended in P3 buffer (Lonza, Basel, Switzerland) with complexed RNPs and electroporated using the Lonza 4D Nucleofector (program DZ-100). Cells were plated at 2.5×10⁵ cells/mL following electroporation in the cytokine-supplemented media described previously. Immediately following electroporation, AAV6 was supplied to the cells at 5×10³-1×10⁴ vector genomes/cell based on titers determined by ddPCR.

### Indel Frequency Analysis by TIDE

2-4 d post-targeting, HSPCs were harvested and QuickExtract DNA extraction solution (Epicentre, Madison, Wis., USA) was used to collect gDNA. The following primers were then used to amplify respective cut sites at HBA2 and HBA1 along with CleanAmp PCR 2× Master Mix (TriLink, San Diego, Calif., USA) according to manufacturer's instructions: HBA2 (sg1-3): forward: 5′-CCCGAAAGGAAAGGGTGGCG-3′ (SEQ ID NO: 9) reverse: 5′-TGGCACCTGCACTTGCACTG-3′ (SEQ ID NO: 10); HBA1 (sg4-5): forward: 5′-TCCGGGGTGCACGAGCCGAC-3′ (SEQ ID NO: 11), reverse: 5′-GCGGTGGCTCCACTTTCCCT-3′ (SEQ ID NO: 12). PCR reactions were then run on a 1% agarose gel and appropriate bands were cut and gel-extracted using a GeneJET Gel Extraction Kit (Thermo Fisher Scientific, Waltham, Mass., USA) according to manufacturer's instructions. Gel-extracted amplicons were then Sanger sequenced with the following primers: HBA2 (sg1-3): forward: 5′-GGGGTGCGGGCTGACTTTCT-3′ (SEQ ID NO: 13) reverse: 5′-CTGAGACAGGTAAACACCTCCAT-3′ (SEQ ID NO: 14); HBA1 (sg4-5): forward: 5′-TGGAGACGTCCTGGCCCC-3′ (SEQ ID NO: 15), reverse: 5′-CCTGGCACGTTTGCTGAGG-3′ (SEQ ID NO: 16). Resulting Sanger chromatograms were the used as input for indel frequency analysis by TIDE as previously described (30).

### Gene Targeting Analysis by Flow Cytometry

4-8 d post-targeting with fluorescent integration vectors, CD34⁺ HSPCs were harvested and the percentage of edited cells was determined by flow cytometry. Cells were analyzed for viability using Ghost Dye Red 780 (Tonbo Biosciences, San Diego, Calif., USA) and reporter expression was assessed using either the Accuri C6 flow cytometer (BD Biosciences, San Jose, Calif., USA) or FACS Aria II (BD Biosciences, San Jose, Calif., USA). The data was subsequently analyzed using FlowJo (FlowJo LLC, Ashland, Oreg., USA).

Allelic Targeting Analysis by ddPCR

2-4 d post-targeting, HSPCs were harvested and QuickExtract DNA extraction solution (Epicentre, Madison, Wis., USA) was used to collect gDNA. gDNA was then digested using BAMH1-HF as per manufacturer's instructions (New England Biolabs, Ipswich, Mass., USA). The percentage of targeted alleles within a cell population was measured by ddPCR using the following reaction mixture: 1-4 μL of digested gDNA input, 10 μL ddPCR SuperMix for Probes (No dUTP) (Bio-Rad, Hercules, Calif., USA), primer/probes (1:3.6 ratio; Integrated DNA Technologies, Coralville, Iowa, USA), volume up to 20 μL with H₂O. ddPCR droplets were then generated following the manufacturer's instructions (Bio-Rad, Hercules, Calif., USA): 20 μL of ddPCR reaction, 70 μL droplet generation oil, and 40 μL of droplet sample. Thermocycler (Bio-Rad, Hercules, Calif., USA) settings were as follows: 1. 98° C. (10 min), 2. 94° C. (30 s), 3. 57.3° C. (30 s), 4. 72° C. (1.75 min) (return to step 2×40-50 cycles), 5. 98° C. (10 min). Analysis of droplet samples was done using the QX200 Droplet Digital PCR System (Bio-Rad, Hercules, Calif., USA). To determine percentage of alleles targeted, the number of Poisson-corrected integrant copies/mL were divided by the number of Poisson-corrected reference DNA copies/mL. The following primers and 6-FAM/ZEN/IBFQ-labelled hydrolysis probes were purchased as custom-designed PrimeTime qPCR Assays from Integrated DNA Technologies (Coralvilla, Iowa, USA): All HBA2-integrating GFP vectors (spans from BGH to outside 400 bp HBA2 RHA): forward: 5′-TAGTTGCCAGCCATCTGTTG-3′ (SEQ ID NO: 17), reverse: 5′-GGGGACAGCCTATTTTGCTA-3′ (SEQ ID NO: 18), probe: 5′-AAATGAGGAAATTGCATCGC-3′ (SEQ ID NO: 19); All HBA1-integrating GFP vectors (spans from BGH to outside 400 bp HBA1 RHA): forward: 5′-TAGTTGCCAGCCATCTGTTG-3′ (SEQ ID NO: 17), reverse: 5′-TAGTGGGAACGATGGGGGAT-3′ (SEQ ID NO: 20), probe: 5′-AAATGAGGAAATTGCATCGC-3′ (SEQ ID NO: 19); HBA2-integrating HBB-T2A-YFP vector (spans from YFP to outside 400 bp HBA2 RHA): forward: 5′-AGTCCAAGCTGAGCAAAGA-3′ (SEQ ID NO: 21), reverse: 5′-GGGGACAGCCTATTTTGCTA-3′ (SEQ ID NO: 18), probe: 5′-CGAGAAGCGCGATCACATGGTCCTGC-3′ (SEQ ID NO: 22); All HBA1-integrating HBB-T2A-YFP vectors (spans from YFP to outside 400 bp HBA1 RHA): forward: 5′-AGTCCAAGCTGAGCAAAGA-3′ (SEQ ID NO: 21), reverse: 5′-TAGTGGGAACGATGGGGGAT-3′ (SEQ ID NO: 20), probe: 5′-CGAGAAGCGCGATCACATGGTCCTGC-3′ (SEQ ID NO: 22); HBA1-integrating HBB vectors (with 400 bp HAs, without T2A-YFP) (spans from HBB exon 3 to outside 400 bp HBA1 RHA): forward: 5′-GCTGCCTATCAGAAAGTGGT-3′ (SEQ ID NO: 23), reverse: 5′-TAGTGGGAACGATGGGGGAT-3′ (SEQ ID NO: 20), probe: 5′-CTGGTGTGGCTAATGCCCTGGCCC-3′ (SEQ ID NO: 24); HBA1-integrating HBB vector (with 880 bp HAs, without T2A-YFP) (spans from HBB exon 3 to outside 880 bp HBA1 RHA): forward: 5′-GCTGCCTATCAGAAAGTGGT-3′ (SEQ ID NO: 23), reverse: 5′-ATCACAAACGCAGGCAGAG-3′ (SEQ ID NO: 25), probe: 5′-CTGGTGTGGCTAATGCCCTGGCCC-3′ (SEQ ID NO: 24). The primers and HEX/ZEN/IBFQ-labelled hydrolysis probe purchased as custom-designed PrimeTime qPCR Assays from Integrated DNA Technologies (Coralvilla, Iowa, USA) were used to amplify the CCRL2 reference gene: forward: 5′-GCTGTATGAATCCAGGTCC-3′ (SEQ ID NO: 26), reverse: 5′-CCTCCTGGCTGAGAAAAAG-3′ (SEQ ID NO: 27), probe: 5′-TGTTTCCTCCAGGATAAGGCAGCTGT-3′ (SEQ ID NO: 28). Due to the length of the ‘HBA1 UTRs long HAs’ vector and to ensure episomal AAV is not detected, the ddPCR amplicon exceeds the template size recommended by the ddPCR manufacturer. Upon analysis of the data, the percentage of targeted alleles of this vector is underestimated. Therefore, in these instances a correction factor to account for this underestimation was determined by amplifying gDNA harvested from HSPCs targeted with HBA1 UTRs vector with 400 bp HAs with both sets of ddPCR primers and probes (those for vectors with 400 bp and 880 bp HAs) as well as CCRL2 reference probes. The resulting correction factor was then applied to the targeted allele percentage from samples targeted with and amplified with primers and probe for 880 bp HAs.

Off-Target Activity Analysis by rhAmpSeq

Predicted off-target sites for HBA1 sg5 were identified using COSMID with up to three mismatches allowed in the 19 PAM-proximal bases and the PAM sequence NGG. Multiplexed PCR amplicon sequencing to calculate total editing frequencies for the 40 most highly-predicted off-target sites was performed using rhAmpSeq technology (Integrated DNA Technologies). NGS data were analyzed using a custom-built pipeline. PCR amplicons were sequenced on an Illumina MiSeq (v2 chemistry; 2×150) and data demultiplexed using Picard tools v2.9 (https://github.com/broadinstitute/picard). Forward and reverse reads were merged into extended amplicons (flash v1.2.11) (47) before being aligned against the GRCh38 genomic reference (minimap2 v2.12) (48). Reads were assigned to targets in the multiplex primer pool (bedtools tags v2.25) (49) and re-aligned to the target, favoring alignment choices with indels near the predicted Cas9 cut site. At each target, editing was calculated as the percentage of total reads containing an indel within a 4 bp window of the cut site.

In Vitro Differentiation of CD34⁺ HSPCs into Erythrocytes

Following targeting, HSPCs derived from healthy, SCD, or β-thalassemia patients were cultured for 14-16 d at 37° C. and 5% CO₂ in SFEM II medium (STEMCELL Technologies, Vancouver, Canada) as previously described (35, 36). SFEMII base medium was supplemented with 100 U/mL penicillin-streptomycin, 10 ng/mL SCF, 1 ng/mL IL-3 (PeproTech, Rocky Hill, N.J., USA), 3 U/mL erythropoietin (eBiosciences, San Diego, Calif., USA), 200 μg/mL transferrin (Sigma-Aldrich, St. Louis, Mo., USA), 3% antibody serum (heat-inactivated from Atlanta Biologicals, Flowery Branch, Ga., USA), 2% human plasma (umbilical cord blood), 10 μg/mL insulin (Sigma-Aldrich, St. Louis, Mo., USA) and 3 U/mL heparin (Sigma-Aldrich, St. Louis, Mo., USA). In the first phase, d 0-7 (day zero being 2d post-targeting) of differentiation, cells were cultured at 1×10⁵ cells/mL. In the second phase, d7-10, cells were maintained at 1×10⁵ cells/mL, and IL-3 was removed from the culture. In the third phase, d11-16, cells were cultured at 1×10⁶ cells/mL, and transferrin was increased to 1 mg/mL within the culture medium.

mRNA Analysis

Following differentiation of HSPCs into erythrocytes, cells were harvested and RNA was extracted using RNeasy Plus Mini Kit (Qiagen, Hilden, Germany). Subsequently, cDNA was made from approximately 100 ng RNA using the iScript Reverse Transcription Supermix for RT-qPCR (Bio-Rad, Hercules, Calif., USA). Expression levels of β-globin transgene and α-globin mRNA were quantified by ddPCR using the following primers and 6-FAM/ZEN/IBFQ-labelled hydrolysis probes purchased as custom-designed PrimeTime qPCR Assays from Integrated DNA Technologies (Coralvilla, Iowa, USA): HBB: forward: 5′-GAGAACTTCAGGCTCCTG-3′ (SEQ ID NO: 29), reverse: 5′-CGGGGGTACGGGTGCAGGAA-3′ (SEQ ID NO: 30), probe: 5′-TGGCCATGCTTCTTGCCCCT-3′ (SEQ ID NO: 31); HBA (does not distinguish between HBA2 and HBA1): forward: 5′-GACCTGCACGCGCACAAGCTT-3′ (SEQ ID NO: 32), reverse: 5′-GCTCACAGAAGCCAGGAACTTG-3′ (SEQ ID NO: 33), probe: 5′-CAACTTCAAGCTCCTAAGCCA-3′ (SEQ ID NO: 34). To normalize for RNA input, levels of the RBC-specific reference gene GPA was determined in each sample using the following primers and HEX/ZEN/IBFQ-labelled hydrolysis probes purchased as custom-designed PrimeTime qPCR Assays from Integrated DNA Technologies (Coralvilla, Iowa, USA): forward: 5′-ATATGCAGCCACTCCTAGAGCTC-3′ (SEQ ID NO: 35), reverse: 5′-CTGGTTCAGAGAAATGATGGGCA-3′ (SEQ ID NO: 36), probe: 5′-AGGAAACCGGAGAAAGGGTA-3′ (SEQ ID NO: 37). ddPCR reactions were created using the respective primers and probes and droplets were generated as described above. Thermocycler (Bio-Rad, Hercules, Calif., USA) settings were as follows: 1. 98° C. (10 min), 2. 94° C. (30 s), 3. 59.4° C. (30 s), 4. 72° C. (30 s) (return to step 2×40-50 cycles), 5. 98° C. (10 min). Analysis of droplet samples was done using the QX200 Droplet Digital PCR System (Bio-Rad, Hercules, Calif., USA). To determine relative expression levels, the number of Poisson-corrected HBA or HBB transgene copies/mL were divided by the number of Poisson-corrected GPA copies/mL.

### Immunophenotyping of Differentiated Erythrocytes

HSPCs subjected to the above erythrocyte differentiation were analyzed at d14-16 for erythrocyte lineage-specific markers using a FACS Aria II (BD Biosciences, San Jose, Calif., USA). Edited and non-edited cells were analyzed by flow cytometry using the following antibodies: hCD45 V450 (HI30; BD Biosciences, San Jose, Calif., USA), hCD34 APC (561; BioLegend, San Diego, Calif., USA), hCD71 PE-Cy7 (OKT9; Affymetrix, Santa Clara, Calif., USA), and hCD235a PE (GPA) (GA-R2; BD Biosciences, San Jose, Calif., USA).

### Steady-State Hemoglobin Tetramer Analysis

HSPCs subjected to the above erythrocyte differentiation were lysed using water equivalent to three volumes of pelleted cells. The mixture was incubated at room temperature for 15 min, followed by 30 s sonication. For separation of lysate from the erythrocyte ghosts, centrifugation was performed at 13,000 RPM for 5 min. HPLC analysis of hemoglobins in their native form were analyzed on a weak cation-exchange PolyCAT A column (100×4.6-mm, 3 μm, 1,000 Å) (PolyLC Inc., Columbia, Md., USA) using a Shimadzu UFLC system at room temperature. Mobile phase A (MPA) consists of 20 mM Bis-tris+2 mM KCN, pH 6.96. Mobile phase B (MPB) consists of 20 mM Bis-tris+2 mM KCN+200 mM NaCl, pH 6.55. Clear hemolysate was diluted four times in MPA, and then 20 μL was injected onto the column. A flow rate of 1.5 mL/min and the following gradients were used in time (min)/% B organic solvent: (0/10%; 8/40%; 17/90%; 20/10%; 30/stop).

### Methylcellulose CFU Assessment

2d post-targeting, HSPCs were stained using CD34 APC (561; BioLegend, San Diego, Calif., USA), Ghost Dye Red 780 Viability Dye (Tonbo Biosciences, San Diego, Calif., USA) and live CD34⁺ cells were sorted into 96-well plates containing MethoCult Optimum (STEMCELL Technologies, Vancouver, Canada). After 12-16 d, colonies were appropriately scored based on external appearance in a blinded fashion.

CD34′ HSPC Transplantation into Immunodeficient NSG Mice

Six- to eight-week-old all-female NSG mice (Jackson Laboratory, Bar Harbor, Me., USA) were irradiated using 200 rads of radiation 12-24 h prior to transplantation with targeted HSPCs (2d post-targeting) via intrafemoral or tail-vein injections. Approximately 2.5×10⁵-1.3×10⁶ electroporated HSPCs (exact number noted in figures) were injected using an insulin syringe with a 27G, 0.5 inch (12.7 mm) needle. This experimental protocol was approved by Stanford University's Administrative Panel on Laboratory Animal Care. No blinding to group allocation, treatment randomization, or exclusion criteria during data collection or analysis was necessary in order to account for unintended bias in any experiment reported in the manuscript. All reported experiments were completed in compliance with the institutional Animal Care and Use Committee (IACUC; protocol number D16-00134) administered at Stanford by the Administrative Panel on Laboratory Animal Care (APLAC; protocol number 25065) in accordance with Stanford University policy. Sample sizes used in this study were within the range reported in previous Cas9/AAV6-mediated genome editing studies (21-23).

### Assessment of Human Engraftment

15-17 wks post-transplantation of CD34⁺-edited HSPCs, mice were euthanized and bone marrow was harvested from tibia, femurs, pelvis, sternum, and spine using a pestle and mortar. Mononuclear cells were enriched using a Ficoll gradient centrifugation (Ficoll-Paque Plus, GE Healthcare, Chicago, Ill.) for 25 min at 2,000 g at room temperature. The samples were then stained for 30 min at 4° C. with the following antibodies: monoclonal hCD33 V450 (WM53; BD Biosciences, San Jose, Calif., USA); hHLA-A/B/C FITC (W6/32; BioLegend, San Diego, Calif., USA); CD19 PerCp-Cy5.5 (HIB19; BD Biosciences); mTer119 PE-Cy5 (TER-119; eBiosciences, San Diego, Calif., USA); mCd45.1 PE-Cy7 (A20; eBiosciences, San Diego, Calif., USA); hGPA PE (HIR2; eBiosciences, San Diego, Calif., USA); hCD34 APC (581; BioLegend, San Diego, Calif., USA); and hCD10 APC-Cy7 (HI10a; BioLegend, San Diego, Calif., USA). Multi-lineage engraftment was established by the presence of myeloid cells (CD33⁺) and B-cells (CD19⁺) of engrafted human cells (Cd45⁺; HLA-A/B/C⁺ cells). For GFP-expressing cells, hHLA-A/B/C-APC-Cy7 (W6/32; BioLegend, San Diego, Calif., USA) was used rather than hHLA-FITC. For secondary transplantation, only a portion of the primary mouse mononuclear population was stained, and the rest (2.5×10⁵ cells-1.3×10⁶ cells) were transplanted into six- to eight-week-old NSG mice post-irradiation conditioning. Cells were the assessed in same aforementioned manner 16 wks post-transplantation into secondary mice.

### Statistical Analysis

All data points presented in the figures were taken from distinct treatment groups rather than repeated measurements of the same treatment. Sample sizes used in this study were within the range reported in previous Cas9/AAV6-mediated genome editing studies (21-23). No data exclusion criteria were established prior to the execution of any experiments reported in this paper, and no data were excluded following conclusion of the experiments. Where possible, all experiments were replicated across a minimum of three or more CD34⁺ HSPC donors. The one exception to this is the data reported in FIG. 5 that is derived from a single HSPC donor, which is due to our limited access to β-thalassemia patients. All statistical tests on experimental groups were done using Prism7 GraphPad Software. Two-tailed unpaired t tests were used to determine statistical differences among treatment groups. Sample variance was determined for all treatment groups, and where found to be unequal, Welch's t test also confirmed statistical significance.

### Example 2. Additional Experiments and Data

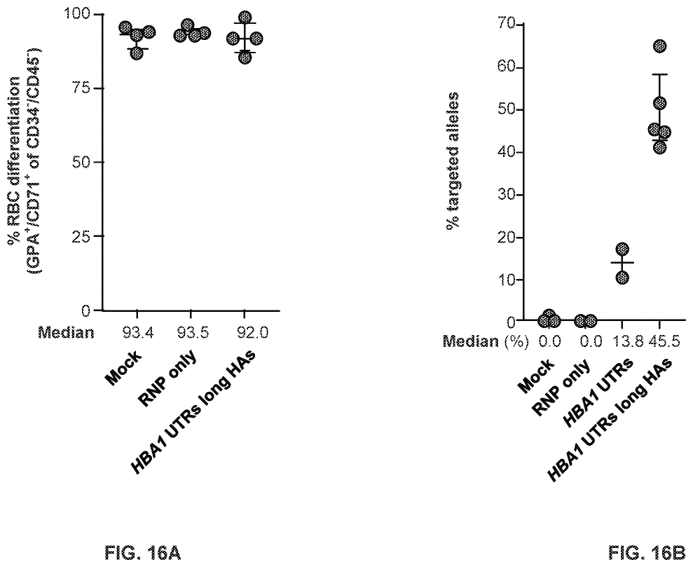

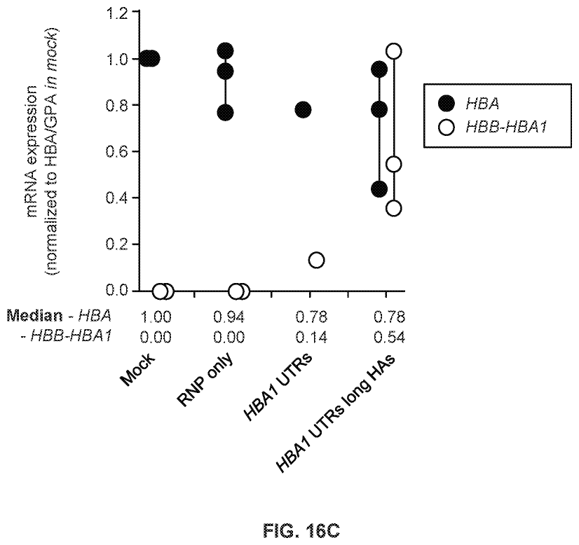

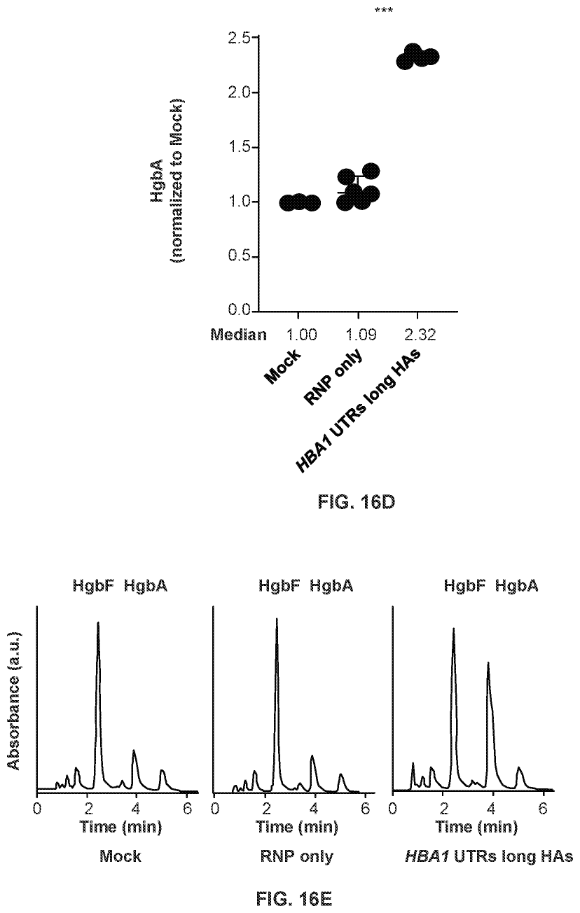

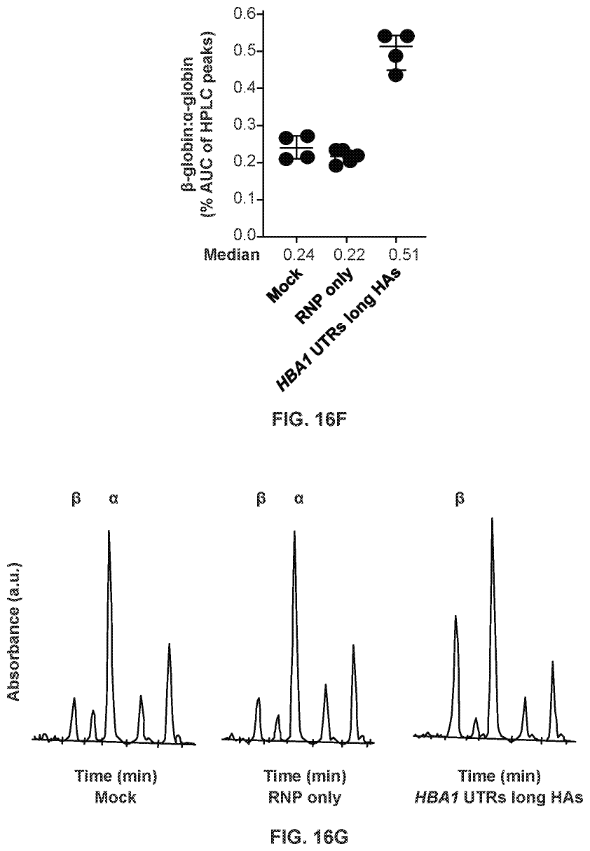

We examined the targeting, β-globin production, and engraftment data in β-thalassemia patient-derived HSPCs. FIG. 16A shows the percentage of CD34⁻/CD45⁻ HSPCs that acquire RBC surface markers, GPA and CD71, as determined by flow cytometry, and FIG. 16B shows the targeted allele frequency at HBA1 in β-thalassemia-derived HSPCs as determined by ddPCR. Following differentiation of targeted HSPCs into RBCs, mRNA was harvested and converted into cDNA, and the expression of HBA (which does not distinguish between HBA1 and HBA2) and HBB transgene were normalized to HBG expression (FIG. 16C). Hemoglobin tetramer HPLC results showing HgbA normalized to HgbF are shown in FIG. 16D, and representative hemoglobin tetramer HPLC plots for each treatment following targeting and RBC differentiation of HSPCs are shown in FIG. 16E. Retention time for HgbF and HgbA tetramer peaks are indicated. FIG. 16F provides a summary of reverse-phase globin chain HPLC results showing area under the curve (AUC) of β-globin/AUC of α-globin, and FIG. 16G presents representative reverse-phase globin chain HPLC plots for each treatment following targeting and RBC differentiation of HSPCs.

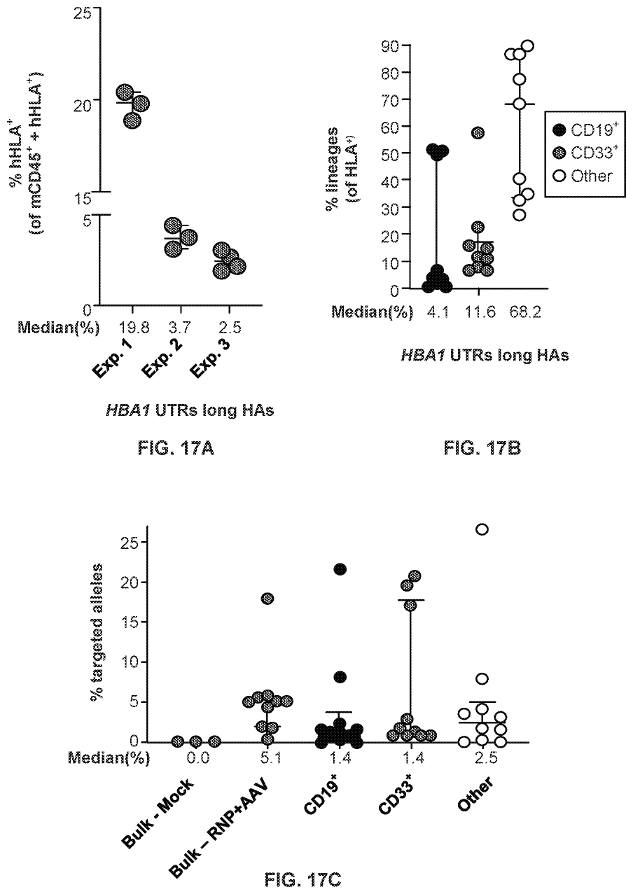

16 weeks after bone marrow transplantation of targeted β-thalassemia-derived HSPCs into NSG mice, bone marrow was harvested and rates of engraftment were determined (FIG. 17A). Among engrafted human cells, the distribution among B-cell, myeloid, or other (i.e., HSPC/RBC/T/NK/Pre-B) lineages are shown in FIG. 17B. The targeted allele frequency at HBA1 is shown in FIG. 17C, as determined by ddPCR among engrafted human cells in bulk sample as well as among CD19⁺ (B-cell), CD33⁺ (myeloid), and other (i.e., HSPC/RBC/T/NK/Pre-B) lineages in secondary transplantation experiments.

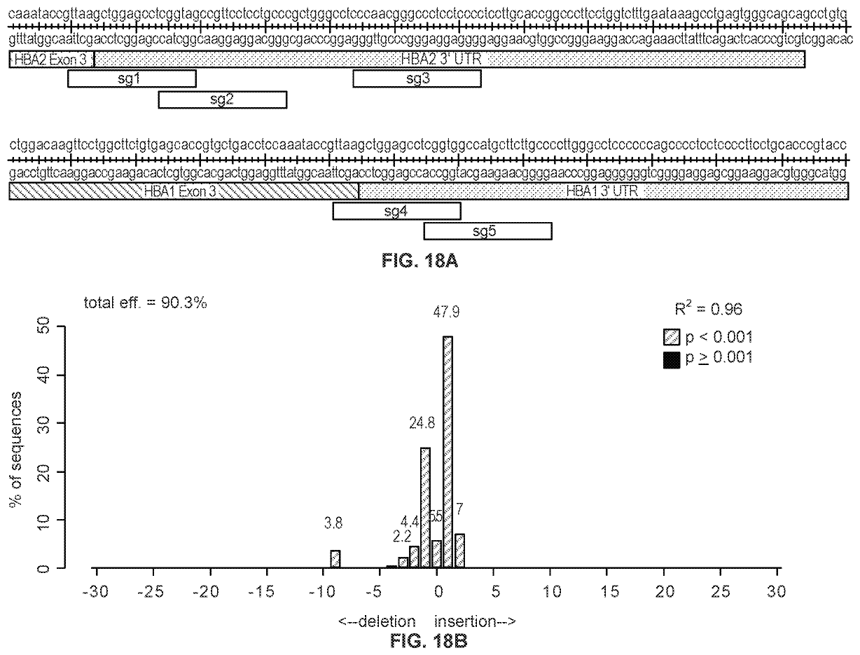

We further examined additional aspects of the indel spectrum generated by the HBA1-targeting gRNA 5. FIG. 18A provides a schematic depicting locations of all five guide sequences at genomic loci, and FIG. 18B presents a representative indel spectrum of HBA1-specific sg5 generated by TIDE software.

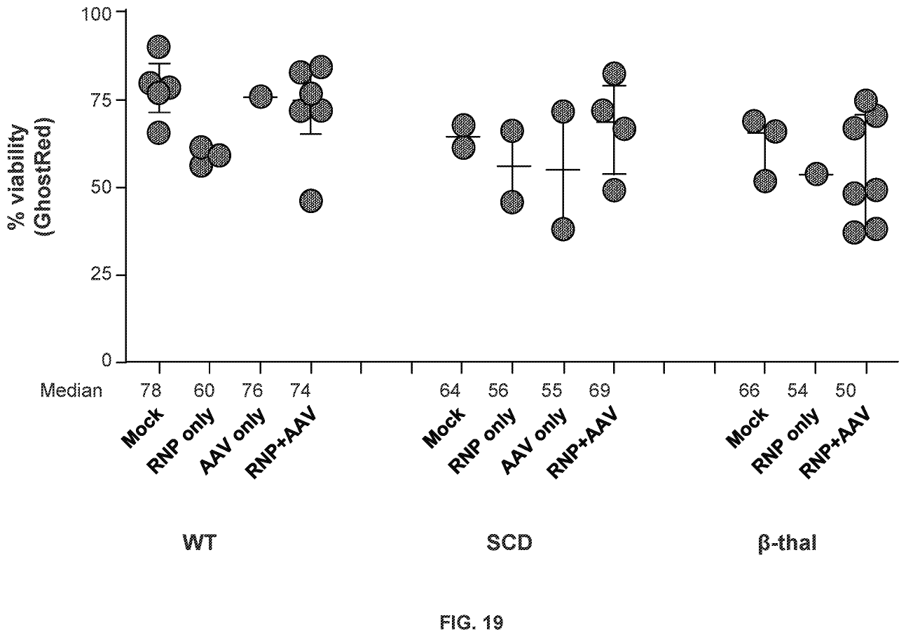

We also examined viability data post-targeting in HSPCs. HSPC viability was quantified 2-4d post-editing by flow cytometry, and the percentage of cells that stained negative for GhostRed viability dye determined (FIG. 19). All cells were edited with our optimized HBB gene replacement vector using standard conditions (i.e., electroporation of Cas9 RNP+sg5, 5K MOI of AAV, and no AAV wash at 24 h).

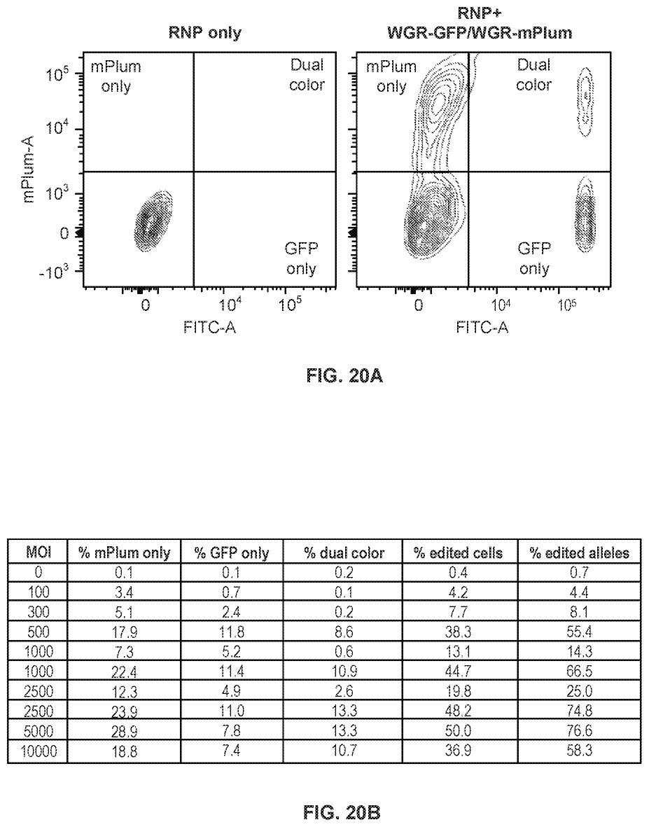

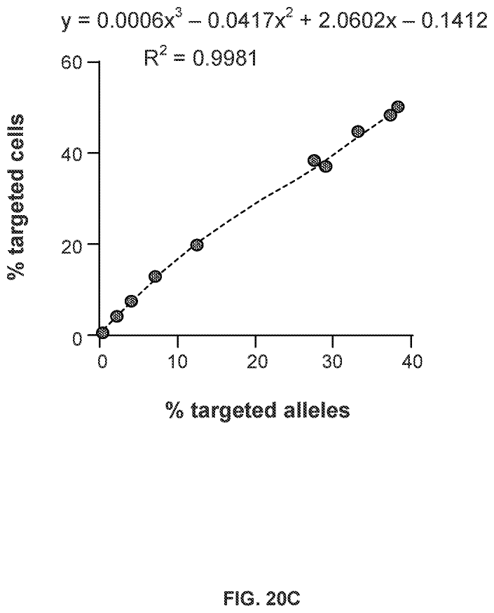

We generated data by dual-color targeting vectors to gain insight into mono- and bi-allelic editing frequencies when targeting HBA1. FIG. 20A shows representative FACS plots of CD34⁺ HSPCs simultaneously targeted by HBA1-WGR-GFP AAV6 and HBA1-WGR-mPlum AAV6. The percentages of populations targeted with GFP only, mPlum only, and both colors were determined (FIG. 20B). The percent edited cells was also plotted against the percent edited alleles for data shown in FIG. 20B (FIG. 20C).

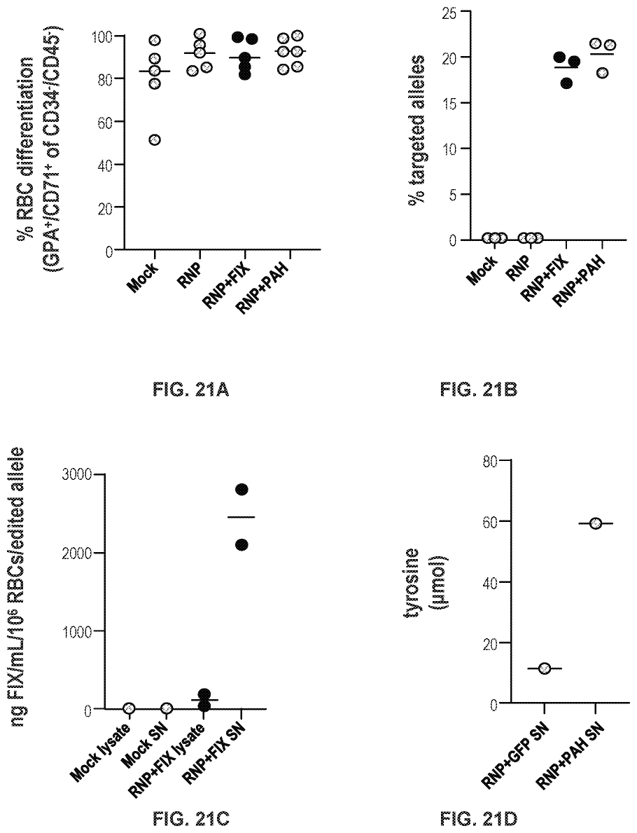

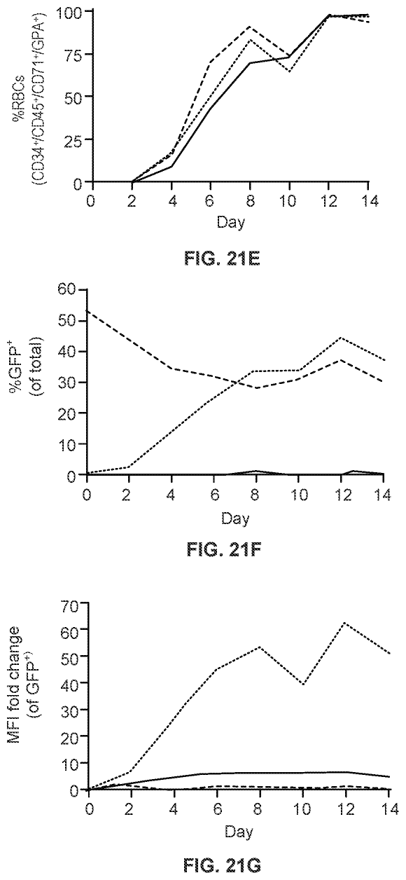

We also obtained updated data for custom transgene integration (using, e.g., PAH or FXI as a transgene) at HBA1 for red blood cell delivery. The percentage of CD34⁻/CD45⁻ HSPCs that acquire RBC surface markers, GPA and CD71, was determined by flow cytometry (FIG. 21A). We also determined the targeted allele frequency at HBA1 in primary HSPCs as determined by ddPCR (FIG. 21B). FIG. 21C shows the FIX production in cell lysate and supernatant following targeting and red blood cell differentiation in primary HSPCs as determined by FIX ELISA, and FIG. 21D shows the production of tyrosine as a proxy for PAH activity in supernatant of 293T cells that were electroporated with transgene-expressing plasmids. The percentage RBCs of primary HSPCs targeted at HBA1 with constitutive GFP and promoterless YFP integration vectors during the course of RBC differentiation was determined by flow cytometry (FIG. 21E), and the percentage GFP of the targeted HSPCs shown in FIG. 21E was also determined. FIG. 21G shows the MFI fold change over d0 measurement of GFP⁺ population shown in FIG. 21F.

### Example 3. Exemplary DNA Donors for Rescuing Disease-Specific Therapeutic Proteins

This example provides several non-limiting examples of donor templates that can be used to knock-in genes at the HBA1 or HBA2 locus.

Mucopolysaccharidosis type 1: to knock in IDUA cDNA to overexpress IDUA enzyme.

Sequence elements:
Left homology arm: 1-500 bp
PGK promoter: 501-1001 bp
IDUA cDNA: 1002-2960 bp
T2A-tNGFR: 2961-3848 bp
BgH Poly A: 3849-4099 bp
Right homology arm: 4100-4599 bp
Sequence:
(SEQ ID NO: 38)
TTTCATGAATTCCCCCAACAGAGCCAAGCTCTCCATCTAGTGGACAGGGA
AGCTAGCAGCAAACCTTCCCTTCACTACAAAACTTCATTGCTTGGCCAAA
AAGAGAGTTAATTCAATGTAGACATCTATGTAGGCAATTAAAAACCTATT
GATGTATAAAACAGTTTGCATTCATGGAGGGCAACTAAATACATTCTAGG
ACTTTATAAAAGATCACTTTTTATTTATGCACAGGGTGGAACAAGATGGA
TTATCAAGTGTCAAGTCCAATCTATGACATCAATTATTATACATCGGAGC
CCTGCCAAAAAATCAATGTGAAGCAAATCGCAGCCCGCCTCCTGCCTCCG
CTCTACTCACTGGTGTTCATCTTTGGTTTTGTGGGCAACATGCTGGTCAT
CCTCATCCTGATAAACTGCAAAAGGCTGAAGAGCATGACTGACATCTACC
TGCTCAACCTGGCCATCTCTGACCTGTTTTTCCTTCTTACTGTCCCCTTC
Tctagataccgggtaggggaggcgcttttcccaaggcagtctggagcatg
cgctttagcagccccgctgggcacttggcgctacacaagtggcctctggc
ctcgcacacattccacatccaccggtaggcgccaaccggctccgttcttt
ggtggccccttcgcgccaccttctactcctcccctagtcaggaagttccc
ccccgccccgcagctcgcgtcgtgcaggacgtgacaaatggaagtagcac
gtctcactagtctcgtgcagatggacagcaccgctgagcaatggaagcgg
gtaggcctttggggcagcggccaatagcagctttgctccttcgctttctg
ggctcagaggctgggaaggggtgggtccgggggcgggctcaggggcgggc
tcaggggcggggcgggcgcccgaaCCCTGCGCCCCCGCGCCGCGCTGCTG
GCGCTCCTGGCCTCGCTCCTGGCCGCGCCCCCGGTGGCCCCGGCCGAGGC
CCCGCACCTGGTGCATGTGGACGCGGCCCGCGCGCTGTGGCCCCTGCGGC
GCTTCTGGAGGAGCACAGGCTTCTGCCCCCCGCTGCCACACAGCCAGGCT
GACCAGTACGTCCTCAGCTGGGACCAGCAGCTCAACCTCGCCTATGTGGG
CGCCGTCCCTCACCGCGGCATCAAGCAGGTCCGGACCCACTGGCTGCTGG
AGCTTGTCACCACCAGGGGGTCCACTGGACGGGGCCTGAGCTACAACTTC
ACCCACCTGGACGGGTACCTGGACCTTCTCAGGGAGAACCAGCTCCTCCC
AGGGTTTGAGCTGATGGGCAGCGCCTCGGGCCACTTCACTGACTTTGAGG
ACAAGCAGCAGGTGTTTGAGTGGAAGGACTTGGTCTCCAGCCTGGCCAGG
AGATACATCGGTAGGTACGGACTGGCGCATGTTTCCAAGTGGAACTTCGA
GACGTGGAATGAGCCAGACCACCACGACTTTGACAACGTCTCCATGACCA
TGCAAGGCTTCCTGAACTACTACGATGCCTGCTCGGAGGGTCTGCGCGCC
GCCAGCCCCGCCCTGCGGCTGGGAGGCCCCGGCGACTCCTTCCACACCCC
ACCGCGATCCCCGCTGAGCTGGGGCCTCCTGCGCCACTGCCACGACGGTA
CCAACTTCTTCACTGGGGAGGCGGGCGTGCGGCTGGACTACATCTCCCTC
CACAGGAAGGGTGCGCGCAGCTCCATCTCCATCCTGGAGCAGGAGAAGGT
CGTCGCGCAGCAGATCCGGCAGCTCTTCCCCAAGTTCGCGGACACCCCCA
TTTACAACGACGAGGCGGACCCGCTGGTGGGCTGGTCCCTGCCACAGCCG
TGGAGGGCGGACGTGACCTACGCGGCCATGGTGGTGAAGGTCATCGCGCA
GCATCAGAACCTGCTACTGGCCAACACCACCTCCGCCTTCCCCTACGCGC
TCCTGAGCAACGACAATGCCTTCCTGAGCTACCACCCGCACCCCTTCGCG
CAGCGCACGCTCACCGCGCGCTTCCAGGTCAACAACACCCGCCCGCCGCA
CGTGCAGCTGTTGCGCAAGCCGGTGCTCACGGCCATGGGGCTGCTGGCGC
TGCTGGATGAGGAGCAGCTCTGGGCCGAAGTGTCGCAGGCCGGGACCGTC
CTGGACAGCAACCACACGGTGGGCGTCCTGGCCAGCGCCCACCGCCCCCA
GGGCCCGGCCGACGCCTGGCGCGCCGCGGTGCTGATCTACGCGAGCGACG
ACACCCGCGCCCACCCCAACCGCAGCGTCGCGGTGACCCTGCGGCTGCGC
GGGGTGCCCCCCGGCCCGGGCCTGGTCTACGTCACGCGCTACCTGGACAA
CGGGCTCTGCAGCCCCGACGGCGAGTGGCGGCGCCTGGGCCGGCCCGTCT
TCCCCACGGCAGAGCAGTTCCGGCGCATGCGCGCGGCTGAGGACCCGGTG
GCCGCGGCGCCCCGCCCCTTACCCGCCGGCGGCCGCCTGACCCTCAGACC
TGCACTTAGATTGCCTTCCCTTTTGTTGGTCCACGTTTGCGCTAGGCCCG
AGAAACCGCCAGGACAAGTAACACGGCTTCGGGCGCTGCCACTTACTCAG
GGGCAGCTGGTGCTGGTTTGGTCAGACGAGCATGTCGGAAGCAAATGCCT
TTGGACCTACGAGATACAATTTTCACAGGATGGTAAGGCTTACACTCCGG
TCTCAAGAAAGCCCAGTACCTTTAACCTTTTTGTGTTCAGTCCAGATACT
GGAGCAGTAAGCGGTTCATATAGAGTCAGAGCGCTGGATTACTGGGCCAG
GCCCGGACCTTTCTCAGATCCGGTCCCCTACCTGGAAGTTCCCGTGCCGC
GGGGTCCTCCATCACCAGGCAACCCAGGAAGCGGAGCTACTAACTTCAGC
CTGCTGAAGCAGGCTGGAGACGTGGAGGAGAACCCTGGACCTGGGGCAGG
TGCCACCGGCCGCGCCATGGACGGGCCGCGCCTGCTGCTGTTGCTGCTTC
TGGGGGTGTCCCTTGGAGGTGCCAAGGAGGCATGCCCCACAGGCCTGTAC
ACACACAGCGGTGAGTGCTGCAAAGCCTGCAACCTGGGCGAGGGTGTGGC
CCAGCCTTGTGGAGCCAACCAGACCGTGTGTGAGCCCTGCCTGGACAGCG
TGACGTTCTCCGACGTGGTGAGCGCGACCGAGCCGTGCAAGCCGTGCACC
GAGTGCGTGGGGCTCCAGAGCATGTCGGCGCCaTGCGTGGAGGCCGACGA
CGCCGTGTGCCGCTGCGCCTACGGCTACTACCAGGATGAGACGACTGGGC
GCTGCGAGGCGTGCCGCGTGTGCGAGGCGGGCTCGGGCCTCGTGTTCTCC
TGCCAGGACAAGCAGAACACCGTGTGCGAGGAGTGCCCCGACGGCACGTA
TTCCGACGAGGCCAACCACGTGGACCCGTGCCTGCCCTGCACCGTGTGCG
AGGACACCGAGCGCCAGCTCCGCGAGTGCACACGCTGGGCCGACGCCGAG
TGCGAGGAGATCCCTGGCCGTTGGATTACACGGTCCACACCCCCAGAGGG
CTCGGACAGCACAGCCCCCAGCACCCAGGAGCCTGAGGCACCTCCAGAAC
AAGACCTCATAGCCAGCACGGTGGCGGGTGTGGTGACCACAGTGATGGGC
AGCTCCCAGCCCGTGGTGACCCGAGGCACCACCGACAACCTCATCCCTGT
CTATTGCTCCATCCTGGCTGCTGTGGTTGTGGGTCTTGTGGCCTACATAG
CCTTCAAGAGGTAAtaacTCGAGCCGCTGAtcagcctcgactgtgccttc
tagttgccagccatctgttgtttgcccctcccccgtgccttccttgaccc
tggaaggtgccactcccactgtcctttcctaataaaatgaggaaattgca
tcgcattgtctgagtaggtgtcattctattctggggggtggggtggggca
ggacagcaagggggaggattgggaagacaatagcaggcatgctggggatg
cggtgggctactagttgggctcactatgctgccgcccagtgggactttgg
aaatacaatgtgtcaactcttgacagggctctattttataggcttcttct
ctggaatcttcttcatcatcctcctgacaatcgataggtacctggctgtc
gtccatgctgtgtttgctttaaaagccaggacggtcacctttggggtggt
gacaagtgtgatcacttgggtggtggctgtgtttgcgtctctcccaggaa
tcatctttaccagatctcaaaaagaaggtcttcattacacctgcagctct
cattttccatacagtcagtatcaattctggaagaatttccagacattaaa
gatagtcatcttggggctggtcctgccgctgcttgtcatggtcatctgct
actcgggaatcctaaaaactctgcttcggtgtcgaaatgagaagaagagg
cacagggctgtgaggcttatcttcaccatcatgattgtttattttctctt
ctgggctccctacaa

Wound Healing Factors: to knock in PGDFB to overexpress protein.

Sequence elements:
Left homology arm: 1-538 bp
SFFV promoter: 539-1083 bp
PDGF-b cDNA: 1084-1806 bp
T2A-GFP: 1807-2550 bp
BgHPolyA: 2551-2835 bp
Right homology arm: 2836-3255 bp
Sequence:
(SEQ ID NO: 39)
GTCCTGTAAGTATTTTGCATATTCTGGAGACGCAGGAAGAGATCCATCTA
CATATCCCAAAGCTGAATTATGGTAGACAAAACTCTTCCACTTTTAGTGC
ATCAACTTCTTATTTGTGTAATAAGAAAATTGGGAAAACGATCTTCAATA
TGCTTACCAAGCTGTGATTCCAAATATTACGTAAATACACTTGCAAAGGA
GGATGTTTTTAGTAGCAATTTGTACTGATGGTATGGGGCCAAGAGATATA
TCTTAGAGGGAGGGCTGAGGGTTTGAAGTCCAACTCCTAAGCCAGTGCCA
GAAGAGCCAAGGACAGGTACGGCTGTCATCACTTAGACCTCACCCTGTGG
AGCCACACCCTAGGGTTGGCCAATCTACTCCCAGGAGCAGGGAGGGCAGG
AGCCAGGGCTGGGCATAAAAGTCAGGGCAGAGCCATCTATTGCTTACATT
TGCTTCTGACACAACTGTGTTCACTAGCAACCTCAAACAGACACCATGGT
GCAcCTGACTCCTGAGGAGAAGTCTGCCGTTACTGCCCattaccctgtta
tccctaccgataaaataaaagattttatttagtctccagaaaaagggggg
aatgaaagaccccacctgtaggtttggcaagctagctgcagtaacgccat
tttgcaaggcatggaaaaataccaaaccaagaatagagaagttcagatca
agggcgggtacatgaaaatagctaacgttgggccaaacaggatatctgcg
gtgagcagtttcggccccggcccggggccaagaacagatggtcaccgcag
tttcggccccggcccgaggccaagaacagatggtccccagatatggccca
accctcagcagtttcttaagacccatcagatgtttccaggctcccccaag
gacctgaaatgaccctgcgccttatttgaattaaccaatcagcctgcttc
tcgcttctgttcgcgcgcttctgcttcccgagctctataaaagagctcac
aacccctcactcggcgcgccagtcctccgacagactgagtcgcccggggg
ggtaccgagctcttcgaaggatccatcgccaccATGAATCGCTGCTGGGC
GCTCTTCCTGTCTCTCTGCTGCTACCTGCGTCTGGTCAGCGCCGAGGGGG
ACCCCATTCCCGAGGAGCTTTATGAGATGCTGAGTGACCACTCGATCCGC
TCCTTTGATGATCTCCAACGCCTGCTGCACGGAGACCCCGGAGAGGAAGA
TGGGGCCGAGTTGGACCTGAACATGACCCGCTCCCACTCTGGAGGCGAGC
TGGAGAGCTTGGCTCGTGGAAGAAGGAGCCTGGGTTCCCTGACCATTGCT
GAGCCGGCCATGATCGCCGAGTGCAAGACGCGCACCGAGGTGTTCGAGAT
CTCCCGGCGCCTCATAGACCGCACCAACGCCAACTTCCTGGTGTGGCCGC
CCTGTGTGGAGGTGCAGCGCTGCTCCGGCTGCTGCAACAACCGCAACGTG
CAGTGCCGCCCCACCCAGGTGCAGCTGCGACCTGTCCAGGTGAGAAAGAT
CGAGATTGTGCGGAAGAAGCCAATCTTTAAGAAGGCCACGGTGACGCTGG
AAGACCACCTGGCATGCAAGTGTGAGACAGTGGCAGCTGCACGGCCTGTG
ACCCGAAGCCCGGGGGGTTCCCAGGAGCAGCGAGCCAAAACGCCCCAAAC
TCGGGTGACCATTCGGACGGTGCGAGTCCGCCGGCCCCCCAAGGGCAAGC
ACCGGAAATTCAAGCACACGCATGACAAGACGGCACTGAAGGAGACCCTT
GGAGCCGGCAGCGGCGAGGGCCGCGGCAGCCTGCTGACCTGCGGCGACGT
GGAGGAGAACCCCGGCCCCATGCCCGCCATGAAGATCGAGTGCCGCATCA
CCGGCACCCTGAACGGCGTGGAGTTCGAGCTGGTGGGCGGCGGAGAGGGC
ACCCCCGAGCAGGGCCGCATGACCAACAAGATGAAGAGCACCAAAGGCGC
CCTGACCTTCAGCCCCTACCTGCTGAGCCACGTGATGGGCTACGGCTTCT
ACCACTTCGGCACCTACCCCAGCGGCTACGAGAACCCCTTCCTGCACGCC
ATCAACAACGGCGGCTACACCAACACCCGCATCGAGAAGTACGAGGACGG
CGGCGTGCTGCACGTGAGCTTCAGCTACCGCTACGAGGCCGGCCGCGTGA
TCGGCGACTTCAAGGTGGTGGGCACCGGCTTCCCCGAGGACAGCGTGATC
TTCACCGACAAGATCATCCGCAGCAACGCCACCGTGGAGCACCTGCACCC
CATGGGCGATAACGTGCTGGTGGGCAGCTTCGCCCGCACCTTCAGCCTGC
GCGACGGCGGCTACTACAGCTTCGTGGTGGACAGCCACATGCACTTCAAG
AGCGCCATCCACCCCAGCATCCTGCAGAACGGGGGCCCCATGTTCGCCTT
CCGCCGCGTGGAGGAGCTGCACAGCAACACCGAGCTGGGCATCGTGGAGT
ACCAGCACGCCTTCAAGACCCCCATCGCCTTCGCCAGATCTCGAGTCTAG
ctcgagggcgcgccCGCTGATCAGCCTCGACCTGTGCCTTCTAGTTGCCA
GCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGT
CTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCT
CTATGGCTTCTGAGGCGGAAAGAACGTTTCGCGCCTGTGGGGCAAGGTGA
ACGTGGATGAAGTTGGTGGTGAGGCCCTGGGCAGGTTGGTATCAAGGTTA
CAAGACAGGTTTAAGGAGACCAATAGAAACTGGGCATGTGGAGACAGAGA
AGACTCTTGGGTTTCTGATAGGCACTGACTCTCTCTGCCTATTGGTCTAT
TTTCCCACCCTTAGGCTGCTGGTGGTCTACCCTTGGACCCAGAGGTTCTT
TGAGTCCTTTGGGGATCTGTCCACTCCTGATGCTGTTATGGGCAACCCTA
AGGTGAAGGCTCATGGCAAGAAAGTGCTCGGTGCCTTTAGTGATGGCCTG
GCTCACCTGGACAACCTCAAGGGCACCTTTGCCACACTGAGTGAGCTGCA
CTGTGACAAGCTGCACGTGGATCCTGAGAACTTCAGGGTGAGTCTATGGG
ACGCT

Beta Thalassemia: to knock in HBB gene (including introns) into Exon 1 of HBA1 gene, which replaces the HBA1 gene with the HBB gene.

Sequence elements:
Left homology arm: 1-880 bp
HBB gene: 881-2370 bp
Right homology arm: 2371-3249 bp
Sequence:
(SEQ ID NO: 6)
gctccagccggttccagctattgctttgtttacctgtttaaccagtat
ttacctagcaagtcttccatcagatagcatttggagagctgggggtgt
cacagtgaaccacgacctctaggccagtgggagagtcagtcacacaaa
ctgtgagtccatgacttggggcttagccagcacccaccaccccacgcg
ccaccccacaaccccgggtagaggagtctgaatctggagccgccccca
gcccagccccgtgctttttgcgtcctggtgtttattccttcccggtgc
ctgtcactcaagcacactagtgactatcgccagagggaaagggagctg
caggaagcgaggctggagagcaggaggggctctgcgcagaaattcttt
tgagttcctatgggccagggcgtccgggtgcgcgcattcctctccgcc
ccaggattgggcgaagcctcccggctcgcactcgctcgcccgtgtgtt
ccccgatcccgctggagtcgatgcgcgtccagcgcgtgccaggccggg
gcgggggtgcgggctgactttctccctcgctagggacgctccggcgcc
cgaaaggaaagggtggcgctgcgctccggggtgcacgagccgacagcg
cccgaccccaacgggccggccccgccagcgccgctaccgccctgcccc
cgggcgagcgggatgggcgggagtggagtggcgggtggagggtggaga
cgtcctggcccccgccccgcgtgcacccccaggggaggccgagcccgc
cgcccggccccgcgcaggccccgcccgggactcccctgcggtccaggc
cgcgccccgggctccgcgccagccaatgagcgccgcccggccgggcgt
gcccccgcgccccaagcataaaccctggcgcgctcgcggcccggcact
cttctggtccccacagactcagagagaacccaccATGGTGCATCTGAC
TCCTGAGGAGAAGTCTGCCGTTACTGCCCTGTGGGGCAAGGTGAACGT
GGATGAAGTTGGTGGTGAGGCCCTGGGCAGgttggtatcaaggttaca
agacaggtttaaggagaccaatagaaactgggcatgtggagacagaga
agactcttgggtttctgataggcactgactctctctgcctattggtct
attttcccacccttagGCTGCTGGTGGTCTACCCTTGGACCCAGAGGT
TCTTTGAGTCCTTTGGGGATCTGTCCACTCCTGATGCTGTTATGGGCA
ACCCTAAGGTGAAGGCTCATGGCAAGAAAGTGCTCGGTGCCTTTAGTG
ATGGCCTGGCTCACCTGGACAACCTCAAGGGCACCTTTGCCACACTGA
GTGAGCTGCACTGTGACAAGCTGCACGTGGATCCTGAGAACTTCAGGg
tgagtctatgggacgcttgatgttttctttccccttcttttctatggt
taagttcatgtcataggaaggggataagtaacagggtacagtttagaa
tgggaaacagacgaatgattgcatcagtgtggaagtctcaggatcgtt
ttagtttcttttatttgctgttcataacaattgttttcttttgtttaa
ttcttgctttctttttttttcttctccgcaatttttactattatactt
aatgccttaacattgtgtataacaaaaggaaatatctctgagatacat
taagtaacttaaaaaaaaactttacacagtctgcctagtacattacta
tttggaatatatgtgtgcttatttgcatattcataatctccctacttt
attttcttttatttttaattgatacataatcattatacatatttatgg
gttaaagtgtaatgttttaatatgtgtacacatattgaccaaatcagg
gtaattttgcatttgtaattttaaaaaatgctttcttcttttaatata
cttttttgtttatcttatttctaatactttccctaatctctttctttc
agggcaataatgatacaatgtatcatgcctctttgcaccattctaaag
aataacagtgataatttctgggttaaggcaatagcaatatctctgcat
ataaatatttctgcatataaattgtaactgatgtaagaggtttcatat
tgctaatagcagctacaatccagctaccattctgcttttattttatgg
ttgggataaggctggattattctgagtccaagctaggcccttttgcta
atcatgttcatacctcttatcttcctcccacagCTCCTGGGCAACGTG
CTGGTCTGTGTGCTGGCCCATCACTTTGGCAAAGAATTCACCCCACCA
GTGCAGGCTGCCTATCAGAAAGTGGTGGCTGGTGTGGCTAATGCCCTG
GCCCACAAGTATCACTAAtggccatgcttcttgccccttgggcctccc
cccagcccctcctccccttcctgcacccgtacccccgtggtctttgaa
taaagtctgagtgggcggcagcctgtgtgtgcctgagttttttccctc
agcaaacgtgccaggcatgggcgtggacagcagctgggacacacatgg
ctagaacctctctgcagctggatagggtaggaaaaggcaggggcggga
ggaggggatggaggagggaaagtggagccaccgcgaagtccagctgga
aaaacgctggaccctagagtgctttgaggatgcatttgctctttcccg
agttttattcccagacttttcagattcaatgcaggtttgctgaaataa
tgaatttatccatctttacgtttctgggcactctgtgccaagaactgg
ctggctttctgcctgggacgtcactggtttcccagaggtcctcccaca
tatgggtggtgggtaggtcagagaagtcccactccagcatggctgcat
tgatcccccatcgttcccactagtctccgtaaaacctcccagatacag
gcacagtctagatgaaatcaggggtgcggggtgcaactgcaggcccca
ggcaattcaataggggctctactttcacccccaggtcaccccagaatg
ctcacacaccagacactgacgccctggggctgtcaagatcaggcgttt
gtctctgggcccagctcagggcccagctcagcacccactcagctcccc
tgaggctggggagcctgtcccattgcgactggagaggagagcggggcc
acagaggcctggctagaaggtcccttctccctggtgtgtgttttctct
ctgctgagcaggcttgcagtgcctggggtatca

### REFERENCES

1. Galanello, R. & Origa, R. Beta-thalassemia. Orphanet J Rare Dis 5, 11 (2010).
2. Mentzer, W. C. & Kan, Y. W. Prospects for research in hematologic disorders: sickle cell disease and thalassemia. JAMA 285, 640-642 (2001).
3. Ehlers, K. H., Giardina, P. J., Lesser, M. L., Engle, M. A. & Hilgartner, M. W. Prolonged survival in patients with beta-thalassemia major treated with deferoxamine. J Pediatr 118, 540-545 (1991).
4. Modell, B., Khan, M. & Darlison, M. Survival in beta-thalassaemia major in the UK: data from the UK Thalassaemia Register. Lancet 355, 2051-2052 (2000).
5. Mettananda, S., Gibbons, R. J. & Higgs, D. R. alpha-Globin as a molecular target in the treatment of beta-thalassemia. Blood 125, 3694-3701 (2015).
6. Dye, D. E., Brameld, K. J., Maxwell, S., Goldblatt, J. & O'Leary, P. The impact of single gene and chromosomal disorders on hospital admissions in an adult population. J Community Genet 2, 81-90 (2011).
7. Fleischhauer, K., et al. Graft rejection after unrelated donor hematopoietic stem cell transplantation for thalassemia is associated with nonpermissive HLA-DPB1 disparity in host-versus-graft direction. Blood 107, 2984-2992 (2006).
8. Puthenveetil, G., et al. Successful correction of the human beta-thalassemia major phenotype using a lentiviral vector. Blood 104, 3445-3453 (2004).
9. Negre, O., et al. Gene Therapy of the beta-Hemoglobinopathies by Lentiviral Transfer of the beta(A(T87Q))-Globin Gene. Hum Gene Ther 27, 148-165 (2016).
10. Breda, L., et al. Therapeutic hemoglobin levels after gene transfer in beta-thalassemia mice and in hematopoietic cells of beta-thalassemia and sickle cells disease patients. PLoS One 7, e32345 (2012).
11. Cavazzana-Calvo, M., et al. Transfusion independence and HMGA2 activation after gene therapy of human beta-thalassaemia. Nature 467, 318-322 (2010).
12. Hacein-Bey-Abina, S., et al. LMO2-associated clonal T cell proliferation in two patients after gene therapy for SCID-X1. Science 302, 415-419 (2003).
13. Howe, S. J., et al. Insertional mutagenesis combined with acquired somatic mutations causes leukemogenesis following gene therapy of SCID-X1 patients. J Clin Invest 118, 3143-3150 (2008).
14. Braun, C. J., et al. Gene therapy for Wiskott-Aldrich syndrome—long-term efficacy and genotoxicity. Sci Transl Med 6, 227ra233 (2014).
15. Stein, S., et al. Genomic instability and myelodysplasia with monosomy 7 consequent to EVI1 activation after gene therapy for chronic granulomatous disease. Nat Med 16, 198-204 (2010).
16. Canver, M. C., et al. BCL11A enhancer dissection by Cas9-mediated in situ saturating mutagenesis. Nature 527, 192-197 (2015).
17. Alter, B. P. Fetal erythropoiesis in stress hematopoiesis. Exp Hematol 7 Suppl 5, 200-209 (1979).
18. Stamatoyannopoulos, G., Veith, R., Galanello, R. & Papayannopoulou, T. Hb F production in stressed erythropoiesis: observations and kinetic models. Ann N Y Acad Sci 445, 188-197 (1985).
19. Bak, R. O., Dever, D. P. & Porteus, M. H. CRISPR/Cas9 genome editing in human hematopoietic stem cells. Nat Protoc 13, 358-376 (2018).
20. Martin, R. M., et al. Highly Efficient and Marker-free Genome Editing of Human Pluripotent Stem Cells by CRISPR-Cas9 RNP and AAV6 Donor-Mediated Homologous Recombination. Cell Stem Cell 24, 821-828 e825 (2019).
21. Pavel-Dinu, M., et al. Gene correction for SCID-X1 in long-term hematopoietic stem cells. Nat Commun 10, 1634 (2019).
22. Gomez-Ospina, N., et al. Human genome-edited hematopoietic stem cells phenotypically correct Mucopolysaccharidosis type I. Nat Commun 10, 4045 (2019).
23. Dever, D. P., et al. CRISPR/Cas9 beta-globin gene targeting in human haematopoietic stem cells. Nature 539, 384-389 (2016).
24. Pal, C., Papp, B. & Lercher, M. J. An integrated view of protein evolution. Nat Rev

Genet 7, 337-348 (2006).
25. Rubin, J. E., Pasceri, P., Wu, X., Leboulch, P. & Ellis, J. Locus control region activity by 5′HS3 requires a functional interaction with beta-globin gene regulatory elements: expression of novel beta/gamma-globin hybrid transgenes. Blood 95, 3242-3249 (2000).
26. Aznarez, I., et al. Mechanism of Nonsense-Mediated mRNA Decay Stimulation by Splicing Factor SRSF1. Cell Rep 23, 2186-2198 (2018).
27. Thein, S. L. The molecular basis of beta-thalassemia. Cold Spring Harb Perspect Med 3, a011700 (2013).
28. Weatherall, D. 2003 William Allan Award address. The Thalassemias: the role of molecular genetics in an evolving global health problem. Am J Hum Genet 74, 385-392 (2004).
29. Hendel, A., et al. Chemically modified guide RNAs enhance CRISPR-Cas genome editing in human primary cells. Nat Biotechnol 33, 985-989 (2015).
30. Brinkman, E. K., Chen, T., Amendola, M. & van Steensel, B. Easy quantitative assessment of genome editing by sequence trace decomposition. Nucleic Acids Res 42, e168 (2014).
31. Vakulskas, C. A., et al. A high-fidelity Cas9 mutant delivered as a ribonucleoprotein complex enables efficient gene editing in human hematopoietic stem and progenitor cells. Nat Med 24, 1216-1224 (2018).
32. Cradick, T. J., Qiu, P., Lee, C. M., Fine, E. J. & Bao, G. COSMID: A Web-based Tool for Identifying and Validating CRISPR/Cas Off-target Sites. Mol Ther Nucleic Acids 3, e214 (2014).
33. Charlesworth, C. T., et al. Priming Human Repopulating Hematopoietic Stem and Progenitor Cells for Cas9/sgRNA Gene Targeting. Mol Ther Nucleic Acids 12, 89-104 (2018).
34. Hubbard, N., et al. Targeted gene editing restores regulated CD40L function in X-linked hyper-IgM syndrome. Blood 127, 2513-2522 (2016).
35. Dulmovits, B. M., et al. Pomalidomide reverses gamma-globin silencing through the transcriptional reprogramming of adult hematopoietic progenitors. Blood 127, 1481-1492 (2016).
36. Hu, J., et al. Isolation and functional characterization of human erythroblasts at distinct stages: implications for understanding of normal and disordered erythropoiesis in vivo. Blood 121, 3246-3253 (2013).
37. Verrier, J. D., et al. Bicistronic lentiviruses containing a viral 2A cleavage sequence reliably co-express two proteins and restore vision to an animal model of LCA1. PLoS One 6, e20553 (2011).
38. Marktel, S., et al. Intrabone hematopoietic stem cell gene therapy for adult and pediatric patients affected by transfusion-dependent ss-thalassemia. Nat Med 25, 234-241 (2019).
39. Schiroli, G., et al. Precise Gene Editing Preserves Hematopoietic Stem Cell Function following Transient p53-Mediated DNA Damage Response. Cell Stem Cell 24, 551-565 e558 (2019).
40. Pattabhi, S., et al. In vivo Outcome of Homology-Directed Repair at the HBB Gene in HSC Using Alternative Donor Template Delivery Methods. Mol Ther Nucleic Acids 17, 277-288 (2019).
41. Bak, R. O., et al. Multiplexed genetic engineering of human hematopoietic stem and progenitor cells using CRISPR/Cas9 and AAV6. Elife 6(2017).
42. Andreani, M., et al. Persistence of mixed chimerism in patients transplanted for the treatment of thalassemia. Blood 87, 3494-3499 (1996).
43. Khan, I. F., Hirata, R. K. & Russell, D. W. AAV-mediated gene targeting methods for human cells. Nat Protoc 6, 482-501 (2011).
44. Aurnhammer, C., et al. Universal real-time PCR for the detection and quantification of adeno-associated virus serotype 2-derived inverted terminal repeat sequences. Hum Gene Ther Methods 23, 18-28 (2012).
45. Cromer, M. K., et al. Global Transcriptional Response to CRISPR/Cas9-AAV6-Based Genome Editing in CD34(+) Hematopoietic Stem and Progenitor Cells. Mol Ther 26, 2431-2442 (2018).
46. Bak, R. O. & Porteus, M. H. CRISPR-Mediated Integration of Large Gene Cassettes Using AAV Donor Vectors. Cell Rep 20, 750-756 (2017).
47. Magoc, T. & Salzberg, S. L. FLASH: fast length adjustment of short reads to improve genome assemblies. Bioinformatics 27, 2957-2963 (2011).
48. Li, H. Minimap2: pairwise alignment for nucleotide sequences. Bioinformatics 34, 3094-3100 (2018).
49. Quinlan, A. R. & Hall, I. M. BEDTools: a flexible suite of utilities for comparing genomic features. Bioinformatics 26, 841-842 (2010).
50. PCT Publication No. WO2020208223.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

INFORMAL (PARTIAL) SEQUENCE LISTING

SEQ ID NO: 1
sg1 target sequence:
5′CTACCGAGGCTCCAGCTTAA-3′
SEQ ID NO: 2
sg2 target sequence:
5′-GGCAGGAGGAACGGCTACCG-3′;
SEQ ID NO: 3
sg3 target sequence:
5′-GGGGAGGAGGGCCCGTTGGG-3′
SEQ ID NO: 4
sg4 target sequence:
5′-CCACCGAGGCTCCAGCTTAA-3′;
SEQ ID NO: 5
sg5 target sequence:
5′-GGCAAGAAGCATGGCCACCG-3′
SEQ ID NO: 6
Donor template to knock in HBB gene (including introns) into Exon l of HBA1 gene,
replacing the HBA1 gene with the HBB gene.
Sequence elements:
Left homology arm: 1-880 bp
HBB gene: 881-2370 bp
Right homology arm: 2371-3249 bp
gctccagccggttccagctattgctttgtttacctgtttaaccagtatttacctagcaagtcttccatcagatagcatttggagagctggggg
tgtcacagtgaaccacgacctctaggccagtgggagagtcagtcacacaaactgtgagtccatgacttggggcttagccagcacccaccaccc
cacgcgccaccccacaaccccgggtagaggagtctgaatctggagccgcccccagcccagccccgtgctttttgcgtcctggtgtttattcct
tcccggtgcctgtcactcaagcacactagtgactatcgccagagggaaagggagctgcaggaagcgaggctggagagcaggaggggctctgcg
cagaaattcttttgagttcctatgggccagggcgtccgggtgcgcgcattcctctccgccccaggattgggcgaagcctcccggctcgcactc
gctcgcccgtgtgttccccgatcccgctggagtcgatgcgcgtccagcgcgtgccaggccggggcgggggtgcgggctgactttctccctcgc
tagggacgctccggcgcccgaaaggaaagggtggcgctgcgctccggggtgcacgagccgacagcgcccgaccccaacgggccggccccgcca
gcgccgctaccgccctgcccccgggcgagcgggatgggcgggagtggagtggcgggtggagggtggagacgtcctggcccccgccccgcgtgc
acccccaggggaggccgagcccgccgcccggccccgcgcaggccccgcccgggactcccctgcggtccaggccgcgccccgggctccgcgcca
gccaatgagcgccgcccggccgggcgtgcccccgcgccccaagcataaaccctggcgcgctcgcggcccggcactcttctggtccccacagac
tcagagagaacccaccATGGTGCATCTGACTCCTGAGGAGAAGTCTGCCGTTACTGCCCTGTGGGGCAAGGTGAACGTGGATGAAGTTGGTGG
TGAGGCCCTGGGCAGgttggtatcaaggttacaagacaggtttaaggagaccaatagaaactgggcatgtggagacagagaagactcttgggt
ttctgataggcactgactctctctgcctattggtctattttcccacccttagGCTGCTGGTGGTCTACCCTTGGACCCAGAGGTTCTTTGAGT
CCTTTGGGGATCTGTCCACTCCTGATGCTGTTATGGGCAACCCTAAGGTGAAGGCTCATGGCAAGAAAGTGCTCGGTGCCTTTAGTGATGGCC
TGGCTCACCTGGACAACCTCAAGGGCACCTTTGCCACACTGAGTGAGCTGCACTGTGACAAGCTGCACGTGGATCCTGAGAACTTCAGGgtga
gtctatgggacgcttgatgttttctttccccttcttttctatggttaagttcatgtcataggaaggggataagtaacagggtacagtttagaa
tgggaaacagacgaatgattgcatcagtgtggaagtctcaggatcgttttagtttcttttatttgctgttcataacaattgttttcttttgtt
taattcttgctttctttttttttcttctccgcaatttttactattatacttaatgccttaacattgtgtataacaaaaggaaatatctctgag
atacattaagtaacttaaaaaaaaactttacacagtctgcctagtacattactatttggaatatatgtgtgcttatttgcatattcataatct
ccctactttattttcttttatttttaattgatacataatcattatacatatttatgggttaaagtgtaatgttttaatatgtgtacacatatt
gaccaaatcagggtaattttgcatttgtaattttaaaaaatgctttcttcttttaatatacttttttgtttatcttatttctaatactttccc
taatctctttctttcagggcaataatgatacaatgtatcatgcctctttgcaccattctaaagaataacagtgataatttctgggttaaggca
atagcaatatctctgcatataaatatttctgcatataaattgtaactgatgtaagaggtttcatattgctaatagcagctacaatccagctac
cattctgcttttattttatggttgggataaggctggattattctgagtccaagctaggcccttttgctaatcatgttcatacctcttatcttc
ctcccacagCTCCTGGGCAACGTGCTGGTCTGTGTGCTGGCCCATCACTTTGGCAAAGAATTCACCCCACCAGTGCAGGCTGCCTATCAGAAA
GTGGTGGCTGGTGTGGCTAATGCCCTGGCCCACAAGTATCACTAAtggccatgcttcttgccccttgggcctccccccagcccctcctcccct
tcctgcacccgtacccccgtggtctttgaataaagtctgagtgggcggcagcctgtgtgtgcctgagttttttccctcagcaaacgtgccagg
catgggcgtggacagcagctgggacacacatggctagaacctctctgcagctggatagggtaggaaaaggcaggggcgggaggaggggatgga
ggagggaaagtggagccaccgcgaagtccagctggaaaaacgctggaccctagagtgctttgaggatgcatttgctctttcccgagttttatt
cccagacttttcagattcaatgcaggtttgctgaaataatgaatttatccatctttacgtttctgggcactctgtgccaagaactggctggct
ttctgcctgggacgtcactggtttcccagaggtcctcccacatatgggtggtgggtaggtcagagaagtcccactccagcatggctgcattga
tcccccatcgttcccactagtctccgtaaaacctcccagatacaggcacagtctagatgaaatcaggggtgcggggtgcaactgcaggcccca
ggcaattcaataggggctctactttcacccccaggtcaccccagaatgctcacacaccagacactgacgccctggggctgtcaagatcaggcg
tttgtctctgggcccagctcagggcccagctcagcacccactcagctcccctgaggctggggagcctgtcccattgcgactggagaggagagc
ggggccacagaggcctggctagaaggtcccttctccctggtgtgtgttttctctctgctgagcaggcttgcagtgcctggggtatca
SEQ ID NO: 7
FIX (Padua variant)
gene length (with introns): 2824bp
Sequence:
ATGCAGAGGGTGAACATGATCATGGCTGAGAGCCCTGGCCTGATCACCATCTGCCTGCTGGGCTACCTGCTGTCTGCTGAATGTACAGGTTTG
TTTCCTTTTTTATAATACATTGAGTATGCTTGCCTTTTAGATATAGAAATATCTGATTCTGTCTTCTTCACTAAATTTTGATTACATGATTTG
ACAGCAATATTGAAGAGTCTAACAGCCAGCACCCAGGTTGGTAAGTACTGGTTCTTTGTTAGCTAGGTTTTCTTCTTCTTCACTTTTAAAACT
AAATAGATGGACAATGCTTATGATGCAATAAGGTTTAATAAACACTGTTCAGTTCAGTATTTGGTCATGTAATTCCTGTTAAAAAACAGTCAT
CTCCTTGGTTTAAAAAAATTAAAAGTGGGAAAACAAAGAAATAGCAGAATATAGTGAAAAAAAATAACCACAGTATTTTTGTTTGGACTTACC
ACTTTGAAATCAAATTGGGAAACAAAAGCACAAACAGTGGCCTTATTTACACAAAAAGTCTGATTTTAAGATATGTGACAATTCAAGGTTTCA
GAAGTATGTAAGGAGGTGTGTCTCTAATTTTTTAAATTATATATCTTCAATTTAAAGTTTTAGTTAAAACATAAAGATTAACCTTTCATTAGC
AAGCTGTTAGTTATCACCAAAGCTTTTCATGGATTAGGAAAAAATCATTTTGTCTCTATCTCAAACATCTTGGAGTTGATATTTGGGGAAACA
CAATACTCAGTTGAGTTCCCTAGGGGAGAAAAGCAAGCTTAAGAATTGACACAAAGAGTAGGAAGTTAGCTATTGCAACATATATCACTTTGT
TTTTTCACAACTACAGTGACTTTATTTATTTCCCAGAGGAAGGCATACAGGGAAGAAATTATCCCATTTGGACAAACAGCATGTTCTCACAGT
AAGCACTTATCACACTTACTTGTCAACTTTCTAGAATCAAATCTAGTAGCTGACAGTACCAGGATCAGGGGTGCCAACCCTAAGCACCCCCAG
AAAGCTGACTGGCCCTGTGGTTCCCACTCCAGACATGATGTCAGCTGTGAAATCCACCTCCCTGGACCATAATTAGGCTTCTGTTCTTCAGGA
GACATTTGTTCAAAGTCATTTGGGCAACCATATTCTGAAAACAGCCCAGCCAGGGTGATGGATCACTTTGCAAAGATCCTCAATGAGCTATTT
TCAAGTGATGACAAAGTGTGAAGTTAAGGGCTCATTTGAGAACTTTCTTTTTCATCCAAAGTAAATTCAAATATGATTAGAAATCTGACCTTT
TATTACTGGAATTCTCTTGACTAAAAGTAAAATTGAATTTTAATTCCTAAATCTCCATGTGTATACAGTACTGTGGGAACATCACAGATTTTG
GCTCCATGCCCTAAAGAGAAATTGGCTTTCAGATTATTTGGATTAAAAACAAAGACTTTCTTAAGAGATGTAAAATTTTCATGATGTTTTCTT
TTTTGCTAAAACTAAAGAATTATTCTTTTACATTTCAGTTTTTCTTGATCATGAAAATGCCAACAAAATTCTGAATAGACCAAAGAGGTATAA
CTCTGGCAAGCTTGAAGAGTTTGTACAGGGGAATCTGGAGAGAGAGTGTATGGAAGAGAAGTGCAGCTTTGAGGAAGCCAGAGAAGTGTTTGA
AAATACAGAGAGAACAACTGAATTTTGGAAGCAGTATGTGGATGGTGATCAATGTGAGAGCAATCCCTGCTTGAATGGGGGGAGCTGTAAAGA
TGATATCAACAGCTATGAATGTTGGTGTCCCTTTGGATTTGAGGGGAAAAACTGTGAGCTTGATGTGACCTGTAATATCAAGAATGGCAGGTG
TGAGCAATTTTGCAAGAATTCTGCTGATAACAAAGTGGTCTGTAGCTGCACTGAGGGATATAGGCTGGCTGAAAACCAGAAGAGCTGTGAACC
TGCAGTGCCTTTTCCCTGTGGGAGAGTGTCTGTGAGCCAAACCAGCAAGCTGACTAGGGCTGAAGCAGTCTTTCCTGATGTAGATTATGTGAA
TAGCACTGAGGCTGAGACAATCCTTGACAATATCACTCAGAGCACACAGAGCTTCAATGACTTCACCAGGGTGGTAGGAGGGGAGGATGCCAA
GCCTGGGCAGTTCCCCTGGCAGGTAGTGCTCAATGGAAAAGTGGATGCCTTTTGTGGAGGTTCAATTGTAAATGAGAAGTGGATTGTGACTGC
AGCCCACTGTGTGGAAACTGGAGTCAAGATTACTGTGGTGGCTGGAGAGCACAATATTGAGGAAACTGAGCACACTGAGCAGAAGAGGAATGT
GATCAGGATTATCCCCCACCACAACTACAATGCTGCTATCAACAAGTACAACCATGACATTGCCCTCCTGGAACTGGATGAACCCCTGGTCTT
GAACAGCTATGTGACACCCATCTGTATTGCTGATAAAGAGTACACCAACATCTTCTTGAAATTTGGGTCTGGATATGTGTCTGGCTGGGGCAG
GGTGTTCCATAAAGGCAGGTCTGCCCTGGTATTGCAGTATTTGAGGGTGCCTCTGGTGGATAGAGCAACCTGCTTGCTGAGCACCAAGTTTAC
AATCTACAACAATATGTTCTGTGCAGGGTTCCATGAAGGTGGTAGAGACAGCTGCCAGGGAGATTCTGGGGGTCCCCATGTGACTGAGGTGGA
GGGAACCAGCTTCCTGACTGGGATTATCAGCTGGGGTGAGGAGTGTGCTATGAAGGGAAAGTATGGGATCTACACAAAAGTATCCAGATATGT
GAACTGGATTAAGGAGAAAACCAAGCTGACTTGA
SEQ ID NO: 8
LDLR
cDNA length (no introns): 2460 bp
Sequence:
ATGGGGCCCTGGGGCTGGAAATTGCGCTGGACCGTCGCCTTGCTCCTCGCCGCGGCGGGGACTGCAGTGGGCGACAGATGCGAAAGAAACGAG
TTCCAGTGCCAAGACGGGAAATGCATCTCCTACAAGTGGGTCTGCGATGGCAGCGCTGAGTGCCAGGATGGCTCTGATGAGTCCCAGGAGACG
TGCTCCCCCAAGACGTGCTCCCAGGACGAGTTTCGCTGCCACGATGGGAAGTGCATCTCTCGGCAGTTCGTCTGTGACTCAGACCGGGACTGC
TTGGACGGCTCAGACGAGGCCTCCTGCCCGGTGCTCACCTGTGGTCCCGCCAGCTTCCAGTGCAACAGCTCCACCTGCATCCCCCAGCTGTGG
GCCTGCGACAACGACCCCGACTGCGAAGATGGCTCGGATGAGTGGCCGCAGCGCTGTAGGGGTCTTTACGTGTTCCAAGGGGACAGTAGCCCC
TGCTCGGCCTTCGAGTTCCACTGCCTAAGTGGCGAGTGCATCCACTCCAGCTGGCGCTGTGATGGTGGCCCCGACTGCAAGGACAAATCTGAC
GAGGAAAACTGCGCTGTGGCCACCTGTCGCCCTGACGAATTCCAGTGCTCTGATGGAAACTGCATCCATGGCAGCCGGCAGTGTGACCGGGAA
TATGACTGCAAGGACATGAGCGATGAAGTTGGCTGCGTTAATGTGACACTCTGCGAGGGACCCAACAAGTTCAAGTGTCACAGCGGCGAATGC
ATCACCCTGGACAAAGTCTGCAACATGGCTAGAGACTGCCGGGACTGGTCAGATGAACCCATCAAAGAGTGCGGGACCAACGAATGCTTGGAC
AACAACGGCGGCTGTTCCCACGTCTGCAATGACCTTAAGATCGGCTACGAGTGCCTGTGCCCCGACGGCTTCCAGCTGGTGGCCCAGCGAAGA
TGCGAAGATATCGATGAGTGTCAGGATCCCGACACCTGCAGCCAGCTCTGCGTGAACCTGGAGGGTGGCTACAAGTGCCAGTGTGAGGAAGGC
TTCCAGCTGGACCCCCACACGAAGGCCTGCAAGGCTGTGGGCTCCATCGCCTACCTCTTCTTCACCAACCGGCACGAGGTCAGGAAGATGACG
CTGGACCGGAGCGAGTACACCAGCCTCATCCCCAACCTGAGGAACGTGGTCGCTCTGGACACGGAGGTGGCCAGCAATAGAATCTACTGGTCT
GACCTGTCCCAGAGAATGATCTGCAGCACCCAGCTTGACAGAGCCCACGGCGTCTCTTCCTATGACACCGTCATCAGCAGAGACATCCAGGCC
CCCGACGGGCTGGCTGTGGACTGGATCCACAGCAACATCTACTGGACCGACTCTGTCCTGGGCACTGTCTCTGTTGCGGATACCAAGGGCGTG
AAGAGGAAAACGTTATTCAGGGAGAACGGCTCCAAGCCAAGGGCCATCGTGGTGGATCCTGTTCATGGCTTCATGTACTGGACTGACTGGGGA
ACTCCCGCCAAGATCAAGAAAGGGGGCCTGAATGGTGTGGACATCTACTCGCTGGTGACTGAAAACATTCAGTGGCCCAATGGCATCACCCTA
GATCTCCTCAGTGGCCGCCTCTACTGGGTTGACTCCAAACTTCACTCCATCTCAAGCATCGATGTCAACGGGGGCAACCGGAAGACCATCTTG
GAGGATGAAAAGAGGCTGGCCCACCCCTTCTCCTTGGCCGTCTTTGAGGACAAAGTATTTTGGACAGATATCATCAACGAAGCCATTTTCAGT
GCCAACCGCCTCACAGGTTCCGATGTCAACTTGTTGGCTGAAAACCTACTGTCCCCAGAGGATATGGTTCTCTTCCACAACCTCACCCAGCCA
AGAGGAGTGAACTGGTGTGAGAGGACCACCCTGAGCAATGGCGGCTGCCAGTATCTGTGCCTCCCTGCCCCGCAGATCAACCCCCACTCGCCC
AAGTTTACCTGCGCCTGCCCGGACGGCATGCTGCTGGCCAGGGACATGAGGAGCTGCCTCACAGAGGCTGAGGCTGCAGTGGCCACCCAGGAG
ACATCCACCGTCAGGCTAAAGGTCAGCTCCACAGCCGTAAGGACACAGCACACAACCACCCGACCTGTTCCCGACACCTCCCGGCTGCCTGGG
GCCACCCCTGGGCTCACCACGGTGGAGATAGTGACAATGTCTCACCAAGCTCTGGGCGACGTTGCTGGCAGAGGAAATGAGAAGAAGCCCAGT
AGCGTGAGGGCTCTGTCCATTGTCCTCCCCATCGTGCTCCTCGTCTTCCTTTGCCTGGGGGTCTTCCTTCTATGGAAGAACTGGCGGCTTAAG
AACATCAACAGCATCAACTTTGACAACCCCGTCTATCAGAAGACCACAGAGGATGAGGTCCACATTTGCCACAACCAGGACGGCTACAGCTAC
CCCTCGAGACAGATGGTCAGTCTGGAGGATGACGTGGCGTGA

1. A method of genetically modifying a hematopoietic stem and progenitor cell (HSPC) from a subject, the method comprising:
introducing into the HSPC a guide RNA comprising a sequence that hybridizes to a HBA1 gene sequence or a HBA2 gene sequence, an RNA-guided nuclease, and a donor template comprising a transgene encoding a protein, wherein
the RNA-guided nuclease cleaves the HBA1 gene sequence or the HBA2 gene sequence, but not both, in the cell; wherein the transgene is integrated into the cleaved HBA1 gene sequence or HBA2 gene sequence; thereby generating a genetically modified HSPC, wherein the integrated transgene results in expression of the protein in the genetically modified HSPC.

2. (canceled)

3. (canceled)

4. The method of claim 1, wherein the method further comprises isolating the HSPC from the subject prior to introducing the guide RNA, the RNA-guided nuclease, and the donor template.

5. The method of claim 1, wherein the HBA1 gene sequence or the HBA2 gene sequence comprises a 3′ UTR region.

6. The method of claim 1, wherein the RNA-guided nuclease cleaves the HBA1 gene sequence but not the HBA2 gene sequence.

7. The method of claim 6, wherein the HBA1 gene sequence comprises a sequence of SEQ ID NO:5.

8. The method of claim 6, wherein the transgene is integrated into the HBA1 gene sequence.

9. The method of claim 1, wherein the RNA-guided nuclease cleaves the HBA2 gene sequence but not the HBA1 gene sequence.

10. The method of claim 9, wherein the HBA2 gene sequence comprises a sequence of SEQ ID NO:2.

11. The method of claim 9, wherein the transgene is integrated into the HBA2 gene sequence.

12. The method of claim 1, wherein the HSPC comprises a HBB gene that comprises a mutation as compared to a wild type HBB gene.

13. The method of claim 12, wherein the mutation is causative of a disease.

14. The method of claim 13, wherein the disease is beta-thalassemia.

15. The method of claim 1, wherein the transgene is selected from the group consisting of HBB, PDGFB, IDUA, FIX (Padua Variant), LDLR, and PAH.

16. The method of claim 1, wherein the transgene is HBB.

17. The method of claim 16, wherein the HBB is expressed in the HSPC and increases a level of adult hemoglobin tetramers in the HSPC as compared to prior to introduction of the guide RNA, the RNA-guided nuclease, and the donor template .

18. The method of claim 16, wherein the transgene is HBB, wherein the guide RNA hybridizes to a sequence of SEQ ID NO:5, and wherein the HBB is integrated at the site of the HBA1 gene sequence.

19. The method of claim 15, wherein the subject has β-thalassemia, and wherein the genetically modified HSPC expressing the HBB transgene is reintroduced into the subject.

20. The method of claim 1, wherein the expression of the integrated transgene is driven by an endogenous HBA1 or HBA2 promoter.

21. The method of claim 1, wherein the integrated transgene replaces the HBA1 or HBA2 coding sequence in a genome of the HSPC.

22. The method of claim 1, wherein the integrated transgene replaces the HBA1 or HBA2 open reading frame (ORF) in a genome of the HSPC.

23. The method of claim 1, wherein the protein is a secreted protein.

24. The method of claim 1, wherein the protein is a therapeutic protein.

25. The method of claim 1, wherein the guide RNA comprises one or more 2′-O-methyl-3′-phosphorothioate (MS) modifications.

26. The method of claim 25, wherein the one or more 2′-O-methyl-3′-phosphorothioate (MS) modifications are present at the three terminal nucleotides of the 5′ and 3′ ends of the guide RNA.

27. The method of claim 1, wherein the RNA-guided nuclease is Cas9.

28. The method of claim 1, wherein the guide RNA and the RNA-guided nuclease are introduced into the HSPC as a ribonucleoprotein (RNP) complex by electroporation.

29. The method of claim 1, wherein the donor template is introduced into the HSPC using a recombinant adeno-associated virus (rAAV) vector.

30. The method of claim 29, wherein the rAAV vector is a AAV6 vector.

31. The method of claim 1, wherein the introducing is performed ex vivo.

32. The method of claim 1, further comprising introducing the genetically modified HSPC into the subject.

33. The method of claim 1, further comprising inducing the genetically modified HSPC to differentiate in vitro or ex vivo into a red blood cell (RBC).

34. The method of claim 1, wherein the subject is a human.

35. A guide RNA comprising a sequence that hybridizes to a HBA1 gene sequence or a HBA2 gene sequence, but not both.

36. The guide RNA of claim 35, wherein the guide RNA hybridizes to a 3′ UTR of the HBA1 gene sequence or the HBA2 gene sequence.

37. The guide RNA of claim 35, wherein the guide RNA hybridizes to the HBA1 gene sequence.

38. The guide RNA of claim 37, wherein the HBA1 gene sequence comprises the sequence of SEQ ID NO: 5.

39. The guide RNA of claim 35, wherein the guide RNA hybridizes to the HBA2 gene sequence.

40. The guide RNA of claim 39, wherein the HBA2 gene sequence comprises the sequence of SEQ ID NO: 2.

41. The guide RNA of claim 35, wherein the guide RNA comprises one or more 2′-O-methyl-3′-phosphorothioate (MS) modifications.

42. The guide RNA of claim 41, wherein the one or more 2′-O-methyl-3′-phosphorothioate (MS) modifications are present at the three terminal nucleotides of the 5′ and 3′ ends of the guide RNA.

43. An HSPC comprising the guide RNA of claim 35.

44-93. (canceled)
